SEQUENCE LISTING (173 sequences)

SEQ ID NO: 1 - protein, 1154 aa, synthetic construct
MGSSHHHHHHHMKRPAATKKAGQAKKKKGGGGSGGGGSGGGGSGSMPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIRGGSGGSGGSGPPKKKRKV

SEQ ID NO: 2 - protein, 1109 aa, synthetic construct
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIRGGSGGSGGSGPPKKKRKV

SEQ ID NO: 3 - protein, 1136 aa, synthetic construct
MGSSHHHHHHHMKRPAATKKAGQAKKKKGGGGSGGGGSGGGGSGSMPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIR

SEQ ID NO: 4 - protein, 1103 aa, synthetic construct
MTIRSMKLKLKIYSGRSAPQLRQGLWRLHRLLNEGTAYYMDWLVHMRQEALPGKSKEEIRAELERRVRQQQEKNGVQNDQVPMDEVLSALRQLYELLVPSAVNNSGDAQTLSRKFLSPLVDPNSEGGKGTSNAGAKPGWRKKQEAGDPSWEKDYERWLKRKQADPTAEILGKLETAGLKPLFPLYTNEVKDIRWMPLTSKQYVRNWDRDMFQQAIEHLLSWETWNRKVNEERAKLKETVRRFEEQHLANGKDWLSPLQAYEANREQALRDMAISPSDRFRITRRQIKGWSELYERWNKLAPTASVEAYMQEVRHVQKKLGGTFGDADLYRFLAKPENVHIWRDHQERLHYYAAYNDLHKRLMSAKEQAAFTLPDPVAHPLWVRFDARDGNLFTYILQADSSKQRSRRYVNFSRFLWPVEDGYFEETENVKVELALSKQFYRQVIVHDNPTGKQKITFQDYSSKEILEGHLGGAKLQLDRNFLRKSGRDFETGDFGPAFLNVVLDLKPKQEVKNGRLQSPLGQALLVKSRPNDIPKVYGYKPDALAAWLEQASGEESLGSESLRQGFRVMSIDLGVRSAAAISVFSVKGEKTREGDKVCYPVGETGLFAVHDRSFLLRLPGESSEKRVNVERDKRKTERMQIRYHIRTLARVLRLANKATPMDRIKAVQDVLNDIESTRFMNDHDHHVYNHALETLRTYAPDHQGIWEEQVIAAHRQLEHHVGVIVGEWRKNWGKDRRGTVGLSMDNIEELDEMRRLLISWSRRARYPREAKPFQVNESNPVHLLRHLQNLKEDRLKQLANLIVMTALGYVYDSKEKKWKAAYPACQLILFEDLQRYRFHLDRSARENSQLMKWAHRSIPKYVWMQGEPYGLQIGDVWAGFTSRYHAKTGAPGIRCKALTEKDFQQGRLLESLVAEGMFTLQEVGTLKPGDIVPAEGGELFVTLADDSGDRIVITHADINAAQNVQKRFWLANSERFRVACRSVQIASQECFIPSSESVAKKMGKGVFVRDFSFHKDMEVYHWNNQVKLTAKNVPTDHSDDLQDLQDYQAILEEARESSSSYKTLFRDPSGFFFPDDVWVPQNIYWREVKKTITALLRKRIMST

SEQ ID NO: 5 - protein, 1091 aa, synthetic construct
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYQKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQRKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIEHEELSDEWEDMQEGYKTLLRDPSGFFWSSDSWIPQKDFWIRVKSRIGKSLREQIR

SEQ ID NO: 6 - protein, 1091 aa, synthetic construct
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIR

SEQ ID NO: 7 - protein, 7 aa, synthetic construct
PKKKRKV

SEQ ID NO: 8 - protein, 16 aa, synthetic construct
KRPAATKKAGQAKKKK

SEQ ID NO: 9 - protein, 9 aa, synthetic construct
PAAKRVKLD

SEQ ID NO: 10 - protein, 25 aa, synthetic construct
MSRRRKANPTKLSENAKKLAKEVEN

SEQ ID NO: 11 - protein, 9 aa, synthetic construct
KLKIKRPVK

SEQ ID NO: 12 - skipped

SEQ ID NO: 13 - protein, 7 aa, synthetic construct
PKKKRMV

SEQ ID NO: 14 - protein, 10 aa, synthetic construct
PKKKRKWEDP

SEQ ID NO: 15 - protein, 13 aa, synthetic construct
CGYGPKKKRKVGG

SEQ ID NO: 16 - protein, 11 aa, synthetic construct
CGYGPKKKRKV

SEQ ID NO: 17 - protein, 34 aa, synthetic construct
CYDDEATADSQHSTPPKKKRKWEDPKDFESELLS

SEQ ID NO: 18 - protein, 11 aa, synthetic construct
CGGPKKKRKWG

SEQ ID NO: 19 - protein, 7 aa, synthetic construct
PKKKIKW

SEQ ID NO: 20 - protein, 18 aa, synthetic construct
KRTADGSEFESPKKKRKV

SEQ ID NO: 21 - protein, 10 aa, synthetic construct
TKKAGQAKKK

SEQ ID NO: 22 - protein, 13 aa, synthetic construct
TKKAGQAKKKKLD

SEQ ID NO: 23 - protein, 10 aa, synthetic construct
CGQAKKKKLD

SEQ ID NO: 24 - protein, 11 aa, synthetic construct
RQRRNELKRSP

SEQ ID NO: 25 - protein, 7 aa, synthetic construct
PKKARED

SEQ ID NO: 26 - protein, 13 aa, synthetic construct
CGYGWSRKRPRPG

SEQ ID NO: 27 - protein, 8 aa, synthetic construct
APTKRKGS

SEQ ID NO: 28 - protein, 11 aa, synthetic construct
APKRKSGVSKC

SEQ ID NO: 29 - protein, 7 aa, synthetic construct
PNKKKRK

SEQ ID NO: 30 - protein, 12 aa, synthetic construct
EEDGPQKKKRRL

SEQ ID NO: 31 - protein, 7 aa, synthetic construct
GKKRSKA

SEQ ID NO: 32 - protein, 5 aa, synthetic construct
KRPRP

SEQ ID NO: 33 - protein, 11 aa, synthetic construct
CGGLSSKRPRP

SEQ ID NO: 34 - protein, 13 aa, synthetic construct
LVRKKRKTEEESP

SEQ ID NO: 35 - protein, 12 aa, synthetic construct
LKDKDAKKSKQE

SEQ ID NO: 36 - protein, 10 aa, synthetic construct
GNKAKRQRST

SEQ ID NO: 37 - protein, 11 aa, synthetic construct
PFLDRLRRDQK

SEQ ID NO: 38 - protein, 9 aa, synthetic construct
SVTKKRKLE

SEQ ID NO: 39 - protein, 9 aa, synthetic construct
SASKRRRLE

SEQ ID NO: 40 - protein, 10 aa, synthetic construct
ACIDKRVKLD

SEQ ID NO: 41 - protein, 12 aa, synthetic construct
SALIKKKKKMAP

SEQ ID NO: 42 - protein, 11 aa, synthetic construct
PPKKRMRRRIE

SEQ ID NO: 43 - protein, 28 aa, synthetic construct
YRKCLQAGMNLEARKTKKKIKGIQQATA

SEQ ID NO: 44 - protein, 13 aa, synthetic construct
CGYGARKTKKKIK

SEQ ID NO: 45 - protein, 17 aa, synthetic construct
RKCLQAGMNLEARKTKK

SEQ ID NO: 46 - protein, 8 aa, synthetic construct
RKFKKFNK

SEQ ID NO: 47 - protein, 13 aa, synthetic construct
CGYGIRKDRRGGR

SEQ ID NO: 48 - protein, 13 aa, synthetic construct
CGYGARKLKKLGN

SEQ ID NO: 49 - protein, 8 aa, synthetic construct
GKRKNKPK

SEQ ID NO: 50 - protein, 8 aa, synthetic construct
PLLKKIKQ

SEQ ID NO: 51 - protein, 8 aa, synthetic construct
PPQKKIKS

SEQ ID NO: 52 - protein, 7 aa, synthetic construct
PQPKKKP

SEQ ID NO: 53 - protein, 8 aa, synthetic construct
PQPKKKPL

SEQ ID NO: 54 - protein, 9 aa, synthetic construct
SKRWAKRKL

SEQ ID NO: 55 - protein, 14 aa, synthetic construct
MTGSKTRKHRGSGA

SEQ ID NO: 56 - protein, 14 aa, synthetic construct
MTGSKHRKHPGSGA

SEQ ID NO: 57 - protein, 6 aa, synthetic construct
RHRKHP

SEQ ID NO: 58 - protein, 6 aa, synthetic construct
KRRKHP

SEQ ID NO: 59 - protein, 6 aa, synthetic construct
KYRKHP

SEQ ID NO: 60 - protein, 6 aa, synthetic construct
KHRRHP

SEQ ID NO: 61 - protein, 6 aa, synthetic construct
KHKKHP

SEQ ID NO: 62 - protein, 6 aa, synthetic construct
RHLKHP

SEQ ID NO: 63 - protein, 6 aa, synthetic construct
KHRKYP

SEQ ID NO: 64 - protein, 6 aa, synthetic construct
KHRQHP

SEQ ID NO: 65 - protein, 13 aa, synthetic construct
LVRKKRKTEEESP

SEQ ID NO: 66 - protein, 12 aa, synthetic construct
LKDKDAKKSKQE

SEQ ID NO: 67 - protein, 9 aa, synthetic construct
ASKSRKRKL

SEQ ID NO: 68 - protein, 16 aa, synthetic construct
GGLCSARLHRHALLAT

SEQ ID NO: 69 - protein, 18 aa, synthetic construct
DTREKKKFLKRRLLRLDE

SEQ ID NO: 70 - protein, 10 aa, synthetic construct
REKKKFLKRR

SEQ ID NO: 71 - protein, 12 aa, synthetic construct
CGYGDRNKKKKE

SEQ ID NO: 72 - protein, 13 aa, synthetic construct
RKRQRALMLRQAR

SEQ ID NO: 73 - protein, 11 aa, synthetic construct
EYLSRKGKLEL

SEQ ID NO: 74 - protein, 8 aa, synthetic construct
KKSKKKRC

SEQ ID NO: 75 - protein, 13 aa, synthetic construct
QPQRYGGGRGRRW

SEQ ID NO: 76 - protein, 35 aa, synthetic construct
NKKKRKLSRGSSQKTKGTSASAKARHKRRNRSSRS

SEQ ID NO: 77 - protein, 19 aa, synthetic construct
RVTIRTWRWRRPPKGKHRK

SEQ ID NO: 78 - protein, 15 aa, synthetic construct
KRKIEEPEPEPKKAK

SEQ ID NO: 79 - protein, 21 aa, synthetic construct
KKYENVVIKRSPRKRGRPRKD

SEQ ID NO: 80 - protein, 22 aa, synthetic construct
GRKRAFHGDDPFGEGPPDKKGD

SEQ ID NO: 81 - protein, 21 aa, synthetic construct
KRPREDDDGEPSERKRARDDR

SEQ ID NO: 82 - protein, 42 aa, synthetic construct
RMRIZFKNKGKDTAELRRRRVEVSVELRKAKKDEQILKRRNV

SEQ ID NO: 83 - protein, 38 aa, synthetic construct
NQSSNFGPMKGGNFGGRSSGPYGGGGQYFAKPRNQGGY

SEQ ID NO: 84 - protein, 20 aa, synthetic construct
KRKGDEVDGVDEVAKKKSKK

SEQ ID NO: 85 - protein, 10 aa, synthetic construct
RKLKKKIKKL

SEQ ID NO: 86 - protein, 7 aa, synthetic construct
PKQKKRK

SEQ ID NO: 87 - protein, 12 aa, synthetic construct
SALIKKKKKMAP

SEQ ID NO: 88 - protein, 8 aa, synthetic construct
VSRKRPRP

SEQ ID NO: 89 - protein, 8 aa, synthetic construct
PPKKARED

SEQ ID NO: 90 - protein, 10 aa, synthetic construct
GPAAKRVKLD

SEQ ID NO: 91 - protein, 8 aa, synthetic construct
KKRRIKQD

SEQ ID NO: 92 - protein, 17 aa, synthetic construct
PKKKRKVEDPKKKRKVD

SEQ ID NO: 93 - protein, 23 aa, synthetic construct
PKKKRKVDPKKKRKVDPKKKRKV

SEQ ID NO: 94 - protein, 8 aa, synthetic construct
KKGKKKGK

SEQ ID NO: 95 - protein, 9 aa, synthetic construct
PKRRRGVVL

SEQ ID NO: 96 - protein, 9 aa, synthetic construct
EQLFKRRNV

SEQ ID NO: 97 - protein, 5 aa, synthetic construct
KRRRR

SEQ ID NO: 98 - protein, 5 aa, synthetic construct
KKRRR

SEQ ID NO: 99 - protein, 10 aa, synthetic construct
EGAPPAKRPR

SEQ ID NO: 100 - protein, 11 aa, synthetic construct
MLRRRRRKRAR

SEQ ID NO: 101 - protein, 5 aa, synthetic construct
RRKRR

SEQ ID NO: 102 - protein, 4 aa, synthetic construct
RKRK

SEQ ID NO: 103 - protein, 12 aa, synthetic construct
FKAVLEDILGEL

SEQ ID NO: 104 - protein, 5 aa, synthetic construct
KNRRL

SEQ ID NO: 105 - protein, 5 aa, synthetic construct
RKRHW

SEQ ID NO: 106 - protein, 6 aa, synthetic construct
RRKKRR

SEQ ID NO: 107 - protein, 6 aa, synthetic construct
RRKRSR

SEQ ID NO: 108 - protein, 6 aa, synthetic construct
KRGRKP

SEQ ID NO: 109 - protein, 6 aa, synthetic construct
KKRKLE

SEQ ID NO: 110 - protein, 7 aa, synthetic construct
PKKKSRK

SEQ ID NO: 111 - protein, 7 aa, synthetic construct
PKRGRGR

SEQ ID NO: 112 - protein, 7 aa, synthetic construct
KEKRKKR

SEQ ID NO: 113 - protein, 7 aa, synthetic construct
KKKKRKR

SEQ ID NO: 114 - protein, 8 aa, synthetic construct
RRGDGRRR

SEQ ID NO: 115 - protein, 8 aa, synthetic construct
LSPSLSPL

SEQ ID NO: 116 - protein, 8 aa, synthetic construct
VNFSEFSK

SEQ ID NO: 117 - protein, 8 aa, synthetic construct
IVINILSE

SEQ ID NO: 118 - protein, 9 aa, synthetic construct
PPAKRKCIF

SEQ ID NO: 119 - protein, 9 aa, synthetic construct
QRPGPYDRP

SEQ ID NO: 120 - protein, 9 aa, synthetic construct
KRKRGRPRK

SEQ ID NO: 121 - protein, 9 aa, synthetic construct
KIKELYRRR

SEQ ID NO: 122 - protein, 10 aa, synthetic construct
MVQLRPRASR

SEQ ID NO: 123 - protein, 10 aa, synthetic construct
KKRREKQRRR

SEQ ID NO: 124 - protein, 10 aa, synthetic construct
EGAPPAKRAR

SEQ ID NO: 125 - protein, 11 aa, synthetic construct
PKKGDKYDKTD

SEQ ID NO: 126 - protein, 11 aa, synthetic construct
KKKKSKDKKRK

SEQ ID NO: 127 - protein, 12 aa, synthetic construct
AHRAKKMSKTHA

SEQ ID NO: 128 - protein, 12 aa, synthetic construct
KKGPSVQKRKKT

SEQ ID NO: 129 - protein, 12 aa, synthetic construct
KGVKRKADTTTP

SEQ ID NO: 130 - protein, 12 aa, synthetic construct
KGVKRRADTTTP

SEQ ID NO: 131 - protein, 13 aa, synthetic construct
KKPKWDDFKKKKK

SEQ ID NO: 132 - protein, 13 aa, synthetic construct
KRRRRRRREKRKR

SEQ ID NO: 133 - protein, 13 aa, synthetic construct
DVRKRVQDLEQKM

SEQ ID NO: 134 - protein, 13 aa, synthetic construct
KKGKDEWFSRGKK

SEQ ID NO: 135 - protein, 14 aa, synthetic construct
KKGKDEWFSRGKKP

SEQ ID NO: 136 - protein, 14 aa, synthetic construct
ASPEYVNLPINGNG

SEQ ID NO: 137 - protein, 14 aa, synthetic construct
YLRPVKKPKIRRKK

SEQ ID NO: 138 - protein, 15 aa, synthetic construct
KRKGKLKNKGSKRKK

SEQ ID NO: 139 - protein, 15 aa, synthetic construct
RRRGKNKVAAQNCRK

SEQ ID NO: 140 - protein, 16 aa, synthetic construct
DKAKRVSRNKSEKKRR

SEQ ID NO: 141 - protein, 16 aa, synthetic construct
EEQLRRRKNSRLNNTG

SEQ ID NO: 142 - protein, 17 aa, synthetic construct
HKKKHPDASVNFSEFSK

SEQ ID NO: 143 - protein, 17 aa, synthetic construct
KKTGKNRKLKSKRVKTR

SEQ ID NO: 144 - protein, 17 aa, synthetic construct
KRSCRRRLAGHNERRRK

SEQ ID NO: 145 - protein, 17 aa, synthetic construct
KRQRRKQSNRESARRSR

SEQ ID NO: 146 - protein, 18 aa, synthetic construct
RGKGGKGLGKGGAKRHRK

SEQ ID NO: 147 - protein, 18 aa, synthetic construct
RRRGFERFGPDNMGRKRK

SEQ ID NO: 148 - protein, 18 aa, synthetic construct
RRHQQGQGDDSSHKKERK

SEQ ID NO: 149 - protein, 18 aa, synthetic construct
KKKTGVIAPKRFVQRLKK

SEQ ID NO: 150 - protein, 19 aa, synthetic construct
KRAMKDDSHGNSTSPKRRK

SEQ ID NO: 151 - protein, 19 aa, synthetic construct
KVNFLDMSLDDIIIYKELE

SEQ ID NO: 152 - protein, 20 aa, synthetic construct
KKYENVVIKRSPRKRGRPRK

SEQ ID NO: 153 - protein, 20 aa, synthetic construct
KRGNSSIGPNDLSKRKQRKK

SEQ ID NO: 154 - protein, 22 aa, synthetic construct
KRASEDTTSGSPPKKSSAGPKR

SEQ ID NO: 155 - protein, 23 aa, synthetic construct
KRIHSVSLSQSQIDPSKKVKRAK

SEQ ID NO: 156 - protein, 24 aa, synthetic construct
EVLKVIRTGKRKKKAWKRMVTKVC

SEQ ID NO: 157 - protein, 29 aa, synthetic construct
IINGRKLKLKKSRRRSSQTSNNSFTSRRS

SEQ ID NO: 158 - protein, 30 aa, synthetic construct
AHFKISGEKRPSTDPGKKAKNPKKKKKKDP

SEQ ID NO: 159 - protein, 15 aa, synthetic construct
GGGGSGGGGSGGGGS

SEQ ID NO: 160 - protein, 18 aa, synthetic construct
GGGEAAAKEAAAKEAAAK

SEQ ID NO: 161 - protein, 46 aa, synthetic construct
AEAAAKEAAAKEAAAKEAAAKALEAEAAAKEAAAKEAAAKEAAAKA

SEQ ID NO: 162 - protein, 16 aa, synthetic construct
GGGGSLVPRGSGGGGS

SEQ ID NO: 163 - protein, 33 aa, synthetic construct
GAAPAAAPAKQEAAAPAPAAKAEAPAAAPAAKA

SEQ ID NO: 164 - protein, 10 aa, synthetic construct
TRHRQPRGWE

SEQ ID NO: 165 - RNA, 97 nt, synthetic construct
  source 1..97: mol_type = other RNA
gtttagctataggctaataagatagttgtgtcaagtgcttcggagacctaacacgtcatccagtcacaacggctatctaaaaatagatggttagcac

SEQ ID NO: 166 - RNA, 87 nt, synthetic construct
  source 1..87: mol_type = other RNA
ttagctataggctaataagatagttgtgtcaagtgcttcggagacctaacacgtcatccagtcacaacggctaaaaatggttagcac

SEQ ID NO: 167 - RNA, 87 nt, synthetic construct
  source 1..87: mol_type = other RNA
ttagctataggctaataagatagttgtgtcaagtgcttcggagacctaacacgtcatccagtcacaacggctaaaaatagccagcac

SEQ ID NO: 168 - RNA, 86 nt, synthetic construct
  source 1..86: mol_type = other RNA
ttagctataggctaataagatagttgtgtcaagtgcttcggagacctaacacgtctccagtcacaacggctaaaaatagccagcac

SEQ ID NO: 169 - RNA, 86 nt, synthetic construct
  source 1..86: mol_type = other RNA
ttagctataggctaataagatagttgtgacaagtgcttcggagacctaacacgtctccagtcacaacggctaaaaatagccagcac

SEQ ID NO: 170 - RNA, 106 nt, synthetic construct
  source 1..106: mol_type = other RNA
taataaataatatgagttacaggctcatgtcattccgtgcgtgtgctgcgaggctcttacaacgtctcaaccacgggatcacgttatttccactaagtgattggac

SEQ ID NO: 171 - RNA, 109 nt, synthetic construct
  source 1..109: mol_type = other RNA
ctggatataataaagatgtttagctataggctaataagatagttgtgtcaagtgcttcggagacctaacacgtcatccagtcacaacggctatctatatttccactaac

SEQ ID NO: 172 - protein, 1143 aa, synthetic construct
MKRPAATKKAGQAKKKKGGGGSGGGGSGGGGSGSMPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIRGGSGGSGGSGPPKKKRKV

SEQ ID NO: 173 - protein, 1125 aa, synthetic construct
MKRPAATKKAGQAKKKKGGGGSGGGGSGGGGSGSMPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIR

(19) United States
(12) Patent Application Publication
(10) Pub. No.: US 2026/0209732 A1
(43) Pub. Date: Jul. 23, 2026

(54) ENGINEERED TYPE V RNA PROGRAMMABLE ENDONUCLEASES AND THEIR USES

(71) Applicant: Bayer Aktiengesellschaft, Leverkusen (DE)

(72) Inventors: Timsi RAO, Cambridge, MA (US); Jon Scott MUNZER, Cambridge, MA (US); Mohamadadnan NURANI, Cambridge, MA (US); Andreas NEERINCX, Cologne (DE); Philipp KNYPHAUSEN, Cologne (DE); André COHNEN, Cologne (DE)

(21) Appl. No.: 19/139,160

(22) PCT Filed: Dec. 12, 2023
(86) PCT No.: PCT/US2023/083646

§ 371 Date: Jun. 13, 2025

Related U.S. Application Data
(60) Provisional application No. 63/432,232, filed on Dec. 13, 2022.

(51) Int. Cl. C12N 9/22; C12N 5/071; C12N 15/10; C12N 15/113; C12N 15/52; C12N 15/62; C12N 15/85

(52) U.S. Cl. CPC C12N 9/222; C12N 5/0602; C12N 15/102; C12N 15/113; C12N 15/52; C12N 15/625; C12N 15/85; C12N 2310/20; C12N 2830/34

(57) ABSTRACT

The present disclosure provides engineered type V nucleases suitable for editing eukaryotic genomic DNA, as well as methods of producing the nucleases, systems comprising the nucleases, as well as methods of using the nucleases and systems to edit eukaryotic genomic DNA.

### 1. CROSS-REFERENCE TO RELATED APPLICATIONS

This application claims the priority benefit of U.S. provisional application No. 63/432,232, filed Dec. 13, 2022, the contents of which are incorporated herein in their entireties by reference thereto.

### 2. SEQUENCE LISTING

The instant application contains a Sequence Listing which has been submitted electronically and is hereby incorporated by reference in its entirety. Said copy, created on Nov. 17, 2023, is named BRT-002WO_SL and is 160,005 bytes in size.

### 3. BACKGROUND

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely diverse group of protein effectors and non-coding elements, as well as loci architectures, some examples of which have been engineered and adapted to produce important biotechnologies.

The components of the system involved in host defense include one or more effector proteins capable of modifying DNA or RNA and an RNA guide element that is responsible for targeting these protein activities to a specific sequence on the phage DNA or RNA. The RNA guide is composed of a CRISPR RNA (crRNA) and may require an additional trans-acting RNA (tracrRNA) to enable targeted nucleic acid manipulation by the effector protein(s). The crRNA consists of a segment termed “direct repeat”, that is responsible for binding of the crRNA to the effector protein, and a segment termed “spacer sequence”, that is complementary to the desired nucleic acid target sequence. CRISPR systems can be reprogrammed to target alternative DNA or RNA targets by modifying the spacer sequence of the crRNA.

CRISPR-Cas systems can be broadly classified into two classes: Class 1 systems are composed of multiple effector proteins that together form a complex around a crRNA, and Class 2 systems consist of a single effector protein that complexes with the crRNA guide to target DNA or RNA substrates. The single-subunit effector composition of the Class 2 systems provides a simpler component set for engineering and application and has thus far been an important source of programmable effectors. Thus, the discovery, engineering, and optimization of novel Class 2 systems may lead to widespread and powerful programmable technologies for genome engineering and beyond.

Editing genomes using the RNA-guided DNA targeting principle of CRISPR (Clustered Regularly Interspaced Short Palindromic Repeats)-Cas (CRISPR associated proteins), has been widely exploited in recent years. Five types of CRISPR-Cas systems (type I, type II and |lb, type Ill, type V, and type VI) have been described. Most uses of CRISPR-Cas for genome editing have been with a type II system. The main advantage provided by the bacterial type II CRISPR-Cas system lies in the minimal requirement for programmable DNA interference: an endonuclease, Cas9, guided by a customizable dual-RNA structure. As initially demonstrated in the original type II system of Streptococcus pyogenes, trans-activating CRISPR RNA (tracrRNA) binds to the invariable repeats of precursor CRISPR RNA (pre-crRNA) forming a dual-RNA that is essential for both crRNA co-maturation by RNase Ill in the presence of Cas9, and invading DNA cleavage by Cas9. As demonstrated in Streptococcus pyogenes, Cas9, guided by the duplex formed between mature activating tracrRNA and targeting crRNA, introduces site-specific double-stranded DNA (dsDNA) breaks in the invading cognate DNA. Cas9 is a multi-domain enzyme that uses an HNH nuclease domain to cleave the target strand (defined as complementary to the spacer sequence of crRNA) and a RuvC-like domain to cleave the non-target strand.

In addition to Type II CRISPR Cas 9 nucleases, a number of different type V CRISPR Cas nucleases have been described, such as Cas12a, Cas12b, Cas12e, Cas12f, Cas13a, Cas13b (Koonin et al., Curr Opin Microbiol. 2017 June; 37: 67-78, and Makarova et al., Nat Rev Microbiol. 2020 February; 18(2):67-83.). Some of these systems require no tracr RNA (Cas 12a, Cas 13a, Cas 13b), whereas Cas 12b nucleases typically require a tracr RNA (Koonin et al., Curr Opin Microbiol. 2017 June; 37: 67-78).

Genome editing in mammalian cells has been limited, in part, by the size of various Cas9 proteins. Cas9 from Staphylococcus pyogenes (SpyCas9), the enzyme most widely used to date, comprises approximately 4.2kb of DNA (WO2013/176722) and a direct combination with cognate single guide RNAs (sgRNA) further increases the size. Adeno-associated viruses are among the vectors used for the delivery of Cas9 enzymes in gene therapy applications. However, AAV cargo size is restricted to about 4.5 kb. Because of the size constraints, delivering a Cas9 with its sgRNA and a potential DNA repair template can be an impediment to using the methods. Smaller Cas9 molecules have been characterized, but most of them suffer from a protospacer adjacent motif (PAM) sequence that is not as well defined as the one used by SpyCas9. For example, Staphylococcus aureus (SauCas9 uses an “NNGRR(T)” sequence, where R=A or G, and Campylobacter jejuni (Cja) Cas9 uses a “NNNACAC”/“NNNRYAC” PAM (where Y=T or G), respectively. The PAM ambiguity increases the potential for undesirable activity of the enzyme at off-target sequences harboring high or perfect sequence identity to the PAM. Specificity of these systems remains a concern, as targeting similar sites by accident (“off-targets”) will increase the likelihood of adverse events.

Existing CRISPR-Cas systems generally have one or more of the following disadvantages:
a) Their size is too large to be carried inside the genome of established therapeutically suitable viral delivery systems like adeno associated viruses (AAVs).
b) Many of them are not substantially active in non-host environments, for example in eukaryotic cells, and in particular, in mammalian cells.
c) Their nuclease can catalyze DNA strand cleavage when mismatches between spacer and protospacer sequences are present, leading to undesired off target effects that would for example make them unsuitable for gene therapeutic uses or other applications requiring high precision.
d) They may trigger an immune response that can limit their use for in vivo applications in mammals.
e) They require complex and/or long PAMs that restrict target selection for the DNA targeting segments.
f) They exhibit poor expression from plasmid or viral vectors.
g) They require additional RNA sequences to be active or additional RNA sequences as part of the guide RNA.

The present disclosure provides novel engineered type V nucleases suitable for use in systems that address one or more of the preceding disadvantages suffered by existing CRISPR-Cas systems.

### 4. SUMMARY

The present invention relates to engineered Type V CRISPR Cas nucleases named B-GEn, comprising nuclease sequences relating to B-GEn.1 (SEQ ID NO:4), B-Gen1.2 (SEQ ID NO: 5) and B-GEn.2 (SEQ ID NO: 6), one or more nuclear localization sequences (NLSs), and optionally one or more linker sequences connecting the nuclease sequence and the NLS sequence(s) or a plurality of NLS sequence(s). Exemplary engineered B-GEn polypeptides are described in Section 6.2, and their component nuclease, NLS and linker sequences are described in Sections 6.3, 6.4, and 6.5, respectively.

The disclosure further provides B-GEn Type V CRISPR Cas systems comprising engineered B-GEn polypeptides and suitable guide RNAs, or nucleic acids encoding them. Exemplary B-GEn Type V CRISPR Cas systems are disclosed in Section 6.6 and exemplary guide RNAs are disclosed in Section 6.7. In some embodiments, a B-GEn Type V CRISPR Cas system is a ribonucleoprotein (RNP) complex, comprising an engineered B-GEn polypeptide and a guide RNA. Ribonucleoprotein complexes are described in Section 6.8.

The present disclosure further provides nucleic acids encoding the engineered B-GEn polypeptides, for example expression vectors for engineered B-GEn polypeptides. Exemplary nucleic acids are disclosed in Section 6.9 and exemplary vectors are disclosed in Section 6.10.

In certain aspects, provided herein is a method of targeting, editing, modifying, or manipulating a target DNA at one or more locations in a cell or in vitro. The methods generally entail introducing a B-GEn Type V CRISPR Cas system into the cell or into the in vitro environment under conditions that are suitable for the engineered B-GEn polypeptide to make one or more nicks or cuts or base edits in the target DNA, wherein the engineered B-GEn polypeptide is directed to the target DNA by the guide RNA in its processed or unprocessed form. In some embodiments, an RNP of the disclosure (comprising an engineered B-GEn polypeptide and a guide RNA) is used to edit the genome of a cell. In some embodiments, methods of genomic DNA editing using an RNP comprise nucleofection of a target cell comprising the genomic DNA with the RNP and exposing the target cell to conditions under which gene editing occurs, for example by culturing the target cell under conditions suitable for genomic editing by the B-GEn polypeptide.

Additional features, advantages and applications of the engineered B-GEn polypeptides of the disclosure are more particularly described below.

### 5. BRIEF DESCRIPTION OF THE FIGURES

FIGS. 1A-1B are schematic illustrations of exemplary engineered B-GEn polypeptide construct configurations. FIG. 1A-1 is an engineered B-GEn construct with an NLS-B-GEn-NLS configuration that has a nuclear localization signal (NLS) located at its N-terminus and another NLS located at its C-terminus, each connected to the B-GEn nuclease sequence via a linker (L). FIG. 1A-2 is an engineered B-GEn polypeptide construct with a B-GEn-NLS configuration that has a linker and an NLS located at its C-terminus only. FIG. 1A-3 is an engineered B-GEn polypeptide construct with an NLS-B-GEn configuration that has a linker and an NLS located at its N-terminus only. FIG. 1A-4 is an engineered B-GEn polypeptide construct without an NLS on either terminus. FIG. 1B-1 is an engineered B-GEn polypeptide construct that has a single NLS sequence connected to the B-GEn nuclease sequence via a linker. FIG. 1B-2 is a B-GEn construct that has two NLS sequences at its C-terminus, each connected via a linker. FIG. 1B-3 is an engineered B-GEn polypeptide construct that has three NLS sequences at its C-terminus, each connected via a linker. FIG. 1B-4 is an engineered B-GEn polypeptide construct that has four NLS sequences at its C-terminus, each connected via a linker. In some embodiments, the NLS sequence flanking either or both ends of the B-GEn nuclease sequence is a Nucleoplasmin NLS, an SV40 NLS, a c-myc NLS or any other NLS sequence and may be separated by the B-GEn nuclease sequence via a linker. Each NLS domain can contain more than one NLS sequence, which can be identical or different. Moreover, NLSs flanking both termini of a B-GEn nuclease sequence can be identical or different NLSs. The term B-GEn generally refers to a type V RNA programmable endonuclease having a sequence as described in Section 6.3, including but not limited to SEQ NO:4 (B-GEn.1), SEQ ID NO:5 (B-GEn.1.2), SEQ ID NO:6 (B-GEn.2) and sequence variants thereof.

FIG. 2 is a cartoon illustration of a cell-based assay that can be used for nuclear localization signal (NLS) screening.

FIGS. 3A-3C display amino acid sequences of exemplary engineered B-GEn.2 polypeptide constructs with different NLS configurations. FIG. 3A is the amino acid sequence of a construct with an NLS-B-GEn.2-NLS configuration (SEQ ID NO:172), whereby the N-terminal NLS comprises the Nucleoplasmin NLS that is connected to the B-GEn.2 nuclease sequence via a linker, and the C-terminal NLS comprises the SV40 NLS that is connected to the B-GEn.2 nuclease sequence via another linker. FIG. 3B is the amino acid sequence of a construct with a B-GEn.2-NLS configuration (SEQ ID NO:2), whereby the NLS on the C-terminal comprises the SV40 NLS that is connected to the B-GEn.2 nuclease sequence via a linker. FIG. 3C is the amino acid sequence of a construct with an NLS-B-GEn.2 configuration (SEQ ID NO:173), whereby the NLS located on the N-terminal comprises the Nucleoplasmin NLS, which is connected to the B-GEn.2 nuclease sequence via a linker.

FIG. 4 is a cartoon illustration of an exemplary nuclease purification workflow utilized to purify the engineered B-GEn.2 polypeptide (and control) constructs with various NLS configurations, which is described in Section 8.3.

FIGS. 5A-5E show results of an example 2-step purification of engineered B-GEn.2 polypeptide (and control) constructs. FIG. 5A shows a series of Heparin Fast Flow (FF) chromatograms. The stars above the peaks indicate the elution positions of individual constructs. The peak marked with a single star corresponds to a B-GEn.2 polypeptide construct without an NLS, also referred to as B-GEn.2 with full NLS deletion (B-GEn.2-Full-del); the peak marked with a double star corresponds to a construct with a B-GEn.2-NLS configuration, also referred to as B-GEn.2 with N-terminus NLS deletion (B-GEn.2-Ndel); and the peak marked with a triple star corresponds to a construct with an NLS-B-GEn.2 configuration, also referred to as B-GEn.2 with C-terminus NLS deletion (B-GEn.2-Cdel). FIGS. 5B and 5C are images of stained SDS-polyacrylamide gels loaded with peak purification fractions seen in FIG. 5A, whereby the protein fractions that correspond to the peaks in FIG. 5A are marked with a single star for B-GEn-2-Full-del, with a double star for B-GEn.2-Ndel and with a triple star for B-GEn.2-Cdel. FIG. 5D is a chromatogram of Size Exclusion Chromatography (SEC) of the protein fraction under the peak marked with the single star in FIG. 5A. FIG. 5E is a stained SDS-polyacrylamide gel loaded with purification fractions in FIG. 5D, whereby the protein fraction that corresponds to the large monomeric peak in FIG. 5D is marked with the single star.

FIG. 6 is a cartoon illustration of the in cellula gene editing workflow which is described in Section 8.4.1 in detail.

FIG. 7 is a bar graph showing the results of B2M gene editing with 4.3 sgRNA in iPSCs, using Cpf1/Cas12a or engineered B-GEn.2 polypeptides (B-GEn.2 construct depicted in FIG. 3A and is capped with NLS on both its N- and C-termini; B-GEn.2 Ndel: B-GEn.2 construct depicted in FIG. 3B and; B-GEn.2 Cdel: B-GEn.2 construct depicted in FIG. 3C). WT is the control condition with no Cas proteins. The amount of RNP used is indicated underneath each protein. Y-axis represents percent gene editing.

FIGS. 8A-8B are bar graphs showing the results of albumin gene editing in iPSCs using B-GEn.2 variants. FIG. 8A shows the results of albumin gene editing in iPSCs with sgRNA v4.3*, whereas FIG. 8B shows the results of albumin gene editing in iPSCs with sgRNA v4.4*. WT refers to the control condition with no Cas proteins; B-GEn.2 is the construct depicted in FIG. 3A and is capped with NLS on both its N- and C-termini. B-GEn.2 Ndel is the B-GEn.2 construct depicted in FIG. 3B and B-Gen.2 Cdel is the B-GEn.2 construct depicted in FIG. 3C). The amount of RNP used is indicated underneath each protein. Y-axis represents percent gene editing. * denotes 2′-O-methylation of the last three nucleotides of the sgRNA 3′ end.

FIG. 9 is a bar graph depicting the results of albumin gene editing in HEK293-T cells using B-GEn.2 variants with sgRNA v4.3*. WT refers to the control condition with no Cas proteins; B-GEn.2 #1 and #2 are two preparations of the construct depicted in FIG. 3A and are capped with NLS on both its N- and C-termini. B-GEn.2 Ndel is the B-GEn.2 construct depicted in FIG. 3B and B-Gen.2 Cdel is the B-GEn.2 construct depicted in FIG. 3C. The amount of RNP used is indicated underneath each protein. Y-axis represents percent gene editing. * denotes 2′-O-methylation of the last three nucleotides of the sgRNA 3′ end.

### 6. DETAILED DESCRIPTION

### 6.1. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

B-GEn polypeptide: As used herein, the term “B-GEn polypeptide” refers to a polypeptide comprising the amino acid sequence of at least the nuclease domain of B-GEn.1 (SEQ ID NO:4, whose nuclease domain comprises RuvC I, RuvC II, and RuvC Ill sub-domains corresponding to amino acids 542-624, 825-876, and 956-970, respectively), B-GEn.1.2 (SEQ ID NO:5, whose nuclease domain comprises RuvC I, RuvC II, and RuvC Ill sub-domains corresponding to amino acids 537-621, 822-873, and 954-968, respectively), B-GEn.2 (SEQ ID NO:6, whose nuclease domain comprises RuvC I, RuvC II, and RuvC Ill sub-domains corresponding to amino acids 537-621, 822-873, and 954-968, respectively) or a variant thereof having at least 50% sequence identity to such nuclease domain. “B-GEn polypeptide” also encompasses a variant of any of B-GEn.1 (SEQ ID NO:4), B-GEn.1.2 (SEQ ID NO:5), B-GEn.2 (SEQ ID NO:6), such as a variant comprising an amino acid sequence having at least 50% sequence identity to SEQ ID NO:4, SEQ ID NO:5 or SEQ ID NO:6 and/or (2) a variant comprising an amino acid sequence that differs from SEQ ID NO:4, SEQ ID NO:5 or SEQ ID NO:6 by up to 25 amino acids. In some embodiments, the B-GEn polypeptide has nuclease activity. The term “B-GEn polypeptide” encompasses engineered fusion polypeptides that includes the amino acid sequence of B-GEn.1 (SEQ ID NO:4), B-GEn.1.2 (SEQ ID NO:5), B-GEn.2 (SEQ ID NO:6), or any variant thereof as described in Section 6.3, for example an amino acid sequence (1) having at least 50%, at least 60%, at least 70%, at least 80%, at least 90%, at least 95%, at least 96%, at least 97%, at least 98% sequence identity, at least 99% sequence identity, or 100% sequence identity to the nuclease domain or entire length of any of SEQ ID NOS:4 to 6 and/or (2) that differs from any one of SEQ ID NOS:4 to 6 by up to 25 amino acids, by up to 20 amino acids, by up to 15 amino acids, by up to 14 amino acids, by up to 13 amino acids, by up to 12 amino acids, by up to 11 amino acids, by up to 10 amino acids, by up to 9 amino acids, by up to 8 amino acids, by up to 7 amino acids, by up to 6 amino acids, or by up to 5 amino acids, together with additional sequences (e.g., one or more nuclear localization sequences as described in Section 6.3 and/or one or more linker sequences as described in Section 6.5). Exemplary engineered B-GEn polypeptides are described in Section 6.2.

Binding: As used herein, the term “binding” (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10-7 M, less than 10⁻⁸ M, less than 10-9 M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

Cell Therapy: As used herein, the term “cell therapy” refers to a therapy in which cellular material is administered to a patient. The cellular material may be intact, living cells. For example, T cells capable of fighting cancer cells via cell-mediated immunity may be administered in the course of immunotherapy. Cell therapy is also called cellular therapy or cytotherapy.

Coding Sequence: As used herein, the term “coding sequence” or “encoding nucleic acid” refers to a sequence within a nucleic acid (RNA or DNA) molecule that encodes a protein or RNA molecule. The coding sequence can further include initiation and termination signals operably linked to regulatory elements including a promoter and polyadenylation signal capable of directing expression in the cells of an individual or mammal to which the nucleic acid is introduced or administered. The coding sequence may be codon optimized for expression in a cell of interest.

Complement: As used herein, the terms “complement” and “complementary” in the context of a nucleic acid molecule refer to the ability to form Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

Encoding: The term “encoding” in relation to a nucleic acid (DNA or RNA) means that the nucleic acid comprises a nucleotide sequence coding for the amino acids of a polypeptide or the nucleotides of an RNA.

Expression Cassette: As used herein, the term “expression cassette” refers to a DNA coding sequence operably linked to a promoter.

Guide RNA: As used herein, the term “guide RNA” refers to a ribonucleic acid having a DNA-targeting sequence (also referred to as “spacer” or “DNA-targeting segment”) and a protein-binding sequence (also referred to as “protein-binding segment”). The DNA-targeting sequence has sufficient complementarity with a target DNA (e.g., genomic DNA) sequence, to hybridize with the target DNA sequence and direct sequence-specific binding of a nucleic acid-targeting complex to the target DNA sequence. The DNA-targeting sequence generally includes the “protospacer-like” sequence described herein. The protein-binding sequence interacts with a site-specific modifying enzyme (e.g., a B-GEn polypeptide as described in Sections 6.2 and 6.3 below). Site-specific cleavage of the target DNA occurs at locations determined by both (i) base pairing complementarity between the guide RNA and the target DNA; and (ii) a short motif (referred to as the protospacer adjacent motif (PAM)) in the target DNA. The protein-binding segment of a guide RNA includes, in part, two complementary stretches of nucleotides that hybridize to one another to form a double stranded RNA duplex (dsRNA duplex). In some embodiments, a guide RNA is a single-stranded guide RNA (sgRNA).

A guide RNA and a site-specific modifying enzyme such as a B-GEn polypeptide may form a ribonucleoprotein complex (e.g., bind via non-covalent interactions). The guide RNA provides target specificity to the complex by comprising a nucleotide sequence that is complementary to a sequence of a target DNA. The site-specific modifying enzyme of the complex provides the endonuclease activity. In other words, the site-specific modifying enzyme is guided to a target DNA sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

Heterologous: As used herein, the term “heterologous” refers to a nucleotide or peptide that is not found in the native nucleic acid or polypeptide, respectively. A B-GEn.1, or B-GEn.1.2, or B-GEn.2 fusion protein described herein may in some embodiments comprise the DNA- or RNA-binding domain of the B-GEn.1, or B-GEn.1.2, or B-GEn.2 polypeptide (or a variant thereof) fused to a heterologous polypeptide sequence (e.g., a polypeptide sequence from a protein other than B-GEn.1 or B-GEn.2). The heterologous polypeptide may exhibit an activity (e.g., enzymatic activity) that will also be exhibited by the B-GEn.1, or B-GEn.1.2, or B-GEn.2 fusion protein (e.g., methyltransferase activity, acetyltransferase activity, kinase activity, ubiquitinating activity, etc.). A heterologous nucleic acid may be linked to a naturally occurring nucleic acid (or a variant thereof) (e.g., by genetic engineering) to generate a fusion nucleic acid encoding a fusion polypeptide. As another example, in a fusion variant B-GEn.1, or B-GEn.1.2, or B-GEn.2 polypeptide, a variant B-GEn.1, or B-GEn.1.2, or B-GEn.2 polypeptide may be fused to a heterologous polypeptide (e.g., a polypeptide other than B-GEn.1 or B-GEn.2), which exhibits an activity that will also be exhibited by the fusion variant B-GEn.1, or B-GEn.1.2, or B-GEn.2 polypeptide. A heterologous nucleic acid may be linked to a variant B-GEn.1, or B-GEn.1.2, or B-GEn.2 polypeptide (e.g., by genetic engineering) to generate a nucleic acid encoding a fusion variant B-GEn.1, or B-GEn.1.2, or B-GEn.2 polypeptide. “Heterologous,” as used herein, additionally means a nucleotide or polypeptide in a cell that is not its native cell.

Host cell: As used herein, the terms “host cell” and “recombinant host cell” refer to a cell that has been genetically engineered, e.g., through introduction of a heterologous polypeptide or nucleic acid such as a vector or system of the disclosure. It should be understood that such terms are intended to refer not only to the particular subject cell but to the progeny of such a cell. In some embodiments, a host cell carries a vector of the disclosure as an extrachromosomal heterologous expression vector. In some embodiments, a host cell comprises any one of the engineered B-GEn polypeptides disclosed herein, e.g., as introduced as an RNP complex. In other embodiments, a host cell has undergone gene editing by an engineered B-GEn polypeptide of the disclosure.

iPSC: As used herein, the terms “induced pluripotent stem cell” and “iPSC” refer to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors. iPSCs can be derived from multiple different cell types, including terminally differentiated cells. iPSCs have an embryonic stem (ES) cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, Fox03, GDF3, Cyp26al, TERT, and zfp42.

Examples of methods of generating and characterizing iPSCs may be found in, for example, US Patent Publication Nos. US20090047263, US20090068742, US20090191159, US20090227032, US20090246875, and US20090304646 and PCT patent publications WO2013177133 and WO2022204567, the disclosures of each of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

Nucleic Acid: As used herein, the terms “nucleic acid,” “oligonucleotide” and “nucleic acid” refer to at least two nucleotides covalently linked together. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. The depiction of a single strand also defines the sequence of the complementary strand. Thus, reference to a single stranded nucleic acid herein also encompasses the complementary strand of a depicted single strand.

Nuclear Localization Signal: As used herein, the terms “nuclear localization signal” and “NLS” refer to an amino acid sequence that can facilitate the localization of a polypeptide to the nucleus of a eukaryotic cell.

Nuclease: As used herein, the terms “nuclease” and “endonuclease” are used interchangeably herein to mean an enzyme which possesses endonucleolytic catalytic activity for nucleic acid cleavage, as well as nuclease-inactivated variants thereof.

Nuclease Domain: As used herein, the terms “nuclease domain” and “cleavage domain” or “active domain” of a nuclease refer to the amino acid sequence or domain within the nuclease which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide. For example, the nuclease domains of B-GEn endonucleases comprise RuvC I, RuvC II, and RuvC Ill sub-domains corresponding to amino acids 542-624, 825-876, and 956-970, respectively, of SEQ ID NO:4 (B-GEn.1), amino acids 537-621, 822-873, and 954-968, respectively, of SEQ ID NO:5 (B-GEn.1.2), and amino acids 537-621, 822-873, and 954-968, respectively, of SEQ ID NO:6 (B-GEn.2). In some embodiments, the boundaries of a nuclease domain of a B-GEn polypeptide are determined by aligning the B-GEn polypeptide with AacC2C1 (Cas12b) (Uniprot #TOD7A2) and identifying the amino acids that align with the AacC2C1 (Cas12b) RuvC nuclease domain corresponding to amino acids R519 to S628. In the case of the B.GEn.2, the RuvC domain boundaries corresponding to amino acids R514 to S620, with D566 as active site residue. In other embodiments, the boundaries of a nuclease domain of a B-GEn polypeptide are determined by aligning the B-GEn polypeptide with BthCas12b (Wu et al., 2017, Cell Research 27:705-708) and identifying the amino acids that align with the BthCas12b RuvC nuclease domain comprising RuvC I, RuvC II, and RuvC Ill sub-domains.

Nucleofection: As used herein, the term “nucleofection” refers to an electroporation-based transfection method, which uses a combination of electrical parameters and cell-type specific reagents to transfer nucleic acids, such as DNA or RNA, and RNPs directly to the nuclei of target cells.

Operably linked: As used herein, the term “operably linked” refers to a functional relationship between two or more peptide or polypeptide domains or nucleic acid (e.g., DNA) segments. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, peptide and protein: As used herein, the terms “polypeptide,” “peptide” and “protein” refer to polymers of amino acids of any length. The polymer may in various embodiments be linear or branched, it may comprise modified amino acids, and it may be interrupted by non-amino acids.

Pluripotent: As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells derived from the inner cell mass of a blastocyst or derived by somatic cell nuclear transfer, and iPSCs derived from non-pluripotent cells.

Promoter: As used herein, the term “promoter” refers to a nucleotide sequence recognized by the synthetic machinery of the cell, or introduced synthetic machinery, required to initiate the specific transcription of a nucleic acid sequence. A promoter can be a constitutively active promoter (e.g., a promoter that is constitutively in an active “ON” state), it may be an inducible promoter (e.g., a promoter whose state, active/“ON” or inactive/“OFF”, is controlled by an external stimulus, e.g., the presence of a particular temperature, compound, or protein.), it may be a spatially restricted promoter (e.g., transcriptional control element, enhancer, etc.)(e.g., tissue specific promoter, cell type specific promoter, etc.), and it may be a temporally restricted promoter (e.g., the promoter is in the “ON” state or “OFF” state during specific stages of embryonic development or during specific stages of a biological process, e.g., hair follicle cycle in mice).

Protospacer Adjacent Motif: As used herein, the terms “protospacer adjacent motif” or “PAM” refer to a DNA sequence downstream (e.g., immediately downstream) of a target sequence on the non-target strand recognized by a Cas protein. A PAM sequence is located 3′ of the target sequence on the non-target strand.

Recombinant: As used herein, the term “recombinant” in relation to a nucleic acid, polypeptide or cell refers to a nucleic acid (DNA or RNA), polypeptide or cell that is the product of genetic engineering, either directly or indirectly (e.g., is the progeny or replica of a nucleic acid, polypeptide or cell generated by genetic engineering methods). For example, a recombinant vector can be the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may indeed act to modulate production of a desired product by various mechanisms (see “DNA regulatory sequences”, below). In addition or alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is generally done to replace a codon with a codon encoding the same amino acid, a conservative amino acid, or a non-conservative amino acid. In addition or alternatively, it is performed to join together nuclei acid segments of desired functions to generate a desired combination of functions. This artificial combination is often accomplished by either chemical synthesis means, or by the artificial manipulation of isolated segments of nucleic acids, e.g., by genetic engineering techniques. When a recombinant nucleic acid encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention but may be a naturally occurring amino acid sequence. The term “non-naturally occurring” includes molecules that are markedly different from their naturally occurring counterparts, including chemically modified or mutated molecules.

Regulatory sequence: As used herein, the term “regulatory sequence” refers to a nucleic acid sequence which is required for expression of an operably linked sequence of interest, e.g., a guide RNA or an engineered B-GEn polypeptide sequence. In some instances, the regulatory sequence may be a promoter sequence and in other instances, the regulatory sequence may include a promoter and an enhancer sequence and/or other regulatory elements which are required for expression of the pol. The regulatory sequence may, for example, be one which drives the expression of the operably linked sequence constitutively or in a tissue specific manner.

Ribonucleoprotein (RNP) complex, ribonucleoprotein (RNP) particle: As used herein, the terms “ribonucleoprotein complex” and “ribonucleoprotein particle” refer to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid may be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. In some embodiments, any one of the engineered B-GEn polypeptides disclosed herein is in an RNP with a guide RNA.

Spacer: As used herein, the term “spacer” refers to a region of a gRNA molecule which is partially or fully complementary to a target sequence found in the + or − strand of genomic DNA. When complexed with a Gas protein, the gRNA directs the Gas protein to the target sequence in the genomic DNA. A spacer is typically 15 to 30 nucleotides in length (e.g., 20-25 nucleotides). The nucleotide sequence of a spacer can be, but is not necessarily, fully complementary to the target sequence. For example, in some embodiments, a spacer can contain one or more mismatches with a target sequence, e.g., the spacer can comprise one, two, or three mismatches with the target sequence.

Stem-Loop Structure: As used herein, the term “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, e.g., not include any mismatches.

Target cell: As used herein, the term “target cell” refers to a cell into which a nuclease, e.g., a B-GEn system of the disclosure, is introduced, for example a cell comprising target DNA in its genome. It should be understood that such term is intended to refer not only to the particular subject cell but to the progeny of such a cell. Because gene editing can take place in the cell as a result of the nuclease system, such progeny need not be identical to the parent cell into which the system was initially introduced but include gene edited counterparts of the cell. Such gene edited progeny are still included within the scope of the term “target cell” as used herein.

Target DNA: As used herein, the term “target DNA” refers to a polydeoxyribonucleotide that includes a “target site” or “target sequence.” The terms “target site,” “target sequence,” “target protospacer DNA,” or “protospacer-like sequence” are used interchangeably herein to refer to a nucleic acid sequence present in a target DNA to which a DNA-targeting segment (also referred to as a “spacer”) of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, J. and Russell, W., 2001. Molecular Cloning: A Laboratory Manual, Third Edition, Cold Spring Harbor Laboratory Press. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand” or “non-complementary strand.” In some embodiments, the target DNA is genomic DNA.

Transfection: As used herein, the term “transfection” refers to the introduction of nucleic acid molecules, such as DNA or RNA (e.g., mRNA) molecules, into cells, e.g., into nuclei of target or production cells. In the context of the present disclosure, the term “transfection” encompasses any method known to the skilled person for introducing nucleic acid molecules into cells, e.g., into eukaryotic cells, such as into mammalian cells. Such methods encompass, for example, electroporation, lipofection, e.g., based on cationic lipids and/or liposomes, calcium phosphate precipitation, nanoparticle-based transfection, virus-based transfection, or transfection based on cationic polymers, such as DEAE-dextran or polyethylenimine.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of nucleotide sequences to which they are operably linked. Such vectors are referred to herein as “expression vectors”. In some embodiments, a vector is a viral vector, e.g., an adenoviral vector, or an adeno-associated virus (AAV) vector.

### 6.2. Engineered B-GEn Endonucleases

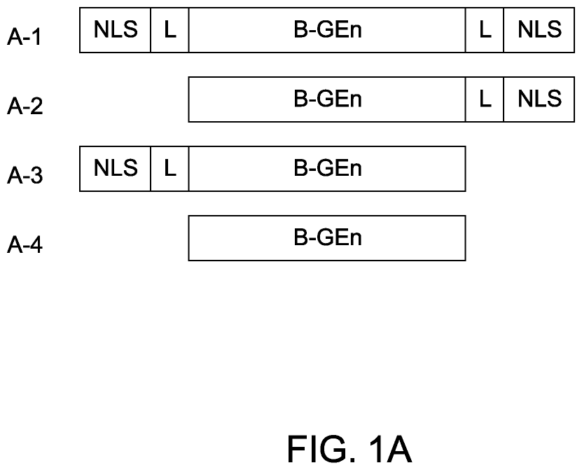

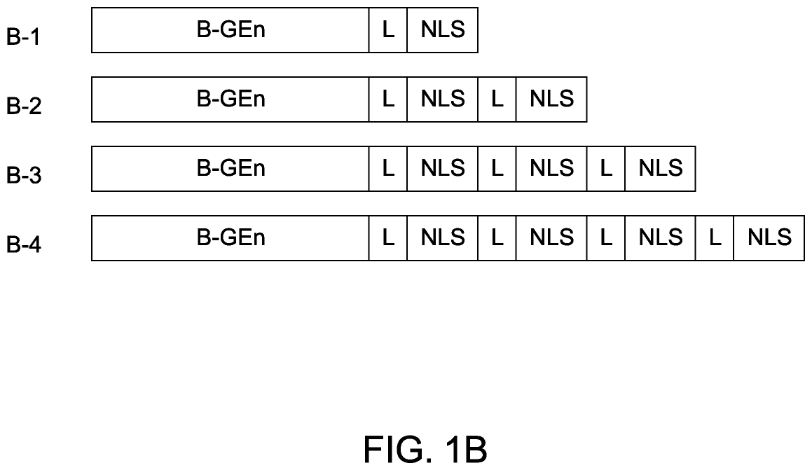

The present disclosure relates to engineered Type V CRISPR-Cas endonucleases comprising the following:
(i) a nuclease sequence, such as a B-GEn polypeptide sequence, as described in Section 6.3;
(ii) one or more NLS sequences as described in Section 6.4; and
(iii) optional linkers as described in Section 0.
   Exemplary configurations of engineered Type V CRISPR-Cas endonucleases are set forth in FIGS. 1A and 1B.

In certain aspects, an engineered Type V CRISPR-Cas endonuclease comprises a nuclease sequence and a first NLS sequence C-terminal to the nuclease sequence. An engineered Type V CRISPR-Cas endonuclease comprising a nuclease sequence and a first NLS sequence can further comprise a first linker sequence between the nuclease sequence and the first NLS sequence. An exemplary configuration is shown in FIG. 1B, B-1.

In certain aspects, an engineered Type V CRISPR-Cas endonuclease comprises more than one NLS sequence (e.g., more than one NLS sequences C-terminal to the nuclease sequence).

In some embodiments, an engineered Type V CRISPR-Cas endonuclease comprises a second NLS sequence C-terminal to the first NLS sequence. An engineered Type V CRISPR-Cas endonuclease comprising a second NLS sequence can further comprise a linker sequence between the first NLS sequence and the second NLS sequence. An exemplary configuration is shown in FIG. 1B, B-2.

In further embodiments, an engineered Type V CRISPR-Cas endonuclease comprises a third NLS sequence C-terminal to the second NLS sequence. An engineered Type V CRISPR-Cas endonuclease comprising a third NLS sequence can further comprise a linker sequence between the second NLS sequence and the third NLS sequence. An exemplary configuration is shown in FIG. 1B, B-3.

In additional embodiments, an engineered Type V CRISPR-Cas endonuclease comprises a fourth NLS sequence C-terminal to the third NLS sequence. An engineered Type V CRISPR-Cas endonuclease comprising a fourth NLS sequence can further comprise a linker sequence between the third NLS sequence and the fourth NLS sequence. An exemplary configuration is shown in FIG. 1B, B-4.

In some embodiments, an engineered Type V CRISPR-Cas endonuclease comprises an N-terminal NLS sequence in addition to the one or more NLS sequences C-terminal to the nuclease sequence. Thus, in certain aspects, an engineered Type V CRISPR-Cas endonuclease comprises a NLS sequence N-terminal to the nuclease sequence. An engineered Type V CRISPR-Cas endonuclease comprising a NLS sequence N-terminal to the nuclease sequence can further comprise a linker sequence between the NLS sequence nuclease sequence. Exemplary configurations are shown in FIGS. 1A, A1 and A3. In certain embodiments, an engineered Type V CRISPR-Cas endonuclease comprises more than one N-terminal NLS sequence (e.g., more than one NLS sequence N-terminal to the nuclease sequence that may be connected via one or more linkers).

Exemplary amino acid sequences of engineered Type V CRISPR-Cas endonucleases are set forth in Table 1. In some embodiments, an engineered Type V CRISPR-Cas endonuclease comprises the amino acid sequence of SEQ ID NO:1. In some embodiments, an engineered Type V CRISPR-Cas endonuclease comprises the amino acid sequence of SEQ ID NO:2. In some other embodiments, an engineered an engineered Type V CRISPR-Cas endonuclease comprises the amino acid sequence of SEQ ID NO:3.

TABLE 1

| Engineered | | SEQ ID |
|---|---|---|
| Endonuclease | Amino Acid Sequence | NO |
| His-tag- | MGSSHHHHHHHMKRPAATKKAGQAKKKKGGGGSGGG | 1 |
| Nucleoplasmin | GSGGGGSGSMPIRSFKLKLVTHNGDSTYMDKLRRGLWK | |
| NLS-Linker- | THVIINRGIAYYMNTLALMRQEPYGSKSREEVRLDLLSTLR | |
| B-GEn.2- | EQQRRNNWSEQTGTDDELLSLSRRVYELLVPSAIGEKGD | |
| Linker-SV40 | AQMLSRKFLSPLVDPNSEGGRGTAKSGRKPRWKKMMEE | |
| NLS aa | GHPDWEKEKEKDAAKKAEDPTASILADLEAVGLLPLFPLF | |
| sequence | SDEQKEIRWLPKKKRQFVRTWDRDMFQQALERMLSWES | |
| | WNRRVAEEYLKLQAQRDEVYAKYLEDAGSWLNDLQTFEK | |
| | QREEELAEVSFEPNSEYLITRRQIRGWKEVYEKWSKTSEN | |
| | ASQEQLWRMVADVQTAMAGAFGDPKVYQFLSQPKHHHI | |
| | WREHPNRLFYYSKYNEVREKLNRAKKQAAFTLPDPVEHP | |
| | LWTRFDARGGNIHDYEISKVGKQYHVTFSSLILPEAQSWV | |
| | EIENVTVGIGNSLQLKRQIRLDGYADKKQKVKYYDYSSRF | |
| | ELTGVLGGAKIQFDRKHLKKAAHRLAEGETGPIFLNVVVD | |
| | VEPFLEVKNGRLRTPLGQVLQVNTRDWPKVVDYKAKELS | |
| | VLMENTQIGNENGVSTIEAGMRIMSIDLGQRTAAAVSIFEVI | |
| | SKKPDEKETKLFYPIADTDLYAVHRRSLLLRLPGEEISSKK | |
| | MIEKRKERARIRSLVRYQIRLLSEVLRLHTQGTAEQRRFKL | |
| | DELLVSIQKKLELDQSEWISELEKLFDYIDESAEKWKEALV | |
| | VAHRTLEPIVVEAVRNWKKSLSKENKDRRRIAGISIWSIEE | |
| | LEETRKLLIAWSKHSREPGIPKRLEKEETFAPEHLQHIQNV | |
| | KDDRLKQMANLFVMTALGYKYDEGNKRWVEAYPACQVIL | |
| | FEDLSRYRFALDRPRRENNRLMKWAHRSIPRLTYMQAEL | |
| | FGIQVGDVYSAYTSRFHAKTGAPGIRCHALTEADLQSNSY | |
| | VVNQLIKDKFIQDNQTEILKAGQIVPWQGGELFVTFADRSG | |
| | ASLAVIHADINAAQNLQKRFWQHNSEVFRVPCKVVKGGLV | |
| | PVYEKMRKLFGKGLFVNIDDPESKEVYRWEHSTKMKSKT | |
| | TPVDLESEDIDHEELSDEWEDMQEGYKTLLRDPSGFFWS | |
| | SDSWIPQKDFWIRVKSRIGKSLREQIRGGSGGSGGSGPP | |
| | KKKRKV | |
| B-GEn.2- | MPIRSFKLKLVTHNGDSTYMDKLRRGLWKTHVIINRGIAY | 2 |
| Linker-SV40 | YMNTLALMRQEPYGSKSREEVRLDLLSTLREQQRRNNWS | |
| NLS aa | EQTGTDDELLSLSRRVYELLVPSAIGEKGDAQMLSRKFLS | |
| sequence | PLVDPNSEGGRGTAKSGRKPRWKKMMEEGHPDWEKEK | |
| | EKDAAKKAEDPTASILADLEAVGLLPLFPLFSDEQKEIRWL | |
| | PKKKRQFVRTWDRDMFQQALERMLSWESWNRRVAEEY | |
| | LKLQAQRDEVYAKYLEDAGSWLNDLQTFEKQREEELAEV | |
| | SFEPNSEYLITRRQIRGWKEVYEKWSKTSENASQEQLWR | |
| | MVADVQTAMAGAFGDPKVYQFLSQPKHHHIWREHPNRL | |
| | FYYSKYNEVREKLNRAKKQAAFTLPDPVEHPLWTRFDAR | |
| | GGNIHDYEISKVGKQYHVTFSSLILPEAQSWVEIENVTVGI | |
| | GNSLQLKRQIRLDGYADKKQKVKYYDYSSRFELTGVLGG | |
| | AKIQFDRKHLKKAAHRLAEGETGPIFLNVVVDVEPFLEVKN | |
| | GRLRTPLGQVLQVNTRDWPKVVDYKAKELSVLMENTQIG | |
| | NENGVSTIEAGMRIMSIDLGQRTAAAVSIFEVISKKPDEKE | |
| | TKLFYPIADTDLYAVHRRSLLLRLPGEEISSKKMIEKRKERA | |
| | RIRSLVRYQIRLLSEVLRLHTQGTAEQRRFKLDELLVSIQK | |
| | KLELDQSEWISELEKLFDYIDESAEKWKEALVVAHRTLEPI | |
| | VVEAVRNWKKSLSKENKDRRRIAGISIWSIEELEETRKLLIA | |
| | WSKHSREPGIPKRLEKEETFAPEHLQHIQNVKDDRLKQM | |
| | ANLFVMTALGYKYDEGNKRWVEAYPACQVILFEDLSRYR | |
| | FALDRPRRENNRLMKWAHRSIPRLTYMQAELFGIQVGDV | |
| | YSAYTSRFHAKTGAPGIRCHALTEADLQSNSYVVNQLIKD | |
| | KFIQDNQTEILKAGQIVPWQGGELFVTFADRSGASLAVIHA | |
| | DINAAQNLQKRFWQHNSEVFRVPCKVVKGGLVPVYEKMR | |
| | KLFGKGLFVNIDDPESKEVYRWEHSTKMKSKTTPVDLESE | |
| | DIDHEELSDEWEDMQEGYKTLLRDPSGFFWSSDSWIPQK | |
| | DFWIRVKSRIGKSLREQIRGGSGGSGGSGPPKKKRKV | |
| His-tag- | MGSSHHHHHHHMKRPAATKKAGQAKKKKGGGGSGGGGS | 3 |
| Nucleoplasmin | GGGGSGSMPIRSFKLKLVTHNGDSTYMDKLRRGLWKTH | |
| NLS-Linker- | VIINRGIAYYMNTLALMRQEPYGSKSREEVRLDLLSTLR | |
| B-GEn.2 aa | EQQRRNNWSEQTGTDDELLSLSRRVYELLVPSAIGEKGD | |
| sequence | AQMLSRKFLSPLVDPNSEGGRGTAKSGRKPRWKKMMEE | |
| | GHPDWEKEKEKDAAKKAEDPTASILADLEAVGLLPLFPLF | |
| | SDEQKEIRWLPKKKRQFVRTWDRDMFQQALERMLSWES | |
| | WNRRVAEEYLKLQAQRDEVYAKYLEDAGSWLNDLQTFEK | |
| | QREEELAEVSFEPNSEYLITRRQIRGWKEVYEKWSKTSEN | |
| | ASQEQLWRMVADVQTAMAGAFGDPKVYQFLSQPKHHHI | |
| | WREHPNRLFYYSKYNEVREKLNRAKKQAAFTLPDPVEHP | |
| | LWTRFDARGGNIHDYEISKVGKQYHVTFSSLILPEAQSWV | |
| | EIENVTVGIGNSLQLKRQIRLDGYADKKQKVKYYDYSSRF | |
| | ELTGVLGGAKIQFDRKHLKKAAHRLAEGETGPIFLNVVVD | |
| | VEPFLEVKNGRLRTPLGQVLQVNTRDWPKVVDYKAKELSV | |
| | LMENTQIGNENGVSTIEAGMRIMSIDLGQRTAAAVSIFEVI | |
| | SKKPDEKETKLFYPIADTDLYAVHRRSLLLRLPGEEISSKK | |
| | MIEKRKERARIRSLVRYQIRLLSEVLRLHTQGTAEQRRFKL | |
| | DELLVSIQKKLELDQSEWISELEKLFDYIDESAEKWKEALV | |
| | VAHRTLEPIVVEAVRNWKKSLSKENKDRRRIAGISIWSIEE | |
| | LEETRKLLIAWSKHSREPGIPKRLEKEETFAPEHLQHIQNV | |
| | KDDRLKQMANLFVMTALGYKYDEGNKRWVEAYPACQVIL | |
| | FEDLSRYRFALDRPRRENNRLMKWAHRSIPRLTYMQAEL | |
| | FGIQVGDVYSAYTSRFHAKTGAPGIRCHALTEADLQSNSY | |
| | VVNQLIKDKFIQDNQTEILKAGQIVPWQGGELFVTFADRSG | |
| | ASLAVIHADINAAQNLQKRFWQHNSEVFRVPCKVVKGGLV | |
| | PVYEKMRKLFGKGLFVNIDDPESKEVYRWEHSTKMKSKT | |
| | TPVDLESEDIDHEELSDEWEDMQEGYKTLLRDPSGFFWS | |
| | SDSWIPQKDFWIRVKSRIGKSLREQIR | |

In one embodiment, a B-GEn fusion protein (SEQ ID NO: 1) comprises a Nucleoplasmin NLS (SEQ ID NO:8) on the N-terminus and an SV40 large T protein NLS (SEQ ID NO:7) on the C-terminus of a B-GEn.2 sequence (SEQ ID NO:6).

In another embodiment, a B-GEn fusion protein (SEQ ID NO: 2) comprises an SV40 large T protein NLS (SEQ ID NO:7) on the C-terminus of a B-GEn.2 sequence (SEQ ID NO:6).

In another embodiment, a B-GEn fusion protein (SEQ ID NO: 3) comprises a Nucleoplasmin NLS (SEQ ID NO:8) on the N-terminus of a B-GEn.2 sequence (SEQ ID NO:6).

### 6.3. The Nuclease Sequence

The present disclosure provides engineered Type V CRISPR-Cas endonucleases comprising, inter alia, a B-GEn nuclease amino acid sequence.

The engineered B-GEn polypeptides of the disclosure typically comprise an amino acid sequence that is at least 50% identical to the nuclease domain (e.g., an amino acid sequence composed of the RuvC I, RuvC II, and RuvC Ill sub-domains) or entire length of SEQ ID NO:4, or SEQ ID NO:5 or SEQ ID NO:6 and/or differs from SEQ ID NO:4, SEQ ID NO:5 or SEQ ID NO:6 by up to 25 amino acids. In various embodiments, an engineered B-GEn polypeptide of the disclosure comprises an amino acid sequence that is at least 55%, at least 60%, at least 65%, at least 70%, at least 75%, at least 85%, at least 90%, or at least 95% identical to the nuclease domain (e.g., an amino acid sequence composed of the RuvC I, RuvC II, and RuvC Ill sub-domains) or entire length of SEQ ID NO:4, or SEQ ID NO:5 or SEQ ID NO:6. In some embodiments, an engineered B-GEn polypeptide comprises an amino acid sequence that is at least 95%, at least 96%, at least 97%, at least 98%, at least 99% identical or at least 99.5% identical to the nuclease domain (e.g., an amino acid sequence composed of the RuvC I, RuvC II, and RuvC Ill sub-domains) or entire length of SEQ ID NO:4 or SEQ ID NO:5 or SEQ ID NO:6. In some embodiments, an engineered B-GEn polypeptide comprises an amino acid sequence that is identical to the nuclease domain or entire length of SEQ ID NO:4 or SEQ ID NO:5 or SEQ ID NO:6.

In further aspects, an engineered B-GEn polypeptide of the disclosure may comprise an amino acid sequence that differs from the nuclease domain sequence (e.g., the amino acid sequence composed of the RuvC I, RuvC II, and RuvC Ill sub-domains) of SEQ ID NO:4, SEQ ID NO:5 or SEQ ID NO:6 by up to 25 amino acids. In some embodiments, an engineered B-GEn polypeptide comprises an amino acid sequence that differs from the nuclease domain sequence (e.g., the amino acid sequence composed of the RuvC I, RuvC II, and RuvC III sub-domains) of any one of SEQ ID NOS:4 to 6 by up to 25 amino acids, by up to 20 amino acids, by up to 15 amino acids, by up to 14 amino acids, by up to 13 amino acids, by up to 11 amino acids, by up to 10 amino acids, by up to 9 amino acids, by up to 8 amino acids, by up to 7 amino acids, by up to 6 amino acids, or by up to 5 amino acids.

In yet further aspects, an engineered B-GEn polypeptide of the disclosure may comprise an amino acid sequence that differs from the entire sequence of SEQ ID NO:4, SEQ ID NO:5 or SEQ ID NO:6 by up to 25 amino acids. In some embodiments, an engineered B-GEn polypeptide differs comprises an amino acid sequence that differs from the entire length of any one of SEQ ID NOS:4 to 6 by up to 25 amino acids, by up to 20 amino acids, by up to 15 amino acids, by up to 14 amino acids, by up to 13 amino acids, by up to 12 amino acids, by up to 11 amino acids, by up to 10 amino acids, by up to 9 amino acids, by up to 8 amino acids, by up to 7 amino acids, by up to 6 amino acids, or by up to 5 amino acids.

Exemplary B-GEn.1, B-GEn.1.2 and B-GEn.2 nuclease sequences are set forth in Table 2.

TABLE 2

| | | SEQ ID |
|---|---|---|
| Name | Sequence | NO |
| B-GEn. 1 aa | MTIRSMKLKLKIYSGRSAPQLRQGLWRLHRLLNEGTAYYMDWLVHMRQE | 4 |
| sequence | ALPGKSKEEIRAELERRVRQQQEKNGVQNDQVPMDEVLSALRQLYELLV | |
| | PSAVNNSGDAQTLSRKFLSPLVDPNSEGGKGTSNAGAKPGWRKKQEAGD | |
| | PSWEKDYERWLKRKQADPTAEILGKLETAGLKPLFPLYTNEVKDIRWMP | |
| | LTSKQYVRNWDRDMFQQAIEHLLSWETWNRKVNEERAKLKETVRRFEEQ | |
| | HLANGKDWLSPLQAYEANREQALRDMAISPSDRFRITRRQIKGWSELYE | |
| | RWNKLAPTASVEAYMQEVRHVQKKLGGTFGDADLYRFLAKPENVHIWRD | |
| | HQERLHYYAAYNDLHKRLMSAKEQAAFTLPDPVAHPLWVREDARDGNLF | |
| | TYILQADSSKQRSRRYVNFSRFLWPVEDGYFEETENVKVELALSKQFYR | |
| | QVIVHDNPTGKQKITFQDYSSKEILEGHLGGAKLQLDRNFLRKSGRDFE | |
| | TGDFGPAFLNVVLDLKPKQEVKNGRLQSPLGQALLVKSRPNDIPKVYGY | |
| | KPDALAAWLEQASGEESLGSESLRQGFRVMSIDLGVRSAAAISVESVKG | |
| | EKTREGDKVCYPVGETGLFAVHDRSELLRLPGESSEKRVNVERDKRKTE | |
| | RMQIRYHIRTLARVLRLANKATPMDRIKAVQDVLNDIESTREMNDHDHH | |
| | VYNHALETLRTYAPDHQGIWEEQVIAAHRQLEHHVGVIVGEWRKNWGKD | |
| | RRGTVGLSMDNIEELDEMRRLLISWSRRARYPREAKPFQVNESNPVHLL | |
| | RHLQNLKEDRLKQLANLIVMTALGYVYDSKEKKWKAAYPACQLILFEDL | |
| | QRYRFHLDRSARENSQLMKWAHRSIPKYVWMQGEPYGLQIGDVWAGFTS | |
| | RYHAKTGAPGIRCKALTEKDFQQGRLLESLVAEGMFTLQEVGTLKPGDI | |
| | VPAEGGELFVTLADDSGDRIVITHADINAAQNVQKREWLANSERERVAC | |
| | RSVQIASQECFIPSSESVAKKMGKGVFVRDFSFHKDMEVYHWNNQVKLT | |
| | AKNVPTDHSDDLQDLQDYQAILEEARESSSSYKTLFRDPSGFFFPDDVW | |
| | VPQNIYWREVKKTITALLRKRIMST | |
| B-GEn. 1.2 aa | MPIRSFKLKLVTHNGDSTYMDKLRRGLWKTHVIINRGIAYYMNTLALMR | 5 |
| sequence | QEPYGSKSREEVRLDLLSTLREQQRRNNWSEQTGTDDELLSLSRRVYEL | |
| | LVPSAIGEKGDAQMLSRKFLSPLVDPNSEGGRGTAKSGRKPRWKKMMEE | |
| | GHPDWEKEKEKDAAKKAEDPTASILADLEAVGLLPLFPLESDEQKEIRW | |
| | LPKKKRQFVRTWDRDMFQQALERMLSWESWNRRVAEEYQKLQAQRDEVY | |
| | AKYLEDAGSWLNDLQTFEKQREEELAEVSFEPNSEYLITRRQIRGWKEV | |
| | YEKWSKTSENASQEQLWRMVADVQTAMAGAFGDPKVYQFLSQPKHHHIW | |
| | REHPNRLFYYSKYNEVREKLNRAKKQAAFTLPDPVEHPLWTRFDARGGN | |
| | IHDYEISKVGKQYHVTFSSLILPEAQSWVEIENVTVGIGNSLQLKRQIR | |
| | LDGYADKKQKVKYYDYSSRFELTGVLGGAKIQFDRKHLKKAAHRLAEGE | |
| | TGPIFLNVVVDVEPFLEVKNGRLRTPLGQVLQVNTRDWPKVVDYKAKEL | |
| | SVLMENTQIGNENGVSTIEAGMRIMSIDLGQRTAAAVSIFEVISKKPDE | |
| | KETKLFYPIADTDLYAVHRRSLLLRLPGEEISSKKMIEKRKERARIRSL | |
| | VRYQIRLLSEVLRLHTQGTAEQRRFKLDELLVSIQRKLELDQSEWISEL | |
| | EKLFDYIDESAEKWKEALVVAHRTLEPIVVEAVRNWKKSLSKENKDRRR | |
| | IAGISIWSIEELEETRKLLIAWSKHSREPGIPKRLEKEETFAPEHLQHI | |
| | QNVKDDRLKQMANLFVMTALGYKYDEGNKRWVEAYPACQVILFEDLSRY | |
| | RFALDRPRRENNRLMKWAHRSIPRLTYMQAELFGIQVGDVYSAYTSRFH | |
| | AKTGAPGIRCHALTEADLQSNSYVVNQLIKDKFIQDNQTEILKAGQIVP | |
| | WQGGELFVTFADRSGASLAVIHADINAAQNLQKRFWQHNSEVFRVPCKV | |
| | VKGGLVPVYEKMRKLFGKGLFVNIDDPESKEVYRWEHSTKMKSKTTPVD | |
| | LESEDIEHEELSDEWEDMQEGYKTLLRDPSGFFWSSDSWIPQKDFWIRV | |
| | KSRIGKSLREQIR | |
| B-GEn.2 aa | MPIRSFKLKLVTHNGDSTYMDKLRRGLWKTHVIINRGIAYYMNTLALMR | 6 |
| sequence | QEPYGSKSREEVRLDLLSTLREQQRRNNWSEQTGTDDELLSLSRRVYEL | |
| | LVPSAIGEKGDAQMLSRKFLSPLVDPNSEGGRGTAKSGRKPRWKKMMEE | |
| | GHPDWEKEKEKDAAKKAEDPTASILADLEAVGLLPLFPLFSDEQKEIRW | |
| | LPKKKRQFVRTWDRDMFQQALERMLSWESWNRRVAEEYLKLQAQRDEVY | |
| | AKYLEDAGSWLNDLQTFEKQREEELAEVSFEPNSEYLITRRQIRGWKEV | |
| | YEKWSKTSENASQEQLWRMVADVQTAMAGAFGDPKVYQFLSQPKHHHIW | |
| | REHPNRLFYYSKYNEVREKLNRAKKQAAFTLPDPVEHPLWTRFDARGGN | |
| | IHDYEISKVGKQYHVTFSSLILPEAQSWVEIENVTVGIGNSLQLKRQIR | |
| | LDGYADKKQKVKYYDYSSRFELTGVLGGAKIQFDRKHLKKAAHRLAEGE | |
| | TGPIFLNVVVDVEPFLEVKNGRLRTPLGQVLQVNTRDWPKVVDYKAKEL | |
| | SVLMENTQIGNENGVSTIEAGMRIMSIDLGQRTAAAVSIFEVISKKPDE | |
| | KETKLFYPIADTDLYAVHRRSLLLRLPGEEISSKKMIEKRKERARIRSL | |
| | VRYQIRLLSEVLRLHTQGTAEQRRFKLDELLVSIQKKLELDQSEWISEL | |
| | EKLFDYIDESAEKWKEALVVAHRTLEPIVVEAVRNWKKSLSKENKDRRR | |
| | IAGISIWSIEELEETRKLLIAWSKHSREPGIPKRLEKEETFAPEHLQHI | |
| | QNVKDDRLKQMANLFVMTALGYKYDEGNKRWVEAYPACQVILFEDLSRY | |
| | REALDRPRRENNRLMKWAHRSIPRLTYMQAELFGIQVGDVYSAYTSRFH | |
| | AKTGAPGIRCHALTEADLQSNSYVVNQLIKDKFIQDNQTEILKAGQIVP | |
| | WQGGELFVTFADRSGASLAVIHADINAAQNLQKRFWQHNSEVERVPCKV | |
| | VKGGLVPVYEKMRKLFGKGLFVNIDDPESKEVYRWEHSTKMKSKTTPVD | |
| | LESEDIDHEELSDEWEDMQEGYKTLLRDPSGFFWSSDSWIPQKDFWIRV | |
| | KSRIGKSLREQIR | |

One embodiment according to the disclosure are polypeptides comprising nuclease sequences with at least 80% sequence identity to any one of SEQ ID NO:4, SEQ ID NO:5, or SEQ ID NO:6, or nucleic acids comprising nucleotide sequences encoding polypeptides comprising nuclease sequences with least 80% sequence identity to any one of SEQ ID NO:4, SEQ ID NO:5, or SEQ ID NO:6

Another embodiment according to the disclosure are polypeptides comprising nuclease sequences with at least 85% sequence identity to any one of SEQ ID NO:4, SEQ ID NO:5, or SEQ ID NO:6, or nucleic acids comprising nucleotide sequences encoding polypeptides comprising nuclease sequences with least 85% sequence identity to any one of SEQ ID NO:4, SEQ ID NO:5, or SEQ ID NO:6

Yet another embodiment according to the disclosure are polypeptides comprising nuclease sequences with at least 90% sequence identity to any one of SEQ ID NO:4, SEQ ID NO:5, or SEQ ID NO:6, or nucleic acids comprising nucleotide sequences encoding polypeptides comprising nuclease sequences with least 90% sequence identity to any one of SEQ ID NO:4, SEQ ID NO:5, or SEQ ID NO:6

A further embodiment according to the disclosure are polypeptides comprising nuclease sequences with at least 95% sequence identity to any one of SEQ ID NO:4, SEQ ID NO:5, or SEQ ID NO:6, or nucleic acids comprising nucleotide sequences encoding polypeptides comprising nuclease sequences with least 95% sequence identity to any one of SEQ ID NO:4, SEQ ID NO:5, or SEQ ID NO:6

A yet further embodiment according to the disclosure are polypeptides comprising nuclease sequences with at least 96% sequence identity to any one of SEQ ID NO:4, SEQ ID NO:5, or SEQ ID NO:6, or nucleic acids comprising nucleotide sequences encoding polypeptides comprising nuclease sequences with least 96% sequence identity to any one of SEQ ID NO:4, SEQ ID NO:5, or SEQ ID NO:6

An additional embodiment according to the disclosure are polypeptides comprising nuclease sequences with at least 97% sequence identity to any one of SEQ ID NO:4, SEQ ID NO:5, or SEQ ID NO:6, or nucleic acids comprising nucleotide sequences encoding polypeptides comprising nuclease sequences with least 97% sequence identity to any one of SEQ ID NO:4, SEQ ID NO:5, or SEQ ID NO:6.

Another additional embodiment according to the disclosure are polypeptides comprising nuclease sequences with at least 98% sequence identity to any one of SEQ ID NO:4, SEQ ID NO:5, or SEQ ID NO:6, or nucleic acids comprising nucleotide sequences encoding polypeptides comprising nuclease sequences with least 98% sequence identity to any one of SEQ ID NO:4, SEQ ID NO:5, or SEQ ID NO:6.

Yet another embodiment according to the disclosure are polypeptides comprising nuclease sequences with at least 99% sequence identity to any one of SEQ ID NO:4, SEQ ID NO:5, or SEQ ID NO:6, or nucleic acids comprising nucleotide sequences encoding polypeptides comprising nuclease sequences with least 99% sequence identity to any one of SEQ ID NO:4, SEQ ID NO:5, or SEQ ID NO:6.

Yet another embodiment according to the disclosure are polypeptides comprising nuclease sequences with at least 99.5% sequence identity to any one of SEQ ID NO:4, SEQ ID NO:5, or SEQ ID NO:6, or nucleic acids comprising nucleotide sequences encoding polypeptides comprising nuclease sequences with least 99.5% sequence identity to any one of SEQ ID NO:4, SEQ ID NO:5, or SEQ ID NO:6.

### 6.4. The Nuclear Localization Signal

The disclosure provides engineered Type V CRISPR-Cas endonucleases, which are in the form of fusion proteins comprising a B-GEn nuclease sequence fused with one or more additional amino acid sequences, such as one or more nuclear localization signals (NLSs).

In some embodiments, a fusion protein of the disclosure comprises a means for localizing the engineered Type V CRISPR-Cas endonuclease to the nucleus via an NLS.

In some embodiments, a fusion protein of the disclosure comprises one or more NLS sequences positioned at the N-terminus and/or C-terminus of a B-GEn.2 protein sequence.

In some embodiments, a fusion protein of the disclosure comprises one or more NLS sequences only at the N-terminus of a B-GEn.2 protein sequence. In other embodiments, a fusion protein of the disclosure comprises one or more NLS sequences only at the C-terminus of a B-GEn.2 protein sequence.

In some embodiments, a fusion protein of the disclosure comprises multiple NLS sequences that are identical. In other embodiments, a fusion protein of the disclosure comprises multiple NLS sequences that are distinct.

Non-limiting examples of nuclear localization signals are listed in Table 3.

TABLE 3

| | | SEQ ID |
|---|---|---|
| NLS Sequence | NLS | NO |
| PKKKRKV | Wild-type SV40 large T | 7 |
| | protein | |
| KRPAATKKAGQAKKKK | Nucleoplasmin | 8 |
| PAAKRVKLD | c-myc | 9 |
| MSRRRKANPTKLSENAKKLAKEVEN | EGL-13 | 10 |
| KLKIKRPVK | TUS | 11 |
| (KR)-X(0,2)-(KR)-(KR)- | Basic PY-NLS | N/A |
| x(3,10)-(RHK)-X(1,5)-PY | | |
| PKKKRMV | SV40 large T protein, variant | 13 |
| | 1 | |
| PKKKRKWEDP | SV40 large T protein, variant | 14 |
| | 2 | |
| CGYGPKKKRKVGG | SV40 large T protein, variant | 15 |
| | 3 | |
| CGYGPKKKRKV | SV40 large T protein, variant | 16 |
| | 4 | |
| CYDDEATADSQHSTPPKKKRKWEDPK | SV40 large T protein long | 17 |
| DFESELLS | NLS | |
| CGGPKKKRKWG | SV40 large T protein, variant | 18 |
| | 5 | |
| PKKKIKW | SV40 large T protein, variant | 19 |
| | 6 | |
| KRTADGSEFESPKKKRKV | SV40 large T protein, variant | 20 |
| | 7 | |
| TKKAGQAKKK | Nucleoplasmin min-NLS | 21 |
| TKKAGQAKKKKLD | Nucleoplasmin NLS variant 1 | 22 |
| CGQAKKKKLD | Nucleoplasmin NLS variant 2 | 23 |
| RQRRNELKRSP | c-myc NLS2 | 24 |
| PKKARED | Polyoma large T protein | 25 |
| CGYGWSRKRPRPG | Polyoma virus large T protein | 26 |
| APTKRKGS | SV40 VP1 capsid polypeptide | 27 |
| APKRKSGVSKC | Polyoma virus major capsid | 28 |
| | protein VP1 (11 N-terminal | |
| | aa) | |
| PNKKKRK | SV40 VP2 capsid protein (39 | 29 |
| | KD) | |
| EEDGPQKKKRRL | Polyoma virus capsid protein | 30 |
| | VP2 | |
| GKKRSKA | Yeast histone H2B | 31 |
| KRPRP | Adenovirus E1a | 32 |
| CGGLSSKRPRP | Adenovirus type 2/5 E1a | 33 |
| LVRKKRKTE3SP | Xenopus N1, NLS1 | 34 |
| LKDKDAKKSKQE | Xenopus N1, NLS2 | 35 |
| GNKAKRQRST | V-Rel | 36 |
| PFLDRLRRDQK | NS1 protein of influenza A | 37 |
| | virus | |
| SVTKKRKLE | Human lamin A | 38 |
| SASKRRRLE | Xenopus lamin A | 39 |
| ACIDKRVKLD | Human c-myc | 40 |
| SALIKKKKKMAP | Murine c-abl | 41 |
| PPKKRMRRRIE | Adenovirus 5 DBP | 42 |
| YRKCLQAGMNLEARKTKKKIKGIQQATA | Rat glucocorticoid receptor | 43 |
| CGYGARKTKKKIK | Human glucocorticoid | 44 |
| | receptor | |
| RKCLQAGMNLEARKTKK | Human glucocorticoid | 45 |
| | receptor NLS variant | |
| RKFKKFNK | Rabbit progesterone receptor | 46 |
| CGYGIRKDRRGGR | Human estrogen receptor | 47 |
| CGYGARKLKKLGN | Human androgen receptor | 48 |
| GKRKNKPK | Chicken Ets1 core NLS | 49 |
| PLLKKIKQ | c-myb | 50 |
| PPQKKIKS | N-myc | 51 |
| PQPKKKP | p53 | 52 |
| PQPKKKPL | p53 NLS variant | 53 |
| SKRWAKRKL | c-erb-A | 54 |
| MTGSKTRKHRGSGA | Yeast ribosomal protein L29 | 55 |
| | NLS | |
| MTGSKHRKHPGSGA | Yeast ribosomal protein L29 | 56 |
| | NLS, variant 1 | |
| RHRKHP | Yeast ribosomal protein L29 | 57 |
| | NLS, variant 2 | |
| KRRKHP | Yeast ribosomal protein L29 | 58 |
| | NLS, variant 3 | |
| KYRKHP | Yeast ribosomal protein L29 | 59 |
| | NLS, variant 4 | |
| KHRRHP | Yeast ribosomal protein L29 | 60 |
| | NLS, variant 5 | |
| KHKKHP | Yeast ribosomal protein L29 | 61 |
| | NLS, variant 6 | |
| RHLKHP | Yeast ribosomal protein L29 | 62 |
| | NLS, variant 7 | |
| KHRKYP | Yeast ribosomal protein L29 | 63 |
| | NLS, variant 8 | |
| KHRQHP | Yeast ribosomal protein L29 | 64 |
| | NLS, variant 9 | |
| LVRKKRKTE3SP | Xenopus N1 NLS1 | 65 |
| LKDKDAKKSKQE | Xenopus N1 NLS2 | 66 |
| ASKSRKRKL | Viral Jun | 67 |
| GGLCSARLHRHALLAT | Human T-cell leukemia virus | 68 |
| | Tax trans-activator protein | |
| DTREKKKFLKRRLLRLDE | Mouse nuclear MX1 protein | 69 |
| | (72 kD) | |
| REKKKFLKRR | Mouse nuclear MX1 protein | 70 |
| | NLS variant | |
| CGYGDRNKKKKE | Human retinoic acid receptor | 71 |
| RKRQRALMLRQAR | Human XPAC | 72 |
| EYLSRKGKLEL | T-DNA-linked VirD2 | 73 |
| | endonuclease | |
| KKSKKKRC | Putative core NLS of yeast | 74 |
| | TRM1 | |
| QPQRYGGGRGRRW | Gag protein of human foamy | 75 |
| | retrovirus | |
| NKKKRKLSRGSSQKTKGTSASAKARH | SV40 Vp3 structural protein | 76 |
| KRRNRSSRS | | |
| RVTIRTWRWRRPPKGKHRK | Simian sarcoma virus v-sis | 77 |
| | gene product | |
| KRKIEEPEPEPKKAK | Putative bipartite NLS of | 78 |
| | Xenopus protein | |
| | factor Xnf7 | |
| KKYENVVIKRSPRKRGRPRKD | Yeast SWI5 gene product | 79 |
| GRKRAFHGDDPFGEGPPDKKGD | Herpes simplex virus ICP8 | 80 |
| | protein | |
| KRPREDDDGEPSERKRARDDR | Bipartite NLS of VirD2 | 81 |
| | endonuclease | |
| RMRIZFKNKGKDTAELRRRRVEVSVEL | IBB domain from importin- | 82 |
| RKAKKDEQILKRRNV | alpha | |
| NQSSNFGPMKGGNFGGRSSGPYGGG | hRNPAI M9 NLS | 83 |
| GQYFAKPRNQGGY | | |
| KRKGDEVDGVDEVAKKKSKK | Human poly(ADP-ribose) | 84 |
| | polymerase | |
| RKLKKKIKKL | Hepatitis virus delta antigen | 85 |
| PKQKKRK | influenza virus NLS | 86 |
| SALIKKKKKMAP | mouse c-abl IV | 87 |
| VSRKRPRP | myoma T protein NLS1 | 88 |
| PPKKARED | myoma T protein NLS2 | 89 |
| GPAAKRVKLD | myc proto-oncogene protein | 90 |
| | [Homo sapiens] | |
| KKRRIKQD | heat shock factor protein | 91 |
| | HSF8 [Solanum | |
| | lycopersicum] | |
| PKKKRKVEDPKKKRKVD | 2x SV40, LrgT | 92 |
| PKKKRKVDPKKKRKVDPKKKRKV | 3x SV40, LrgT | 93 |
| KKGKKKGK | monopartite NLS consensus | 94 |
| | cluster 1 (Dissertation | |
| | Tatyana Goldberg 2016) | |
| PKRRRGVVL | monopartite NLS consensus | 95 |
| | cluster 2 (Dissertation | |
| | Tatyana Goldberg 2016) | |
| EQLFKRRNV | monopartite NLS consensus | 96 |
| | cluster 3 (Dissertation | |
| | Tatyana Goldberg 2016) | |
| KRRRR | monopartite NLS consensus | 97 |
| | cluster 4 (Dissertation | |
| | Tatyana Goldberg 2016) | |
| KKRRR | monopartite NLS consensus | 98 |
| | cluster 5 (Dissertation | |
| | Tatyana Goldberg 2016) | |
| EGAPPAKRPR | monopartite NLS consensus | 99 |
| | cluster 6 (Dissertation | |
| | Tatyana Goldberg 2016) | |
| MLRRRRRKRAR | monopartite NLS consensus | 100 |
| | cluster 7 (Dissertation | |
| | Tatyana Goldberg 2016) | |
| RRKRR | monopartite NLS consensus | 101 |
| | cluster 8 (Dissertation | |
| | Tatyana Goldberg 2016) | |
| RKRK | monopartite NLS consensus | 102 |
| | cluster 9 (Dissertation | |
| | Tatyana Goldberg 2016) | |
| FKAVLEDILGEL | monopartite NLS minor | 103 |
| | cluster (Dissertation Tatyana | |
| | Goldberg 2016) | |
| KNRRL | NLSdb, protein origin P10152 | 104 |
| RKRHW | NLSdb, protein origin Q09353 | 105 |
| RRKKRR | NLSdb, protein origin | 106 |
| | QOVD86, Q58DS6, Q5R6G2, | |
| | Q9ERI5, Q6AYK2, Q6NYC1 | |
| RRKRSR | NLSdb, protein origin | 107 |
| | Q99PU7, D3ZHS6, Q92560, | |
| | A2VDM8 | |
| KRGRKP | NLSdb, protein origin | 108 |
| | Q14781, P30658 | |
| KKRKLE | NLSdb, protein origin | 109 |
| | P02545, P48678, P48679, | |
| | Q3ZD69 | |
| PKKKSRK | NLSdb, protein origin | 110 |
| | O35914, Q01954 | |
| PKRGRGR | NLSdb, protein origin | 111 |
| | Q9FYS5, Q43386 | |
| KEKRKKR | NLSdb, protein origin | 112 |
| | E5RQA1 | |
| KKKKRKR | NLSdb, protein origin | 113 |
| | Q9Z1J1, Q9HCS4, Q924A0 | |
| RRGDGRRR | NLSdb, protein origin | 114 |
| | Q80WE1, Q5R9B4, Q06787, | |
| | P35922 | |
| LSPSLSPL | NLSdb, protein origin | 115 |
| | Q9Y261, P32182, P35583 | |
| VNFSEFSK | NLSdb, protein origin | 116 |
| | P07156 | |
| IVINILSE | NLSdb, protein origin | 117 |
| | Q96EB6 | |
| PPAKRKCIF | NLSdb, protein origin | 118 |
| | Q6AZ28, 075928, Q8C5D8 | |
| QRPGPYDRP | NLSdb, SeqNLS prediction | 119 |
| KRKRGRPRK | NLSdb, protein origin | 120 |
| | Q8L7L5, A1L4X7, O80834, | |
| | Q8LPN5 | |
| KIKELYRRR | NLSdb, protein origin | 121 |
| | 088907, 075925 | |
| MVQLRPRASR | NLSdb, SeqNLS prediction | 122 |
| KKRREKQRRR | NLSdb, protein origin | 123 |
| | Q5VK71 | |
| EGAPPAKRAR | NLSdb, protein origin | 124 |
| | POC6L6, P25880, Q81835, | |
| | P29833, P25882, P06934, | |
| | POC6L3, P29997, POC6M5, | |
| | POC6M9, P29996, POC6M1, | |
| | POC6L8, POC6M2, POC6L7, | |
| | P25881 | |
| PKKGDKYDKTD | NLSdb, protein origin | 125 |
| | Q45FA5 | |
| KKKKSKDKKRK | NLSdb, protein origin | 126 |
| | P97376, Q14331 | |
| AHRAKKMSKTHA | NLSdb, protein origin P21827 | 127 |
| KKGPSVQKRKKT | NLSdb, protein origin | 128 |
| | Q6ZN17 | |
| KGVKRKADTTTP | NLSdb, protein origin | 129 |
| | Q4R8Y1 | |
| KGVKRRADTTTP | NLSdb, protein origin | 130 |
| | Q91Y44, D4A7T3 | |
| KKPKWDDFKKKKK | NLSdb, protein origin | 131 |
| | Q15397, Q8BKS9, Q562C7 | |
| KRRRRRRREKRKR | NLSdb, protein origin | 132 |
| | Q96GM8 | |
| DVRKRVQDLEQKM | NLSdb, protein origin | 133 |
| | P61635, Q6DV79, Q19S50, | |
| | P52631 | |
| KKGKDEWFSRGKK | NLSdb, protein origin 060716 | 134 |
| KKGKDEWFSRGKKP | NLSdb, protein origin P30999 | 135 |
| ASPEYVNLPINGNG | NLSdb, SeqNLS prediction | 136 |
| YLRPVKKPKIRRKK | NLSdb, protein origin | 137 |
| | Q7Z7C8, Q5ZMS1, Q9EQH4, | |
| | A7MAZ4 | |
| KRKGKLKNKGSKRKK | NLSdb, protein origin O15381 | 138 |
| RRRGKNKVAAQNCRK | NLSdb, SeqNLS prediction | 139 |
| DKAKRVSRNKSEKKRR | NLSdb, protein origin | 140 |
| | O15516, Q5RAK8, Q91YB2, | |
| | Q91YB0, Q8QGQ6, O08785, | |
| | Q9WVS9, Q6YGZ4 | |
| EEQLRRRKNSRLNNTG | NLSdb, protein origin | 141 |
| | G5EFF5 | |
| HKKKHPDASVNFSEFSK | NLSdb, protein origin | 142 |
| | P10103, Q4R844, P12682, | |
| | BOCM99, A9RA84, Q6YKA4, | |
| | P09429, P63159, Q08IE6, | |
| | P63158, Q9YH06, B1MTB0 | |
| KKTGKNRKLKSKRVKTR | NLSdb, protein origin | 143 |
| | Q9Z301, 054943, Q8K3T2 | |
| KRSCRRRLAGHNERRRK | NLSdb, protein origin | 144 |
| | Q38740, Q38741, Q700W2, | |
| | Q9S7A9, Q6Z461, P93015, | |
| | Q94JW8, Q9S758 | |
| KRQRRKQSNRESARRSR | NLSdb, protein origin | 145 |
| | Q501B2 | |
| RGKGGKGLGKGGAKRHRK | NLSdb, SeqNLS prediction | 146 |
| RRRGFERFGPDNMGRKRK | NLSdb, protein origin | 147 |
| | Q63014, Q9DBRO | |
| RRHQQGQGDDSSHKKERK | NLSdb, protein origin Q0IJ08, | 148 |
| | Q2TAE3, Q63470, Q13627, | |
| | Q61214 | |
| KKKTGVIAPKRFVQRLKK | NLSdb, protein origin | 149 |
| | Q8LAM0, O24454 | |
| KRAMKDDSHGNSTSPKRRK | NLSdb, protein origin | 150 |
| | QUE671 | |
| KVNFLDMSLDDIIIYKELE | NLSdb, protein origin | 151 |
| | Q9P127 | |
| KKYENVVIKRSPRKRGRPRK | NLSdb, SeqNLS prediction | 152 |
| KRGNSSIGPNDLSKRKQRKK | NLSdb, SeqNLS prediction | 153 |
| KRASEDTTSGSPPKKSSAGPKR | NLSdb, protein origin | 154 |
| | Q9BZZ5, Q5R644 | |
| KRIHSVSLSQSQIDPSKKVKRAK | NLSdb, SeqNLS prediction | 155 |
| EVLKVIRTGKRKKKAWKRMVTKVC | NLSdb, SeqNLS prediction | 156 |
| IINGRKLKLKKSRRRSSQTSNNSFTSRRS | NLSdb, SeqNLS prediction | 157 |
| AHFKISGEKRPSTDPGKKAKNPKKKKKKDP | NLSdb, protein origin Q76IQ7 | 158 |

### 6.5. The Linker Sequence

The disclosure provides engineered Type V CRISPR-Cas endonucleases, which are in the form of fusion proteins comprising a B-GEn nuclease sequence fused with one or more NLS sequences, optionally via a peptide linker.

In some embodiments, a B-GEn nuclease sequence is connected to an NLS sequence on its N- and/or C-terminus via a peptide linker. In other embodiments, a B-GEn nuclease sequence is connected to one or more NLS sequences with a peptide linker connecting a pair of individual polypeptide sequences, such as between two NLS sequences or between an NLS sequence and a B-GEn nuclease sequence.

In some embodiments, a B-GEn polypeptide of the disclosure comprises multiple NLS sequences that are connected by identical linkers. In other embodiments, a B-GEn polypeptide of the disclosure comprises multiple NLS sequences that are connected by distinct linkers.

Peptide linkers suitable for use in the B-GEn polypeptides of the disclosure include those disclosed in Chen et al., 2013, Adv Drug Deliv Rev. 65(10):1357-1369. Nonlimiting examples of such linkers are reproduced below in Table 4.

TABLE 4

| | | SEQ ID |
|---|---|---|
| Linker Sequence | Linker Type | NO |
| (GGGGS)₃ | Flexible | 159 |
| GGG(EAAAK)₃ | Mixed | 160 |
| A(EAAAK)₄ALEA | Rigid | 161 |
| (EAAAK)₄A | | |
| GGGGSLVPRGSGG | Flexible | 162 |
| GGS | | |
| GAAPAAAPAKQEA | Proline rich, | 163 |
| AAPAPAAKAEAPA | rigid, | |
| AAPAAKA | interdomain | |
| TRHRQPRGWE | Cleavable | 164 |

### 6.6. B-GEn Type V CRISPR-Cas Systems

The disclosure provides engineered B-GEn Type V CRISPR-Cas endonuclease systems incorporating the engineered B-GEn polypeptides of the disclosure or nucleic acids encoding them.

In some embodiments, an engineered B-GEn Type V CRISPR-Cas endonuclease system comprises the following components:
(a) an engineered B-GEn polypeptide, e.g., as described in Section 6.2, or a nucleic acid encoding such engineered B-GEn polypeptide, e.g., as described in Section 6.9,
(b) a heterologous guide RNA (gRNA), e.g., as described in Section 6.7, or a nucleic acid (e.g., a vector as described in Section 6.10) that allows the generation of such gRNA in situ, wherein the gRNA comprise(s):
   i. an engineered DNA targeting segment composed of RNA and capable of hybridizing to a target sequence in a nucleic acid locus,
   ii. a tracr mate sequence composed of RNA, and
   iii. a tracr RNA sequence composed of RNA, wherein the tracr mate sequence hybridizes to the tracr sequence, and wherein (i), (ii), and (iii) are arranged in a 5′ to 3′ orientation. The gRNA can be a single guide RNA (sgRNA). Within a sgRNA, a tracr mate sequence and a tracr sequence are generally connected by a suitable loop sequence and form a stem-loop structure.

In particular embodiments, an engineered B-GEn Type V CRISPR-Cas endonuclease system comprises the following components:
(a) an engineered B-GEn polypeptide, e.g., as described in Section 6.2,
(b) a heterologous guide RNA (gRNA), e.g., as described in Section 6.7, which comprise(s):
   i. an engineered DNA targeting segment composed of RNA and capable of hybridizing to a target sequence in a nucleic acid locus,
   ii. a tracr mate sequence composed of RNA, and
   iii. a tracr RNA sequence composed of RNA,
wherein the tracr mate sequence hybridizes to the tracr sequence, and wherein (i), (ii), and (iii) are arranged in a 5′ to 3′ orientation. The gRNA can be a single guide RNA (sgRNA). Within a sgRNA, a tracr mate sequence and a tracr sequence are generally connected by a suitable loop sequence and form a stem-loop structure.

In some embodiments, such engineered B-GEn Type V CRISPR-Cas endonucleases are delivered to target cells in a composition known as a ribonucleoprotein (RNP) complex described in Section 6.8.

In particular embodiments, engineered B-GEn Type V CRISPR-Cas endonuclease systems comprise the following components:
(a) a nucleic acid, e.g., as described in Section 6.9, encoding an engineered B-GEn polypeptide, e.g., as described in Section 6.2, or
(b) a nucleic acid (e.g., a vector as described in Section 6.10) that allows the generation of a heterologous guide RNA (gRNA), e.g., as described in Section 6.7, wherein the gRNA comprise(s):
   i. an engineered DNA targeting segment composed of RNA and capable of hybridizing to a target sequence in a nucleic acid locus,
   ii. a tracr mate sequence composed of RNA, and
   iii. a tracr RNA sequence composed of RNA,
wherein the tracr mate sequence hybridizes to the tracr sequence, and wherein (i), (ii), and (iii) are arranged in a 5′ to 3′ orientation. The gRNA can be a single guide RNA (sgRNA). Within a sgRNA, a tracr mate sequence and a tracr sequence are generally connected by a suitable loop sequence and form a stem-loop structure.

Any reference to “RNA” or “guide RNA” encompasses RNA molecules comprising non-natural as well as natural nucleobases, for example one or more of the nucleic acid modifications described in Section 6.9.3.

In some embodiments, the nucleic acid(s) encoding the engineered B-GEn polypeptide and/or the sgRNAs contain a suitable promoter for the expression in a cellular or in vitro environment.

In some embodiments, the nucleic acid(s) encoding the engineered B-GEn polypeptide and/or the sgRNAs is in the form of a viral vector, for example as described in Section 6.10.3.

### 6.7. Guide RNAs (gRNAs) and Single Guide RNAs (sgRNAs)

The systems, compositions, and methods described herein in some embodiments employ a genome-targeting nucleic acid that can direct the activities of an engineered B-GEn polypeptide to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that can hybridize to a target nucleic acid sequence of interest and a CRISPR repeat sequence (such a CRISPR repeat sequence is also referred to as a “tracr mate sequence”). In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-specific polypeptide such that the guide RNA and site-direct polypeptide form a complex. The genome-targeting nucleic acid provides target specificity to the complex by virtue of its association with the site-specific polypeptide. The genome-targeting nucleic acid thus directs the activity of the site-specific polypeptide.

In some embodiments, the genome-targeting nucleic acid is a double-molecule guide RNA. In some embodiments, the genome-targeting nucleic acid is a single-molecule guide RNA or single guide RNA (sgRNA). A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins.

A single-molecule guide RNA (sgRNA) in a Type V system has, in the 5′ to 3′ direction, a minimum CRISPR repeat sequence and a spacer sequence. Or alternatively, a single-molecule guide RNA (sgRNA) in a Type V system has, in the 5′ to 3′ direction, optional tracr extension sequence, a tracr RNA sequence, a single molecule guide linker, a minimum CRISPR repeat sequence, a spacer sequence, and an optional spacer extension sequence.

Alternatively, a single-molecule guide RNA (sgRNA) in a Type V system has, in the 5′ to 3′ direction, an optional extension sequence, a minimum CRISPR repeat sequence, a spacer sequence, and an optional spacer extension sequence.

In yet another embodiment, an sgRNA in a Type V system comprises, in the 5′ to 3′ direction, an optional extension sequence, an artificial nuclease binding RNA sequence and a spacer sequence, and an optional spacer extension sequence.

Particularly useful sgRNAs for the B-GEn.2 CRISPR Cas nucleases according to the disclosure, and potentially for other type V CRISPR Cas nucleases, are disclosed in Table 5.

TABLE 5

| | | SEQ |
|---|---|---|
| | | ID |
| sgRNA | Sequence | NO |
| B-GEn.2- | guuuagcuau aggcuaauaa gauaguugug | 165 |
| sgRNA_v4 | ucaagugcuu cggagaccua acacgucauc | |
| | cagucacaac ggcuaucuaa aaauagaugg | |
| | uuagcac | |
| B-GEn.2- | uuagcuauag gcuaauaaga uaguuguguc | 166 |
| sgRNA_v4.2 | aagugcuucg gagaccuaac acgucaucca | |
| | gucacaacgg cuaaaaaugg uuagcac | |
| B-GEn.2- | uuagcuauag gcuaauaaga uaguuguguc | 167 |
| sgRNA_v4.3 | aagugcuucg gagaccuaac acgucaucca | |
| | gucacaacgg cuaaaaauag ccagcac | |
| B-GEn.2- | uuagcuauag guaauaaga uaguuguguc | 168 |
| sgRNA_v4.4 | aagugcuucg gagaccuaac acgucuccag | |
| | ucacaacggc uaaaaauagc cagcac | |
| B-GEn.2- | uuagcuauag gcuaauaaga uaguugugac | 169 |
| sgRNA_v4.5 | aagugcuucg gagaccuaac acgucuccag | |
| | ucacaacggc uaaaaauagc cagcac | |

Exemplary genome-targeting nucleic acids are described, for example, in WO2018002719. In general, a CRISPR repeat sequence includes any sequence that has sufficient complementarity with a tracr sequence to promote one or more of: (1) excision of a DNA targeting segment flanked by CRISPR repeat sequences in a cell containing the corresponding tracr sequence; and (2) formation of a CRISPR complex at a target sequence, wherein the CRISPR complex includes the CRISPR repeat sequence hybridized to the tracr sequence. In general, degree of complementarity is with reference to the optimal alignment of the CRISPR repeat sequence and tracr sequence, along the length of the shorter of the two sequences. Optimal alignment may be determined by any suitable alignment algorithm and may further account for secondary structures, such as self-complementarity within either the tracr sequence or CRISPR repeat sequence. In some embodiments, the degree of complementarity between the tracr sequence and CRISPR repeat sequence along the 30 nucleotides length of the shorter of the two when optimally aligned is about or more than 25%, 30%, 40%, 50%, 60%, 70%, 80%, 90%, 95%, 97.5%, 99%, or higher. In some embodiments, the tracr sequence is about or more than 5, 6, 7, 8, 9, 10, 11, 12, 13, 14, 15, 16, 17, 18, 19, 20, 25, 30, 40, 50, or more nucleotides in length. In some embodiments, the tracr sequence and CRISPR repeat sequence are contained within a single transcript, such that hybridization between the two produces a transcript having a secondary structure, such as a hairpin. In some embodiments, the transcript or transcribed nucleic acid sequence has at least two or more hairpins.

Suitable tracr sequences for use with B-GEn.2 or B-GEn.1 in CRISPR Cas systems are listed in Table 6. Alternatively, variants of these sequences could be employed. Variants could include either parts or truncated versions of such sequences and/or sequences with base modifications in one or more places of these sequences. The respective RNA sequences are disclosed in SEQ ID NOs: 170 and 171, respectively.

TABLE 6

| | SEQ ID | |
|---|---|---|
| Name | NO: | RNA sequence |
| tracr_B- | 170 | UAAUAAAUAAUAUGAGUUACAG |
| GEn.1 | | GCUCAUGUCAUUCCGUGCGUGU |
| | | GCUGCGAGGCUCUUACAACGUC |
| | | UCAACCACGGGAUCACGUUAUU |
| | | UCCACUAAGUGAUUGGAC |
| tracr_B- | 171 | CUGGAUAUAAUAAAGAUGUUUA |
| GEn.2 | | GCUAUAGGCUAAUAAGAUAGUU |
| | | GUGUCAAGUGCUUCGGAGACCU |
| | | AACACGUCAUCCAGUCACAACG |
| | | GCUAUCUAUAUUUCCACUAAC |

The spacer of a guide RNA includes a nucleotide sequence that is complementary to a sequence in a target DNA. In other words, the spacer of a guide RNA interacts with a target DNA in a sequence-specific manner via hybridization (e.g., base pairing). As such, the nucleotide sequence of the spacer may vary and determines the location within the target DNA that the guide RNA and the target DNA will interact. The DNA-targeting segment of a guide RNA can be modified (e.g., by genetic engineering) to hybridize to any desired sequence within a target DNA.

In some embodiments, the spacer has a length of from 10 nucleotides to 30 nucleotides. In some embodiments, the spacer has a length of from 13 nucleotides to 25 nucleotides. In some embodiments, the spacer has a length of from 15 nucleotides to 23 nucleotides. In some embodiments, the spacer has a length of from 18 nucleotides to 22 nucleotides, e.g., from 20 to 22 nucleotides.

In some embodiments, the percent complementarity between the DNA-targeting sequence of the spacer and the protospacer of the target DNA is at least 60% (e.g., at least 65%, at least 70%, at least 75%, at least 80%, at least 85%, at least 90%, at least 95%, at least 96%, at least 97%, at least 98%, at least 99%, or 100%) over the 20-22 nucleotides.

In some embodiments, the protospacer is directly adjacent to a suitable PAM sequence on its 3′ end or such PAM sequence is part of the DNA targeting sequence in its 3′ portion.

Suitable PAM sequences are listed in Table 7, wherein the engineered DNA targeting segment is, on its 3′ end, directly adjacent to the PAM sequence on the targeted DNA segment, or such PAM sequence is part of the targeted DNA sequence in its 5′ portion.

TABLE 7

| B-GEn | PAM Sequence |
|---|---|
| B-GEn.1 | “DTTN” with “D” representing “A” or “T” or “G”; |
| | preferably “ATT” |
| B-GEn.1.2 | “DTTN” with “D” representing “A” or “T” or “G”; |
| | preferably “ATT” |
| B-GEn.2 | “DTTN” with “D” representing “A” or “T” or “G”; |
| | preferably “ATT” |

Modifications of guide RNAs can be used to enhance the formation or stability of the CRISPR-Cas genome editing complex comprising guide RNAs and a Cas endonuclease such as B-GEn.1 or B-GEn.2. Modifications of guide RNAs can also or alternatively be used to enhance the initiation, stability or kinetics of interactions between the genome editing complex with the target sequence in the genome, which can be used for example to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas endonuclease such as a B-GEn.1, or B-GEn.1.2, or B-GEn.2 is introduced into the cell to be edited via an RNA that needs to be translated in order to generate B-GEn.1, or B-GEn.1.2, or B-GEn.2 endonuclease, since increasing the half-life of guide RNAs introduced at the same time as the RNA encoding the endonuclease can be used to increase the time that the guide RNAs and the encoded Cas endonuclease co-exist in the cell.

### 6.7.1. Additional Sequences

In some embodiments, a guide RNA comprises at least one additional segment at either the 5′ or 3′ end. For example, a suitable additional segment can comprise a 5′ cap (e.g., a 7-methylguanylate cap (m7G)); a 3′ polyadenylated tail (e.g., a 3′ poly(A) tail); a riboswitch sequence (e.g., to allow for regulated stability and/or regulated accessibility by proteins and protein complexes); a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability; and combinations thereof.

### 6.7.1.1. Stability Control Sequences

A stability control sequence influences the stability of an RNA (e.g., a guide RNA). A non-limiting example of a suitable stability control sequence is a transcriptional terminator segment (e.g., a transcription termination sequence). A transcriptional terminator segment of a guide RNA can have a total length of from 10 nucleotides to 100 nucleotides, e.g., from 10 nucleotides (nt) to 20 nt, from 20 nt to 30 nt, from 30 nt to 40 nt, from 40 nt to 50 nt, from 50 nt to 60 nt, from 60 nt to 70 nt, from 70 nt to 80 nt, from 80 nt to 90 nt, or from 90 nt to 100 nt. For example, the transcriptional terminator segment can have a length of from 15 nucleotides (nt) to 80 nt, from 15 nt to 50 nt, from 15 nt to 40 nt, from 15 nt to 30 nt or from 15 nt to 25 nt.

In some embodiments, the transcription termination sequence is one that is functional in a eukaryotic cell. In some embodiments, the transcription termination sequence is one that is functional in a prokaryotic cell.

Nucleotide sequences that can be included in a stability control sequence (e.g., transcriptional termination segment, or in any segment of the guide RNA to provide for increased stability) include, for example, a Rho-independent trp termination site.

### 6.8. Ribonucleoprotein (RNP) Complexes

In some embodiments, the engineered B-GEn Type V CRISPR-Cas endonucleases are delivered in a composition known as a ribonucleoprotein or RNP complex. An RNP complex is assembled by combining a Cas endonuclease, such as an engineered B-GEn endonuclease, with a ribonucleic acid, e.g., a guide RNA (gRNA).

In some embodiments, the ribonucleoprotein complex comprises an engineered B-GEn endonuclease, e.g., as described in Section 6.3, complexed with a suitable ribonucleic acid. In some embodiments, the ribonucleic acid is a gRNA or an sgRNA, which are described further in Section 6.7. In some embodiments, the RNP complex comprises an engineered B-GEn polypeptide and an sgRNA listed in Table 5 or another suitable sgRNA.

One of the most common techniques for delivery of RNPs is electroporation, which generates pores in the cell membrane, allowing for entry of the RNP into the cytoplasm. Further, electroporation can be combined with cell-type specific reagents in a technique known as nucleofection, which forms pores in the nuclear membrane, allowing for entry of a DNA template. In some embodiments, an engineered B-GEn Type V CRISPR-Cas endonuclease in an RNP complex is delivered into target cells via nucleofection.

### 6.9. Nucleic Acids

The disclosure provides nucleic acids (e.g., DNA or RNA) encoding B-GEn Type V CRISPR-Cas proteins (e.g., an engineered B-GEn polypeptide), nucleic acids encoding gRNAs or sgRNAs of the disclosure, nucleic acids encoding both engineered B-GEn polypeptides and gRNAs or sgRNAs, and pluralities of nucleic acids, for example comprising a nucleic acid encoding an engineered B-GEn polypeptide and a gRNA or an sgRNA.

Nucleic acids encoding an engineered B-GEn polypeptide can be codon optimized, e.g., where at least one non-common codon or less-common codon has been replaced by a codon that is common in a host cell or target cell. For example, a codon optimized nucleic acid can direct the synthesis of an optimized messenger mRNA, e.g., optimized for expression in a mammalian expression system.

### 6.9.1. B-GEn Coding Sequences

In some embodiments, a nucleic acid described herein comprises one or more modifications which can be used, for example, to enhance activity, stability or specificity, alter delivery, reduce innate immune responses in host cells, further reduce the protein size, or for other enhancements, as further described herein and known in the art. In some embodiments, such modifications will result in an engineered B-GEn polypeptide whose nuclease sequence component has at least 75%, at least 80%, at least 85%, at least 90%, at least 95%, at least 99%, or 100% amino acid sequence identity to the sequence of SEQ ID NOs: 4, 5, or 6.

### 6.9.2. Codon Optimization

In certain embodiments, modified nucleic acids are used in a CRISPR-B-GEn.1, or B-GEn.1.2, or B-GEn.2 system described herein, in which the guide RNAs and/or a DNA or an RNA comprising a nucleic acid sequence encoding an engineered B-GEn polypeptide can be modified, as described below. Such modified nucleic acids can be used in the CRISPR-B-GEn.1, or B-GEn.1.2, or B-GEn.2 system to edit any one or more genomic loci. In some embodiments, such modifications in the nucleic acids of the disclosure are achieved via codon-optimization, e.g., codon-optimized based on specific host cells in which the encoded polypeptide is expressed. It will be appreciated by the skilled artisan that any nucleotide sequence and/or recombinant nucleic acid of the present disclosure can be codon optimized for expression in any species of interest. Codon optimization is well known in the art and involves modification of a nucleotide sequence for codon usage bias using species specific codon usage tables. The codon usage tables are generated based on a sequence analysis of the most highly expressed genes for the species of interest. In a non-limiting example, when the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native nucleic acid sequences.

In some embodiments, an engineered B-GEn polypeptide described herein is expressed from a codon-optimized nucleic acid sequence. For example, if the intended host cell or target cell were a human cell, a human codon-optimized nucleic acid sequence encoding an engineered B-GEn polypeptide comprising the amino acid sequence of B-GEn.1, or B-GEn.1.2, or B-GEn.2 (or a B-GEn.1, or B-GEn.1.2, or B-GEn.2 variant, e.g., enzymatically inactive variant) would be suitable. As another non-limiting example, if the intended host cell or target cell were a mouse cell, then a mouse codon-optimized nucleic acid sequence an engineered B-GEn polypeptide comprising the amino acid sequence of B-GEn.1, or B-GEn.1.2, or B-GEn.2 (or a B-GEn.1, or B-GEn.1.2, or B-GEn.2 variant, e.g., enzymatically inactive variant) would be suitable.

Strategies and methodologies for codon optimization are known in the art and have been described for various systems including, but not limited to yeast (Outchkourov et al., Protein Expr Purif, 24(1):18-24 (2002)) and E. coli (Feng et al., Biochemistry, 39(50):15399-15409 (2000)). In some embodiments, the codon optimization is performed by using GeneGPS® Expression Optimization Technology (ATUM) and using the manufacturer's recommended expression optimization algorithms. In some embodiments, the nucleic acids of the disclosure are codon-optimized for increased expression in a human cell. In some embodiments, the nucleic acids of the disclosure are codon-optimized for increased expression in an E. coli cell. In some embodiments, the nucleic acids of the disclosure are codon-optimized for increased expression in an insect cell. In some embodiments, the nucleic acids of the disclosure are codon-optimized for increased expression in a Sf9 insect cell. In some embodiments, the expression optimization algorithms used in codon optimization procedure are defined to avoid putative poly-A signals (e.g., AATAAA and ATTAAA) as well as long (greater than 4) stretches of A's which can lead to polymerase slippage.

As is well understood in the art, codon optimization of a nucleotide sequence results in a nucleotide sequence having less than 100% identity (e.g., less than 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) to the native nucleotide sequence but which still encodes a polypeptide having the same function as that encoded by the original, native nucleotide sequence. Thus, in representative embodiments of the disclosure, the nucleotide sequence and/or recombinant nucleic acid of the disclosure can be codon optimized for expression in the particular species of interest.

In some embodiments, a codon-optimized nucleic acid sequence has at least 90%, 92%, 93%, 94%, 95%, 96%, 97%, 98%, 99%, 99.2%, 99.5%, 99.8%, 99.9%, or 100% sequence identity to SEQ ID NO: 4. In some embodiments, the nucleic acids of the disclosure are codon-optimized for increased expression of the encoded engineered B-GEn polypeptide in a target cell or a host cell. In some embodiments, the nucleic acids of the disclosure are codon-optimized for increased expression in a human cell. Generally, the nucleic acids of the disclosure are codon-optimized for increased expression in any human cells. In some embodiments, the nucleic acids of the disclosure are codon-optimized for increased expression in an E. coli cell. In some embodiments, the nucleic acids of the disclosure are codon-optimized for increased expression in an insect cell. Generally, the nucleic acids of the disclosure are codon-optimized for increased expression in any insect cells. In some embodiments, the nucleic acids of the disclosure are codon-optimized for increased expression in a Sf9 insect cell expression system.

Polyadenylation signals can also be chosen to optimize expression in the intended host.

### 6.9.3. Nucleic Acid Modifications

In some embodiments, a nucleic acid (e.g., a guide RNA, a nucleic acid comprising a nucleotide sequence encoding a guide RNA; a nucleic acid encoding a site-specific modifying enzyme such as an engineered B-GEn polypeptide of the disclosure; etc.) includes a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (e.g., a 3′ poly(A) tail); a riboswitch sequence (e.g., to allow for regulated stability and/or regulated accessibility by proteins and/or protein complexes); a stability control sequence; a sequence that forms a dsRNA duplex (e.g., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

In some embodiments, a guide RNA includes an additional segment at either the 5′ or 3′ end that provides for any of the features described above. For example, a suitable third segment can include a 5′ cap (e.g., a 7-methylguanylate cap (m7G)); a 3′ polyadenylated tail (e.g., a 3′ poly(A) tail); a riboswitch sequence (e.g., to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA. including transcriptional activators, transcriptional repressors, DNA methyltransferases, DNA demethylases, histone acetyltransferases, histone deacetylases, and the like); and combinations thereof.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an engineered B-GEn polypeptide that are introduced into a cell, including, without limitation, modifications that enhance the stability of the RNA (such as by decreasing its degradation by RNases present in the cell), modifications that enhance translation of the resulting product (e.g., the endonuclease), and/or modifications that decrease the likelihood or degree to which the RNAs introduced into cells elicit innate immune responses. Combinations of modifications, such as the foregoing and others, can likewise be used. In the case of an engineered B-GEn polypeptide, for example, one or more types of modifications can be made to guide RNAs (including those exemplified above), and/or one or more types of modifications can be made to RNAs encoding an engineered B-GEn polypeptide (including those exemplified above).

By way of illustration, guide RNAs used in the CRISPR-B-GEn system or other smaller RNAs can be readily synthesized by chemical means, enabling a number of modifications to be readily incorporated, as illustrated below and described in the art. While chemical synthetic procedures are continually expanding, purifications of such RNAs by procedures such as high-performance liquid chromatography (HPLC, which avoids the use of gels such as PAGE) tends to become more challenging as nucleic acid lengths increase significantly beyond a hundred or so nucleotides. One approach used for generating chemically modified RNAs of greater length is to produce two or more molecules that are ligated together. Much longer RNAs, such as those encoding a B-GEn.1, or B-GEn.1.2, or B-GEn.2 endonuclease, are more readily generated enzymatically. While fewer types of modifications are generally available for use in enzymatically produced RNAs, there are still modifications that can be used to, e.g., enhance stability, reduced the likelihood or degree of innate immune response, and/or enhance other attributes, as described further below and in the art; and new types of modifications are regularly being developed.

By way of illustration of various types of modifications, especially those used frequently with smaller chemically synthesized RNAs, modifications can include one or more nucleotides modified at the 2′ position of the sugar, in some embodiments a 2′-O-alkyl, 2′-O-alkyl-O-alkyl or 2′-fluoro-modified nucleotide. In some embodiments, RNA modifications include 2′-fluoro, 2′-amino and 2′ O-methyl modifications on the ribose of pyrimidines, basic residues or an inverted base at the 3′ end of the RNA. Such modifications are routinely incorporated into oligonucleotides and these oligonucleotides have been shown to have a higher Tm (e.g., higher target binding affinity) than; 2′-deoxy oligonucleotides against a given target.

A number of nucleotide and nucleoside modifications have been shown to make the oligonucleotide into which they are incorporated more resistant to nuclease digestion than the native oligonucleotide; these modified oligonucleotides survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH2-NH—O—CH2, CH, —N(CH3)-O—CH2 (known as a methylene(methylimino) or MMI backbone), CH2-O—N(CH3)-CH2, CH2-N(CH3)-N(CH3)-CH2 and O-N(CH3)-CH2-CH2 backbones; amide backbones (see De Mesmaeker et al., 1995, Ace. Chem. Res., 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177, 196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Braasch and Corey, Biochemistry, 41(14): 4503-4510 (2002); Genesis, Volume 30, Issue 3, (2001); Heasman, Dev. Biol., 243:209-214 (2002); Nasevicius et al., Nat. Genet., 26:216-220 (2000); Lacenra etc., Proc. Natl. Acad. Sci., 97: 9591-9596 (2000); and U.S. Pat. No. 5,034,506, issued Jul. 23, 1991. Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, 0, Sand CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH3, F, OCN, OCH3, OCH3 O(CH2)n CH3, O(CH2)n NH2 or O(CH2)n CH3 where n is from 1 to 10; C1 to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl: SOCH3; SO2CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some embodiments, a modification includes 2′-methoxyethoxy (2′-O—CH2CH2OCH3, also known as 2′-O-(2-methoxyethyl)) (Martin et al, Helv. Chim. Acta, 1995, 78, 486). Other modifications include 2′-methoxy (2′-O—CH3), 2′-propoxy (2′-OCH2 CH2CH3) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group. In some embodiments, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al., Science, 254: 1497-1500 (1991).

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A, DNA Replication, W. H. Freeman & Co., San Francisco, pp75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by 0.6-1.2 degrees C. (Sanghvi, Y. S., in Crooke, S. T. and Lebleu, B., eds., Antisense Research and Applications, CRC Press, Boca Raton, 1993, pp. 276-278) and are embodiments of base substitutions.

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other a-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Other useful nucleobases include those disclosed in U.S. Pat. No. 3,687,808, those disclosed in “The Concise Encyclopedia of Polymer Science And Engineering”, pages 858-859, Kroschwitz, J.I., ed. John Wiley & Sons, 1990, those disclosed by Englisch et al., Angewandte Chemie, International Edition, 1991, 30, page 613, and those disclosed in Sanghvi, Y. S., Chapter 15, Antisense Research and Applications, pages 289-302, Crooke, S. T. and Lebleu, B. ea., CRC Press, 1993. Certain of these nucleobases are particularly useful for increasing the binding affinity of the oligomeric compounds of the disclosure. These include 5-substituted pyrimidines, 6-azapyrimidines and N-2, N-6 and —O-6 substituted purines, including 2-aminopropyladenine, 5-propynyluracil and 5-propynylcytosine. 5-methylcytosine substitutions have been shown to increase nucleic acid duplex stability by 0.6-1.2 oc (Sanghvi, Y. S., Crooke, S. T. and Lebleu, B., eds, “Antisense Research and Applications”, CRC Press, Boca Raton, 1993, pp. 276-278) and are embodiments of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Patent Application Publication 20030158403.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, the guide RNAs and/or mRNA encoding an endonuclease such as B-GEn.1, or B-GEn.1.2, or B-GEn.2 of the disclosure are capped using any one of current capping methods such as mCAP, ARCA or enzymatic capping methods to create viable mRNA constructs that remain biologically active and avoid self/non-self intracellular responses. In some embodiments, the guide RNAs and/or mRNA encoding an endonuclease such as B-GEn.1, or B-GEn.1.2, or B-GEn.2 of the disclosure are capped by using a CleanCap™ (TriLink) co-transcriptional capping method.

In some embodiments, the guide RNAs and/or mRNA encoding an endonuclease of the disclosure includes one or more modifications selected from the group consisting of pseudouridine, N1-methylpseudouridine, and 5-methoxyuridine. In some embodiments, one or more N1-methylpseudouridines are incorporated into the guide RNAs and/or mRNA encoding an endonuclease of the disclosure in order to provide enhanced RNA stability and/or protein expression and reduced immunogenicity in animal cells, such as mammalian cell (e.g., human and mice). In some embodiments, the N1-methylpseudouridine modifications are incorporated in combination with one or more 5-methylcytidines.

In some embodiments, the guide RNAs and/or mRNA (or DNA) encoding an endonuclease such as B-GEn.1, or B-GEn.1.2, or B-GEn.2 are chemically linked to one or more moieties or conjugates that enhance the activity, cellular distribution, or cellular uptake of the oligonucleotide. Such moieties include but are not limited to, lipid moieties such as a cholesterol moiety (Letsinger et al., 1989, Proc. Natl. Acad. Sci. USA 86: 6553-6556); cholic acid (Manoharan et al., 1994, Bioorg. Med. Chem. Let. 4: 1053-1060); a thioether, e.g., hexyl-S-tritylthiol (Manoharan et al., 1992, Ann. N. Y Acad. Sci. 660:306-309 and Manoharan et al., 1993, Bioorg. Med. Chem. Let. 3:2765-2770); a thiocholesterol (Oberhauser et al., 1992, Nucl. Acids Res. 20: 533-538); an aliphatic chain, e.g., dodecandiol or undecyl residues (Kabanov et al., 1990, FEBS Lett., 259: 327-330 and Svinarchuk et al., 1993, Biochimie, 75: 49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett. 36:3651-3654 and Shea et al., 1990, Nucl. Acids Res. 18: 3777-3783); a polyamine or a polyethylene glycol chain (Mancharan et al., 1995, Nucleosides & Nucleotides 14:969-973); adamantane acetic acid (Manoharan et al., 1995, Tetrahedron Lett. 36:3651-3654); a palmityl moiety (Mishra et al., 1995, Biochim. Biophys. Acta 1264:229-237); or an octadecylamine or hexylamino-carbonyl-t oxycholesterol moiety (Crooke et al., 1996, J. Pharmacol. Exp. Ther., 277: 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes including nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21(1 0):1025-30. Other systems known in the art and regularly developed can be used to target biomolecules of use in the present case and/or complexes thereof to particular target cells of interest.

These targeting moieties or conjugates can include conjugate groups covalently bound to functional groups such as primary or secondary hydroxyl groups. Suitable conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that are capable of enhancing the pharmacodynamic properties include groups that improve uptake, enhance resistance to degradation, and/or strengthen sequence-specific hybridization with the target nucleic acid. Groups that are capable of enhancing the pharmacokinetic properties include groups that improve uptake, distribution, metabolism or excretion of the compounds of the present disclosure. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Longer nucleic acids that are less amenable to chemical synthesis and are generally produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding B-GEn.1, or B-GEn.1.2, or B-GEn.2 is approximately 4kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding B-GEn.1 or B-GEn.2.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech, Axolabs, Bio-Synthesis Inc., Dharmacon and many others. As described by TriLink, for example, 5-Methyl-CTP can be used to impart desirable characteristics such as increased nuclease stability, increased translation or reduced interaction of innate immune receptors with in vitro transcribed RNA. 5′-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation as illustrated in publications by Konmann et al. and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found substituting just one quarter of the uridine and cytidine residues with 2-Thio-U and 5-Methyl-C respectively, resulted in a significant decrease in toll-like receptor (TLR) mediated recognition of the mRNA in mice. By reducing the activation of the innate immune system, these modifications can therefore be used to effectively increase the stability and longevity of the mRNA in vivo; see, e.g., Konmann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs). and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra. Other modifications of nucleic acids described in the art include, for example, the use of polyA tails, the addition of 5′ cap analogs (such as m7G(5′)ppp(5′)G (mCAP)), modifications of 5′ or 3′ untranslated regions (UTRs), or treatment with phosphatase to remove 5′ terminal phosphates-and new approaches are regularly being developed.

A number of compositions and techniques applicable to the generation of modified RNAs for use herein have been developed in connection with the modification of RNA interference (RNAi), including small-interfering RNAs (siRNAs). siRNAs present particular challenges in vivo because their effects on gene silencing via mRNA interference are generally transient, which can require repeat administration. In addition, siRNAs are double-stranded RNAs (dsRNA) and mammalian cells have immune responses that have evolved to detect and neutralize dsRNA, which is often a by-product of viral infection. Thus, there are mammalian enzymes such as PKR (dsRNA-responsive kinase), and potentially retinoic acid-inducible gene I (RIG-1), that can mediate cellular responses to dsRNA, as well as Toll-like receptors (such as TLR3, TLR7 and TLR8) that can trigger the induction of cytokines in response to such molecules; see, e.g., the reviews by Angart et al., Pharmaceuticals (Basel) 6(4): 440-468 (2013); Kanasty et al., Molecular Therapy 20(3): 513-524 (2012); Burnett et al., Biotechnol J. 6(9):1130-46 (2011); Judge and Maclachlan, Hum Gene Ther 19(2):111-24 (2008); and references cited therein.

A large variety of modifications have been developed and applied to enhance RNA stability, reduce innate immune responses, and/or achieve other benefits that can be useful in connection with the introduction of nucleic acids into human cells as described herein; see, e.g., the reviews by Whitehead K A et al., Annual Review of Chemical and Biomolecular Engineering, 2:77-96 (2011); Gaglione and Messere, Mini Rev Med Chem, 10(7):578-95 (2010); Chernolovskaya et al, Curr Opin Mol Ther., 12(2):158-67 (2010); Deleavey et al., Curr Protoc Nucleic Acid Chem Chapter 16:Unit 16.3 (2009); Behlke, Oligonucleotides 18(4):305-19 (2008): Fucini et al., Nucleic Acid Ther 22(3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kale, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O—, 2′-Fluoro, 2′-Hydro) has been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-Methyl, 2′-Fiuoro, 2′-Hydro have been reported to reduce TLR7/TLR8 interaction while generally preserving silencing activity; see, e.g., Judge et al., Mol. Ther. 13:494-505 (2006); and Cekaite et al., J. Mol. Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; see, e.g., Kariko et al., Immunity 23:165-175 (2005).

As is also known in the art, and commercially available, a number of conjugates can be applied to nucleic acids such as RNAs for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

### 6.10. Vectors

The present disclosure provides vectors comprising the nucleic acids of the disclosure, for example as described in Section 6.9. In some embodiments, the nucleic acid comprises a nucleic acid encoding an engineered B-GEn polypeptide as described in Section 6.2. In some embodiments, the engineered B-GEn polypeptide coding sequence is codon-optimized, at least for the portion encoding the nuclease component of the engineered B-GEn polypeptide.

The vector (or nucleotide sequence) may further encode a gRNA.

In some embodiments, the vector comprising the nucleotide sequence may be an expression vector.

In some embodiments the expression vector is production vector for an engineered B-GEn polypeptide, for example useful for the expression/production of the engineered B-GEn polypeptide in host cell. Following expression/production of the engineered B-GEn polypeptide in a host cell, the engineered B-GEn polypeptide can be incorporated into an RNP for nucleofection of a target cell.

Alternatively, the expression vector comprising the nucleotide sequence may be a delivery vector for an engineered B-GEn polypeptide, for example useful for introduction of the engineered B-GEn polypeptide coding sequence into a target cell intended for gene editing. Following expression/production of the engineered B-GEn polypeptide in the target cell, the engineered B-GEn polypeptide, together with guide RNA molecules, is capable of editing the target cell. In some embodiments, a delivery vector further includes coding sequences for the gRNAs. In other embodiments, a separate nucleic acid encoding the gRNA is introduced into the target cell.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic cells include, but are not limited to, the vectors pCTx-1, pCTx-2, and pCTx-3. Other vectors can be used so long as they are compatible with the intended host or target cell.

In some embodiments, an expression vector has one or more transcription and/or translation control elements. Depending on the expression cell/vector system utilized, any of a number of suitable transcription and translation control elements, including constitutive and inducible promoters, transcription enhancer elements, transcription terminators, etc. can be used in the vector. The vector can also contain a ribosome binding site for translation initiation and a transcription terminator.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

For expressing small RNAs, including guide RNAs, various promoters such as RNA polymerase III promoters, including for example U6 and H1, can be advantageous. Thus, such promoters may advantageously be incorporated into delivery vectors. Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

In some embodiments, the vector is a self-inactivating vector that either inactivates the viral sequences or the components of the CRISPR machinery or other elements. Self-inactivating vectors are particularly useful for delivery vectors, to select against cells that retain engineered B-GEn polypeptide coding sequences after gene editing is complete.

In some embodiments, the expression vectors are RNA vectors. In other embodiments, the expression vectors are DNA vectors.

### 6.10.1. RNA Vectors

The expression vectors of the disclosure may be RNA vectors.

Particularly suitable vectors are RNA virus-based virus replicons, such as alphaviruses and Paramyxoviruses. Alphavirus and Paramyxovirus replicons do not involve a DNA intermediate for replication and thus provide a safer alternative to several other commonly used viral vectors including lentiviral and retroviral vectors (Yoshioka et al., 2013, Cell Stem Cell. 13(2):246-54; Yoshioka and Dowdy, 2017, PLOS ONE 12:e0182018). Alphaviruses are lipid-enveloped, positive-sense RNA viruses, which constitute a genus of more than 30 viruses in the Togaviridae family, including Eastern, Western, and Venezuelan equine encephalitis viruses (EEEV, WEEV, and VEEV, respectively), chikungunya (CHIK), Sindbis, Ross River, and O'nyong-nyong viruses, among others. Sendai viruses (SeV) are enveloped, single-stranded negative-sense Paramyxoviruses that replicate episomally in a host cell cytoplasm.

Accordingly, in some embodiments, an RNA vector is derived from an RNA virus, such as an alphavirus, a paramyxovirus, a flavivirus, a rhabdovirus, a measles virus, or a picornavirus.

In some embodiments, the RNA vector is a single stranded RNA replicon. In some embodiments, the single stranded RNA replicon is a positive strand. In some other embodiments, the single stranded RNA replicon is a negative strand. In some embodiments the RNA vector comprises one or more coding sequences for one or more engineered B-GEn polypeptides and a self-replication element.

RNA replicons of the disclosure typically include regulatory elements, a subgenomic (SG) promoter, operably linked to the engineered B-GEn polypeptide coding sequence(s). The sequences comprising engineered B-GEn polypeptide coding sequence are typically flanked by 5 and 3′ UTR sequence, and the 3′ UTR sequence is typically followed by a polyadenylation signal.

The RNA vector construct may be produced from a DNA template (, a DNA plasmid construct). By way of example, the RNA construct may be transcribed from a DNA template by using a SP6 or T7 in vitro transcription kit.

RNA vectors are particularly useful as delivery vectors.

### 6.10.2. DNA Vectors

In some embodiments, an expression vector of the disclosure is a DNA vector. The present disclosure provides two types of DNA vectors: (1) a DNA vector that is a production vector or delivery vector and (2) a DNA vector from which an RNA vector of the disclosure (, as described in Section 6.10.1) can be transcribed, as described in Section 6.10.2.2. The DNA vector from which an RNA replicon of the disclosure can be transcribed is sometimes referred to herein as a “template vector”.

In some embodiments, the DNA vector of the disclosure is a nonintegrating DNA vector. For example, the vector can be an episomal vector. For instance, a number of DNA viruses, such as adenoviruses, Simian vacuolating virus 40 (SV40), bovine papilloma virus (BPV), or budding yeast ARS (Autonomously Replicating Sequences)-containing plasmids may be used without genomic integration.

In some embodiments, the DNA vectors of the disclosure include an origin of replication. Examples of origins of replications that may be incorporated into a DNA vector of the disclosure include the replication origin of a lymphotropic herpes virus, a gammaherpesvirus, an adenovirus, a bovine papilloma virus, or a yeast. In some embodiments, the replication origin is from a lymphotropic herpes virus or a gammaherpesvirus corresponding to oriP of EBV, as a self-replication element. In some embodiments, the lymphotropic herpes virus is Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gammaherpesvirus.

In certain embodiments, a vector of the disclosure comprises a replication origin of EBV, OrIP. OriP is the site at or near which DNA replication initiates and is composed of two cis-acting sequences approximately 1 kilobase pair apart known as the family of repeats (FR) and the dyad symmetry (DS). FR is composed of 21 imperfect copies of a 30 bp repeat and contains 20 high affinity EBNA-1-binding sites. When FR is bound by EBNA-1, it both serves as a transcriptional enhancer of promoters in cis up to 10 kb away. DS is sufficient for initiation of DNA synthesis in the presence of EBNA-1 and initiation occurs either at or near DS.

One or more of the expression cassettes in a replicating DNA vector may further comprise a nucleotide sequence encoding a trans-acting factor that binds to the replication origin to replicate an extra-chromosomal template. Alternatively or additionally, the somatic cell may express such a trans-acting factor.

In other embodiments, the DNA vectors of the disclosure lack an origin of replication.

The DNA vectors of the disclosure typically comprise one or more promoters, e.g., SP6 or T7, to drive expression of the engineered B-GEn polypeptide in the case of DNA vectors that are intended to be production vectors or expression of an RNA replicons in the case of DNA vectors that are intended to be template vectors.

### 6.10.2.1. Expression Vectors

In some embodiments, the expression vector is a DNA vector comprising expression cassettes for expression of one or more proteins of interest, operably linked to a regulatory element comprising a promoter suitable for driving expression of the engineered B-GEn polypeptide in the cell type of interest. Examples of promoters suitable for driving expression of proteins in mammalian cells include cytomegalovirus (CMV) promoters, EF1a promoters, SV40 promoter, Ubc promoter, human beta actin promoters, PGK1 promoters and CAG promoters.

DNA vectors that are used for direct expression of an engineered B-GEn polypeptide (rather than as a template for expression of an RNA vector as described in Section 6.10.2.2) need not include RNA replicon self-replication sequences, for example the nsP1-nsP4 proteins of VEEV or NP, P and L proteins of Sendai virus.

In some embodiments, a DNA expression vector is a non-replicating DNA vector. In some embodiments, a DNA expression vector is a replicating DNA vector.

### 6.10.2.2. Template Vectors

The DNA vectors of the disclosure may also serve as templates for transcription of an RNA replicon as described herein. Thus, the “expression cassettes” included in template vectors are intended for transcription from the RNA replicon produced by transcription of the RNA replicon.

Accordingly, the template vectors of the disclosure comprise a nucleotide sequence encoding an RNA replicon as described herein under the control of a regulatory element, the SP6 or T7 promoter.

In some embodiments, a template DNA vector is a non-replicating DNA vector. In some embodiments, a template DNA vector is a replicating DNA vector.

In some embodiments, the template vectors are used for in vitro transcription of an RNA replicon that is subsequently introduced into a cell to drive expression of the engineered B-GEn polypeptide.

### 6.10.3. Viral Vectors

A recombinant adeno-associated virus (AAV) vector may be used for delivery. Known techniques to produce rAAV particles in the art is to provide a cell with a polynucleotide to be delivered between two AAV invert terminal repeats (ITRs), AAV rep and cap genes and helper virus functions. Production of rAAV requires that the following components are present within a single cell (denoted herein as a packaging cell): a polynucleotide of interest between two ITRs, AAV rep and cap genes separate from (i.e., not in) the AAV genome, and helper virus functions. The AAV rep and cap genes may be from any AAV serotype for which recombinant virus can be derived and may be from a different serotype of AAV than that of ITRs on a packaged polynucleotide, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, WO 01/83692.

| AAV Serotype | Genbank Accession No. |
|---|---|
| AAV-1 | NC_002077.1 |
| AAV-2 | NC_001401.2 |
| AAV-3 | NC_001729.1 |
| AAV-38 | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | 00813647.1 |
| AAV-13 | EU285562.1 |

A method of generating a packaging cell is to create a cell line that stably expresses all the necessary components for AAV particle production. For example, a plasmid (or multiple plasmids) comprising a polynucleotide of interest between AAV ITRs, AAV rep and cap genes separate from the AAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. Sci. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

AAV vector serotypes used for transduction are dependent on target cell types. For example, the following exemplary cell types are known to be transduced by the indicated AAV serotypes among others.

| Tissue/Cell Type | Serotype |
|---|---|
| Liver | AAV8, AAV9 |
| Skeletal muscle | AAV1, AAV7, AAV6, AAV8, AAV9 |
| Central nervous system | AAV5, AAV1, AAV4 |
| RPE | AAV5, AAV4 |
| Photoreceptor cells | AAV5 |
| Lung | AAV9 |
| Heart | AAV8 |
| Pancreas | AAV8 |
| Kidney | AAV2 |

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

### 6.11. Host Cells and Recombinant Expression

In some embodiments, host cells may be employed to express gRNAs, sgRNAs, or the engineered B-GEn polypeptides of the disclosure. Suitable host cells include naturally occurring cells; genetically modified cells (e.g., cells genetically modified in a laboratory), and cells manipulated in vitro in any way. In some embodiments, a host cell is isolated.

The host cell can be a eukaryote or prokaryote and includes, for example, yeast (such as Pichia pastoris or Saccharomyces cerevisiae), bacteria (such as E. coli or Bacillus subtilis), insect Sf9 cells (such as baculovirus-infected SF9 cells) or mammalian cells (such as Human Embryonic Kidney (HEK) cells, Chinese hamster ovary cells, HeLa cells, human 293 cells and monkey COS-7 cells).

Host cells may be from established cell lines, or they may be primary cells, where “primary cells”, “primary cell lines”, and “primary cultures” are used interchangeably herein to refer to cells and cells cultures that have been derived from a subject and allowed to grow in vitro for a limited number of passages, e.g., splittings, of the culture. For example, primary cultures include cultures that may have been passaged 0 times, 1 time, 2 times, 4 times, 5 times, 10 times, or 15 times, but not enough times go through the crisis stage. Primary cell lines can be maintained for fewer than 10 passages in vitro. In some embodiments, host cells are PSCs (e.g., iPSCs, or ESCs), or PSC-derived cells (e.g., PSC-derived neurons, PSC-derived microglial cells, PSC-derived cardiomyocytes, PSC-derived cells of the eye).

If the cells are primary cells, such cells may be harvested from an individual by any suitable method. An appropriate solution may be used for dispersion or suspension of the harvested cells. The harvested cells may be used immediately, or they may be stored, frozen, for long periods of time, being thawed and capable of being reused. In such cases, the cells will generally be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells.

### 6.12. Target Cells

In some embodiments, the B-GEn CRISPR-Cas system is introduced into target cells or populations of target cells. Methods for introducing proteins and nucleic acids to target cells are described further in Section6.13.

The target cells and target cell populations of the disclosure can be cells in which gene editing by the systems of the disclosure has taken place, or cells in which the components of a system of the disclosure have been introduced or expressed but gene editing has not taken place, or a combination thereof. In various embodiments, a cell population can comprise, for example, a population in which at least 1%, at least 5%, at least 10%, at least 15%, at least 20%, at least 30%, at least 40%, at least 50%, at least 60%, or at least 70% of the cells have undergone gene editing by a system of the disclosure.

In some embodiments, the methods of the disclosure may be employed to induce transcriptional modulation in mitotic or post-mitotic cells in vivo and/or ex vivo and/or in vitro. In some embodiments, the methods of the disclosure may be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual).

Because the guide RNA provides specificity by hybridizing to target DNA, a mitotic and/or post-mitotic cell can be any of a variety of target cell, where suitable target cells include, but are not limited to, a bacterial cell; an archaeal cell; a single-celled eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell; an animal cell; a cell from an invertebrate animal (e.g., an insect, a cnidarian, an echinoderm, a nematode, etc.); a eukaryotic parasite (e.g., a malarial parasite, e.g., Plasmodium fakiparum; a helminth; etc.); a cell from a vertebrate animal (e.g., fish, amphibian, reptile, bird, mammal); a mammalian cell, e.g., a rodent cell, a human cell, a non-human primate cell, etc. In some embodiments, the target cell can be any human cell. Suitable target cells include naturally occurring cells; genetically modified cells (e.g., cells genetically modified in a laboratory, e.g., by the “hand of man”); and cells manipulated in vitro in any way. In some embodiments, a target cell is isolated.

Any type of cell may be of interest as a host cell or target cell (e.g., a stem cell, e.g., an embryonic stem (ES) cell, an induced pluripotent stem cell (iPSC), a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines, or they may be primary cells, where “primary cells”, “primary cell lines”, and “primary cultures” are used interchangeably herein to refer to cells and cells cultures that have been derived from a subject and allowed to grow in vitro for a limited number of passages, e.g., splittings, of the culture. For example, primary cultures include cultures that may have been passaged 0 times, 1 time, 2 times, 4 times, 5 times, 10 times, or 15 times, but not enough times go through the crisis stage. Primary cell lines can be maintained for fewer than 10 passages in vitro. Target cells are, in some embodiments, unicellular organisms, or are grown in culture. In some embodiments, a host cell is the same as a target cell. In some embodiments, a target cell is modified to become another cell type such that the resultant host cell is different from the target cell. As an example, a target cell may be a PSC (e.g., iPSC) which is then differentiated into a PSC-derived cell (such as a PSC-derived neuron) such that the host cell is a neuron.

If the cells are primary cells, such cells may be harvested from an individual by any suitable method. For example, leukocytes may be suitably harvested by apheresis, leukocytapheresis, density gradient separation, etc., while cells from tissues such as skin, muscle, bone marrow, spleen, liver, pancreas, lung, intestine, stomach, etc. are most suitably harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., suitably supplemented with fetal calf serum or other naturally occurring factors, in conjunction with an acceptable buffer at low concentration, e.g., from 5-25 mM. Suitable buffers include HEPES, phosphate buffers, lactate buffers, etc. The cells may be used immediately, or they may be stored, frozen, for long periods of time, being thawed and capable of being reused. In such cases, the cells will generally be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells.

6.12.1. Induced Pluripotent Stem Cells (iPSCs)

In some embodiments, the target cells are induced pluripotent stem cells (iPSCs), which are the starting point for the potential generation of large numbers of a specific cell type that can be delivered for regenerative medicine in patients with many different diseases. Differentiation, in the context of iPSC, is the process of lineage specification using cell specific protocols, starting with an iPSC. The iPSCs of the disclosure can be differentiated into a cell type of interest for cell therapy, including cells in the endoderm (e.g., lung, thyroid, or pancreatic cells, or progenitors thereof), ectoderm (e.g., skin, neuronal, or pigment cells, or progenitors thereof) and mesoderm (e.g., cardiac cells, skeletal muscle cells, red blood cells, smooth muscle cells, or progenitors thereof) lineages.

In some embodiments, an iPSC of the disclosure is differentiated into a cardiac cell. In various embodiments, the cardiac cell is a cardiac progenitor cell or a mature or immature (atrial or ventricular) cardiomyocyte.

In other embodiments, an iPSC of the disclosure is differentiated into an oligodendrocyte progenitor cells or an oligodendrocyte.

In other embodiments, an iPSC of the disclosure is differentiated into a neural lineage cell, for example a neural crest cells, an astrocyte, a dopaminergic neuron progenitor cell, a dopaminergic neuron cells, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron.

In other embodiments, an iPSC of the disclosure is differentiated into a photoreceptor cell, a photoreceptor precursor cell, a retinal pigmented epithelium cell, a neural retinal cell, or a neural retinal progenitor cell.

In further embodiments, an iPSC of the disclosure is differentiated into a microglial cell or a microglial progenitor cell.

In further embodiments, an iPSC of the disclosure is differentiated into a macrophage.

In further embodiments, an iPSC of the disclosure is differentiated into an enteric progenitor cell or an enteric cell.

In some embodiments, the iPSCs may be genetically engineered (e.g., to produce a functional protein that is defective in a patient, to produce a therapeutic protein, to include a shutoff switch, or to evade immune detection, thereby supporting allogeneic applications) prior to differentiation into a cell type of interest.

6.13. Methods of Introducing Nucleic Acids into Host and Target Cells

In some embodiments, the methods of the disclosure include involve introducing into a host or target cell (or a population of host or target cells) one or more nucleic acids comprising a nucleotide sequence encoding a guide RNA and/or a codon-optimized nucleotide sequence encoding an engineered B-GEn polypeptide. In some embodiments, a target cell (e.g., a cell comprising DNA that is targeted by a guide RNA for editing by an engineered B-GEn polypeptide) is in vitro. In some embodiments, a target cell is in vivo. In some embodiments, the nucleotide sequence encoding a guide RNA and/or an engineered B-GEn polypeptide is operably linked to an inducible promoter. In some embodiments, a nucleotide sequence encoding a guide RNA and/or an engineered B-GEn polypeptide is operably linked to a constitutive promoter.

A guide RNA, or a nucleic acid comprising a nucleotide sequence encoding same, can be introduced into a host or target cell by any of a variety of well-known methods. Similarly, where a method involves introducing into a host or target cell a nucleic acid comprising a codon-optimized nucleotide sequence encoding an engineered B-GEn polypeptide, such a nucleic acid can be introduced into a host or target cell by any of a variety of well-known methods. Guide nucleic acids (RNA or DNA) and/or engineered B-GEn polypeptide-encoding nucleic acids (RNA or DNA) can be delivered by viral or non-viral delivery vehicles known in the art.

Methods of introducing a nucleic acid into a host or target cell are known in the art, and any known method can be used to introduce a nucleic acid (e.g., an expression construct) into a stem cell or progenitor cell. Suitable methods include, e.g., viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: 50169-409X(12)00283-9. doi: 10.1016/j.addr.2012.09.023), and the like, including but not limiting to exosome delivery.

Polynucleotides may be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other nucleic acids).

Suitable systems and techniques for delivering a nucleic acid of the disclosure (e.g., mRNA and sgRNA) for gene editing include lipid nanoparticles (LNPs). As used herein, the term “lipid nanoparticles” includes liposomes irrespective of their lamellarity, shape or structure and lipoplexes as described for the introduction of nucleic acids and/or polypeptides into cells. These lipid nanoparticles can be complexed with biologically active compounds (e.g., nucleic acids and/or polypeptides) and are useful as in vivo delivery vehicles. In general, any method known in the art can be applied to prepare the lipid nanoparticles comprising one or more nucleic acids of the present disclosure and to prepare complexes of biologically active compounds and said lipid nanoparticles. Examples of such methods are widely disclosed, e.g., in Biochim Biophys Acta 1979, 557:9; Biochim et Biophys Acta 1980, 601:559; Liposomes: A practical approach (Oxford University Press, 1990); Pharmaceutica Acta Helvetiae 1995, 70:95; Current Science 1995, 68:715; Pakistan Journal of Pharmaceutical Sciences 1996, 19:65; Methods in Enzymology 2009, 464:343). Particularly suitable systems and techniques for preparing LNP formulations comprising one or more nucleic acids and/or polypeptides of the present disclosure include, but are not limited to, those developed by Intellia (see e.g., WO2017173054A1), Alnylam (see, e.g., WO2014008334A1), Modernatx (see., e.g., WO2017070622A1 and WO2017099823A1), TranslateBio, Acuitas (see, e.g., WO2018081480A1), Genevant Sciences, Arbutus Biopharma, Tekmira, Arcturus, Merck (see, e.g., WO2015130584A2), Novartis (see, e.g., WO2015095340A1), and Dicerna; all of which are herein incorporated by reference in their entireties.

Suitable nucleic acids comprising nucleotide sequences encoding an engineered B-GEn polypeptide and/or a guide RNA include expression vectors. In some embodiments, the expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc. Suitable expression vectors include, but are not limited to, viral vectors (e.g. viral vectors based on vaccinia virus; poliovirus; adenovirus (see, e.g. Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:10881097, 1999; WO 94/12649, WO 93/03769; WO 93/19191; WO 94/28938; WO 95/11984 and WO 95/00655); adeno-associated virus (see, e.g. Ali et al., Hum Gene Ther 9:81 86,1998, Flannery et al., PNAS 94:6916 6921, 1997; Bennett et al., Invest Opthalmol Vis Sci 38:2857 2863, 1997; Jomary et al., Gene Ther 4:683-690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (see, e.g. Miyoshi et al., PNAS 94:10319 23, 1997; Takahashi et al., J Virol 73:7812 7816, 1999); a retroviral vector (e.g. Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, a B-GEn ribonucleoprotein comprising a B-GEn endonuclease and an sgRNA is delivered to target cells through nucleofection, which is a method of delivering nucleic acid to the cells by the use of cell-specific reagents and electrical parameters to create transient small pores in the cell membrane.

Engineered B-GEn Type V CRISPR-Cas systems may be delivered into target cells by delivery vectors, such as viral vectors. Engineered B-GEn Type V CRISPR-Cas systems may also be delivered into target cells by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011).

In some embodiments, an engineered B-GEn Type V CRISPR-Cas system is delivered into target cells via the use of delivery vectors as described in Section 6.10.

### 6.14. Pharmaceutical Compositions

Also disclosed herein are pharmaceutical formulations and medicaments comprising a B-GEn protein, gRNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS) and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type II Cas protein in a pharmaceutically effective amount sufficient to edit a gene in a cell. The pharmaceutical compositions can be formulated for medical and/or veterinary use.

In some embodiments, the B-GEn endonuclease complexes may be introduced into host cells, e.g., iPSCs, to produce genetically modified cells that can be reintroduced into an individual. The iPSC-derived cells described herein may be provided in a pharmaceutical composition containing the cells and a pharmaceutically acceptable carrier. The pharmaceutically acceptable carrier may be cell culture medium that optionally does not contain any animal-derived component. For storage and transportation, the cells may be cryopreserved at <−70° C. (e.g., on dry ice or in liquid nitrogen). Prior to use, the cells may be thawed, and diluted in a sterile cell medium that is supportive of the cell type of interest.

The cells may be administered into the patient systemically (e.g., through intravenous injection or infusion), or locally (e.g., through direct injection to a local tissue, e.g., the heart, the brain, and a site of damaged tissue). Various methods are known in the art for administering cells into a patient's tissue or organs, including, without limitation, intracoronary administration, intramyocardial administration, transendocardial administration, or intracranial administration.

A therapeutically effective number of iPSC-derived cells are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose. In some embodiments, at least 10³ (e.g., at least 10⁴, at least 10⁵, at least 10⁶, at least 10⁷, at least 10⁸, at least 10⁹, at least 10¹⁰, at least 10¹¹, or at least 10¹²) cells are administered to a subject at a time in one or more sites. In some embodiments, 10³-10¹³ (e.g., 10³-10⁴, 10³-10⁵, 10³-10⁶, 10³-10⁷, 10³-10⁸, 10³-10⁹, 10³-10¹⁰, 10³-10¹¹, 10³-10¹², 10⁶-10⁷, 10⁶-10⁸, 10⁶-10⁹, 10⁶-10¹⁰, 10⁶-10¹¹, 10⁶-10¹², 10⁹-10¹⁰, 10⁹-10¹¹, 10⁹-10¹²) cells are administered to a subject at a time in one or more sites. In some embodiments, more than 10¹² (e.g., more than 10¹², more than 10¹³, more than 10¹⁴, more than 10¹⁵, more than 10¹⁶, more than 10¹⁷, more than 10¹⁸ or more) cells are administered to a subject at a time at one or more sites.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. A fusion polypeptide comprising:
(a) a nuclease sequence:
   (i) as defined in Section 4.3;
   (ii) which is an amino acid sequence having at least 80% sequence identity to SEQ ID NO4 (B-GEn.1), SEQ ID NO:5 (B-GEn.1.2), or SEQ ID NO:6 (B-GEn.2);
   (iii) which is an amino acid sequence having at least 80% sequence identity to the nuclease domain of SEQ ID NO:4 (B-GEn.1), SEQ ID NO:5 (B-GEn.1.2), or SEQ ID NO:6 (B-GEn.2);
   (iv) which is an amino acid sequence that differs from the amino acid sequence of SEQ ID NO:4 (B-GEn.1), SEQ ID NO:5 (B-GEn.1.2), or SEQ ID NO:6 (B-GEn.2) by no more than 25 amino acids; or
   (v) any combination of two, three or all four of (i)-(iv),
(b) a first nuclear localization signal (“NLS”) sequence C-terminal to the nuclease sequence.

2. The fusion polypeptide of embodiment 1, which further comprises a first linker sequence between the nuclease sequence and the first NLS sequence.

3. The fusion polypeptide of embodiment 1 or embodiment 2, which further comprises a second NLS sequence C-terminal to the first NLS sequence.

4. The fusion polypeptide of embodiment 3, which comprises a linker sequence between the first NLS sequence and the second NLS sequence.

5. The fusion polypeptide of embodiment 3 or embodiment 4, which further comprises a third NLS sequence C-terminal to the second NLS sequence.

6. The fusion polypeptide of embodiment 5, which comprises a linker sequence between the second NLS sequence and the third NLS sequence.

7. The fusion polypeptide of embodiment 5 or embodiment 6, which further comprises a fourth NLS sequence C-terminal to the third NLS sequence.

8. The fusion polypeptide of embodiment 7, which comprises a linker sequence between the third NLS sequence and the fourth NLS sequence.

9. The fusion polypeptide of any one of embodiments 1 to 8, which lacks a NLS sequence N-terminal to the nuclease sequence.

10. The fusion polypeptide of any one of embodiments 1 to 8, which comprises a NLS sequence N-terminal to the nuclease sequence.

11. The fusion polypeptide of embodiment 10, which comprises a linker sequence between the NLS sequence N-terminal to the nuclease sequence and the nuclease sequence.

12. The fusion polypeptide of any one of embodiments 1 to 11, wherein each NLS sequence is independently selected from an NLS sequence set forth in Table 3.

13. The fusion polypeptide of any one of embodiments 1 to 12, wherein each linker sequence is independently selected from a nuclease sequence set forth in Table 4.

14. The fusion polypeptide of any one of embodiments 1 to 13, wherein the nuclease sequence has at least 85% sequence identity to the amino acid sequence of any one of SEQ ID NO:4, SEQ ID NO:5, or SEQ ID NO:6.

15. The fusion polypeptide of any one of embodiments 1 to 13, wherein the nuclease sequence has at least 90% sequence identity to the amino acid sequence of any one of SEQ ID NO:4, SEQ ID NO:5, or SEQ ID NO:6.

16. The fusion polypeptide of any one of embodiments 1 to 13, wherein the nuclease sequence has at least 95% sequence identity to the amino acid sequence of any one of SEQ ID NO:4, SEQ ID NO:5, or SEQ ID NO:6.

17. The fusion polypeptide of any one of embodiments 1 to 13, wherein the nuclease sequence has at least 98% sequence identity to the amino acid sequence of any one of SEQ ID NO:4, SEQ ID NO:5, or SEQ ID NO:6.

18. The fusion polypeptide of any one of embodiments 1 to 13, wherein the nuclease sequence has at least 99% sequence identity to the amino acid sequence of any one of SEQ ID NO:4, SEQ ID NO:5, or SEQ ID NO:6.

19. The fusion polypeptide of any one of embodiments 1 to 13, wherein the nuclease sequence has at least 99.5% sequence identity to the amino acid sequence of any one of SEQ ID NO:4, SEQ ID NO:5, or SEQ ID NO:6.

20. The fusion polypeptide of any one of embodiments 1 to 19, which comprises or consists of the amino acid sequence of SEQ ID NO:1.

21. The fusion polypeptide of any one of embodiments 1 to 19, which comprises or consists of the amino acid sequence of SEQ ID NO:2.

22. The fusion polypeptide of any one of embodiments 1 to 19, which comprises or consists of the amino acid sequence of SEQ ID NO:3.

23. A nucleic acid comprising a nucleotide sequence encoding the fusion polypeptide of any one of embodiments 1 to 22.

24. The nucleic acid of embodiment 23, wherein the nucleotide sequence encoding the fusion polypeptide of any one of embodiments 1 to 22 is operably linked to a promoter.

25. The nucleic acid of embodiment 23 or embodiment 24, wherein the nucleic acid further encodes a guide RNA.

26. The nucleic acid of embodiment 25, wherein the guide RNA comprises any one of the nucleotide sequences set forth in Table 6.

27. The nucleic acid of embodiment 25 or embodiment 26, wherein the guide RNA is capable of targeting a PAM sequence as set forth in Table 7.

28. The nucleic acid of any one of embodiments 25 to 27, wherein the guide RNA comprises any one of the nucleotide sequences set forth in Table 5.

29. The nucleic acid of any one of embodiments 23 to 28, which is in the form of a vector.

30. The nucleic acid of embodiment 29, wherein the vector is an expression vector.

31. The nucleic acid of embodiment 30, wherein the expression vector is a production vector.

32. The nucleic acid of embodiment 30, wherein the expression vector is a delivery vector.

33. The nucleic acid of any one of embodiments 29 to 32, wherein the vector is an RNA vector.

34. The nucleic acid of any one of embodiments 29 to 32, wherein the vector is a DNA vector.

35. The nucleic acid of embodiment 34, wherein the DNA vector is a plasmid.

36. A cell comprising the nucleic acid of any one of embodiments 23 to 35.

37. A cell engineered to express a nucleotide sequence encoding the fusion polypeptide of any one of embodiments 1 to 22.

38. The cell of embodiment 36 or embodiment 37, which is a eukaryotic cell.

39. The cell of embodiment 38, which is an insect cell.

40. The cell of embodiment 38, which is a mammalian cell.

41. A method of producing a fusion polypeptide according to any one of embodiments 1 to 22, comprising culturing the cell of any one of embodiments 36 to 40 under conditions in which the fusion polypeptide is produced.

42. The method of embodiment 41, which further comprises isolating and/or purifying the fusion polypeptide.

43. A composition comprising:
(a) a fusion polypeptide according to any one of embodiments 1 to 22; and
(b) a guide RNA.

44. The composition of embodiment 43, wherein the guide RNA comprises any one of the nucleotide sequences set forth in Table 6.

45. The composition of embodiment 43 or embodiment 44, wherein the guide RNA is capable of targeting a PAM sequence as set forth in Table 7.

46. The composition of any one of embodiments 43 to 45, wherein the guide RNA comprises any one of the nucleotide sequences set forth in Table 5.

47. The composition of any one of embodiments 43 to 46, which is a ribonucleoprotein complex.

48. A method of editing the genome of a cell, comprising introducing into the cell:
(a) the fusion polypeptide of any one of embodiments 1 to 22; and
(b) a guide RNA.

49. A method of editing the genome of a cell, comprising introducing into the cell one or more nucleic acids encoding:
(a) the fusion polypeptide of any one of embodiments 1 to 22; and
(b) a guide RNA,
optionally wherein at least one of the one or more nucleic acids is a nucleic acid according to any one of embodiments 23 to 35.

50. The method of embodiment 48 or embodiment 49, wherein the guide RNA comprises any one of the nucleotide sequences set forth in Table 6.

51. The method of any one of embodiments 48 to 50, wherein the guide RNA is capable of targeting a PAM sequence as set forth in Table 7.

52. The method of any one of embodiments 48 to 51, wherein the guide RNA comprises any one of the nucleotide sequences set forth in Table 5.

53. A method of editing the genome of a cell, comprising introducing into the cell the composition of any one of embodiments 43 to 47.

54. The method of any one of embodiments 48 to 53, wherein the cell is a stem cell.

55. The method of embodiment 54, wherein the stem cell is an induced pluripotent stem cell (iPSC).

56. The method of any one of embodiments 48 to 55, wherein the stem cell is a mammalian stem cell.

57. The method of embodiment 56, wherein the mammalian stem cell is a human stem cell.

58. A stem cell comprising:
(a) the composition of any one of embodiments 43 to 44; or
(b) a nucleic acid according to any one of any one of embodiments 23 to 35.

59. The stem cell of embodiment 58, which is an induced pluripotent stem cell (iPSC).

60. The stem cell of embodiment 58 or embodiment 59, which is a mammalian stem cell.

61. The stem cell of embodiment 60, which is a human stem cell.

### 8. EXAMPLES

### 8.1. Example 1: Effect of NLS Sequence and Orientation on Nuclear Transport of B-GEn Endonucleases

An assay is developed to screen B-GEn nuclease-nuclear localization signals (NLSs) fusions to identify optimal NLS sequences and orientations for efficient nuclear transport of B-GEn endonucleases. NLSs are short, positively charged peptides that mediate transport of proteins from the cytoplasm to the nucleus via nuclear pore complexes. Hence, fusing endonuclease coding sequences to one or more NLS sequences can facilitate their delivery to the nucleus. Generally, endonucleases can be tagged on either the N- or C-terminus, at both termini, and at certain interdomain regions that allow the NLS to bind to its cognate receptor within the nuclear pore complex.

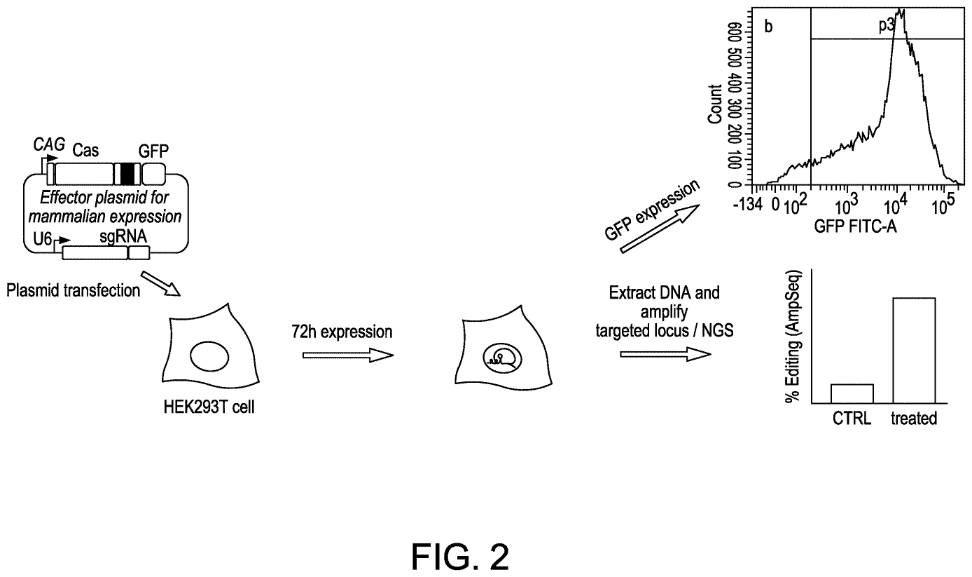

A variety of NLS sequences are reported in the literature and nonlimiting examples are listed in Table 3 in Section 6.4. An assay setup depicted in FIG. 2 is used to screen NLS sequences that are fused either to the N-terminus or to the C-terminus of a Cas endonuclease. Briefly, plasmids comprising a B-GEn sequence fused to an NLS sequence at either its N-terminus or C-terminus, a downstream reporter gene sequence, e.g., GFP sequence, and an sgRNA sequence are transfected into HEK293-T cells using standard protocols. Cells are harvested 72 hours after transfection. A portion of the harvested cells are used to confirm transfection efficiency via assessing the expression of the reporter gene. The rest of the harvested cells are used to prepare DNA extracts in order to test percent gene editing at targeted loci. Individual NLS sequence configurations will be assessed in both N-terminus and C-terminus orientations relative to the B-GEn sequence.

### 8.2. Example 2: Designing B-GEn.2 Variant Constructs

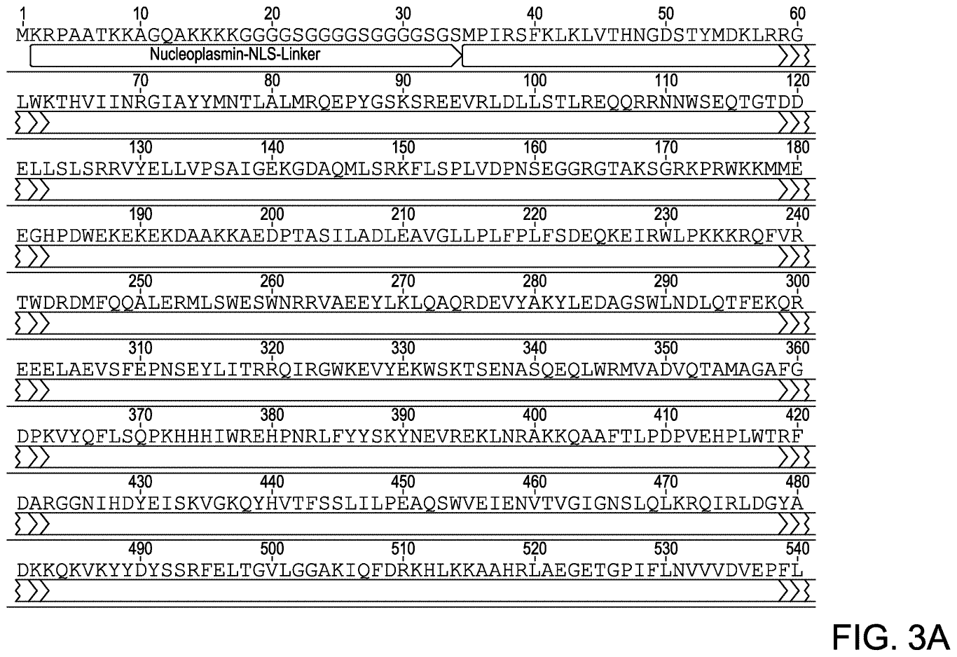

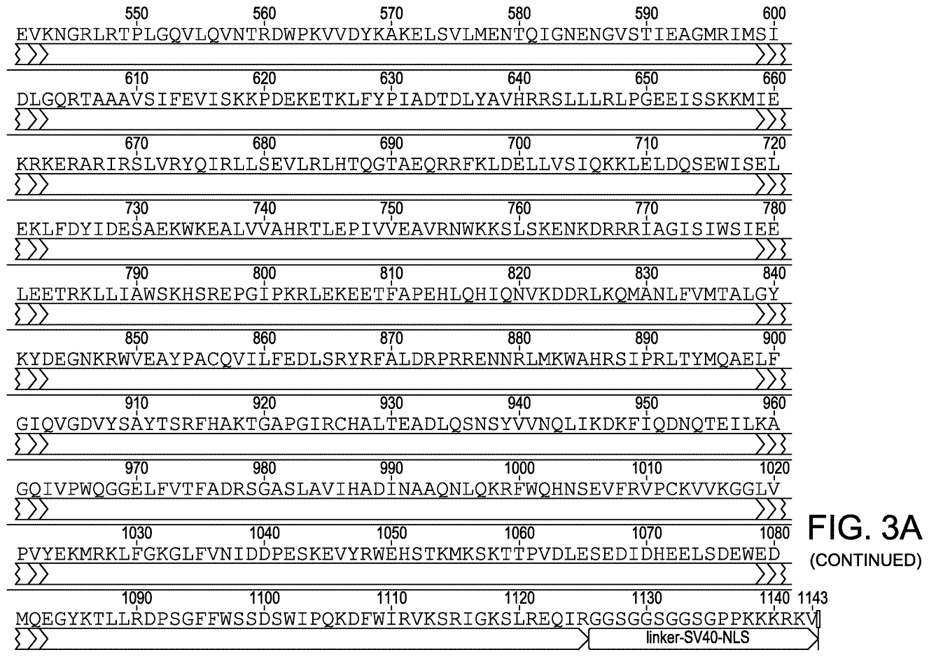

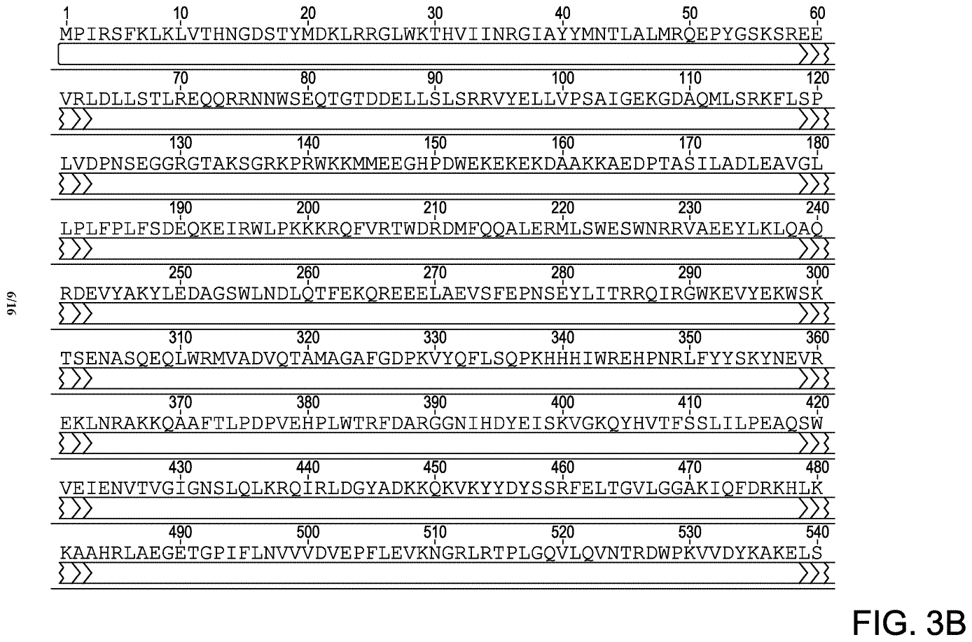

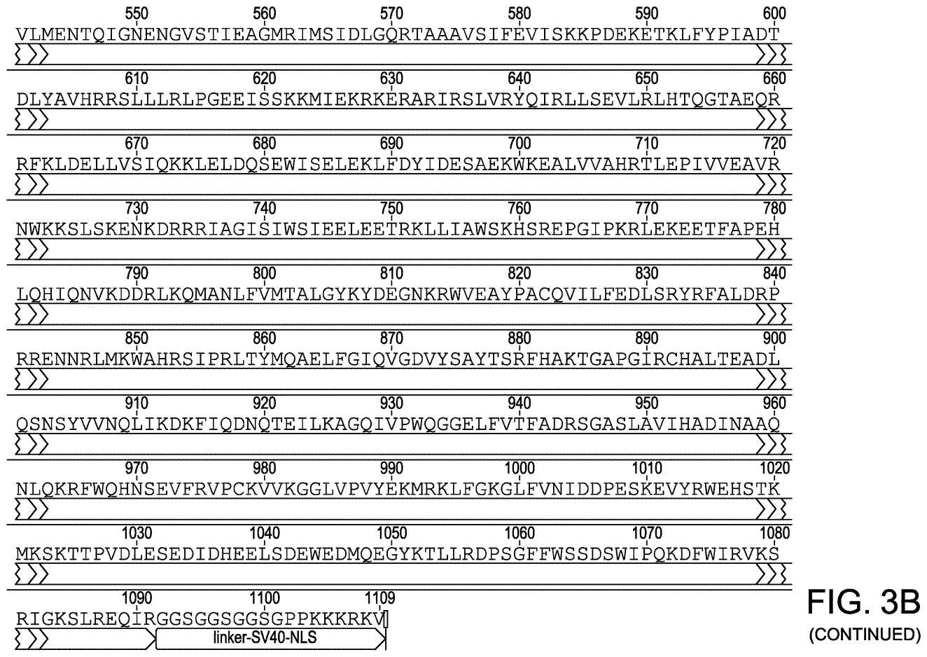

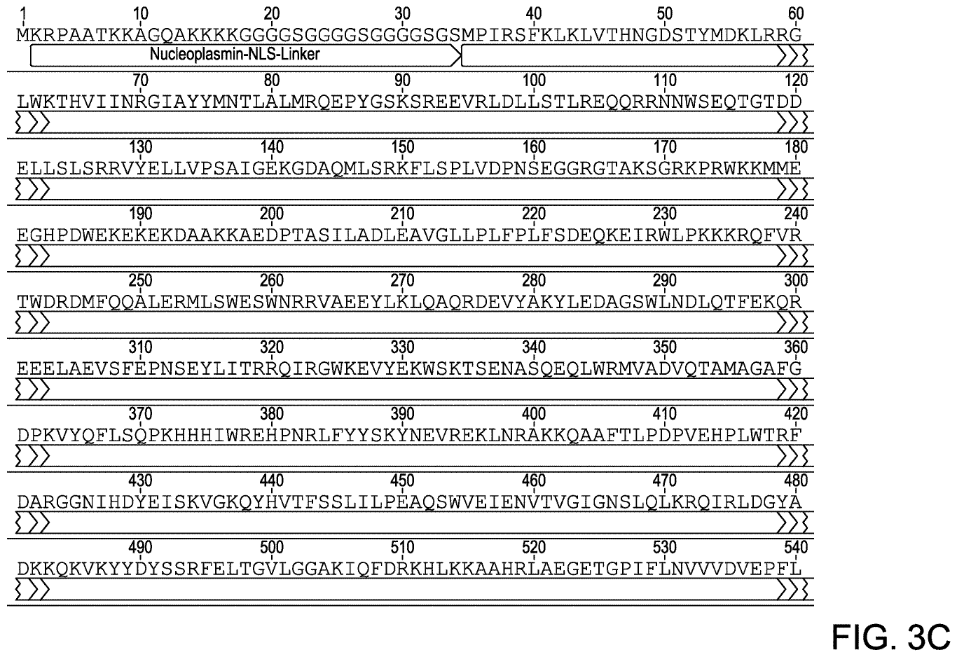

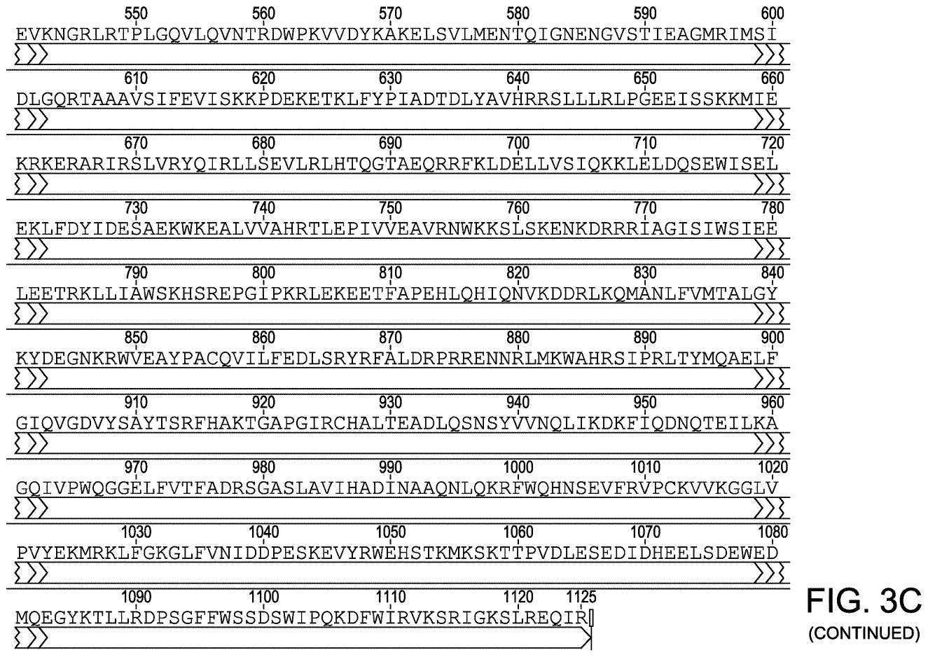

A study was conducted to compare B-GEn.2 constructs comprising two NLS sequences flanking the B-GEn.2 sequence: a Nucleoplasmin NLS located at the N-terminus and an SV40 NLS located at the C-terminus of B-GEn.2 (FIGS. 1A-1 and 3A) with variants from which the N-terminal NLS, C-terminal NLS, or both NLS's were deleted (FIGS. 1A2-1A4). NLS deletion at the N-terminus yielded a construct, B-GEn.2 Ndel, which included an SV40 NLS only on its C-terminus (FIG. 3B). NLS deletion at the C-terminus yielded a construct, B-GEn.2 Cdel, which included only a Nucleoplasmin NLS on its N-terminus (FIG. 3C). The deletion of both NLS sequences yielded a construct, B-GEn.2 Full-del, with no NLS sequences.

### 8.3. Example 3: Expression and Purification of B-GEn.2 Variants

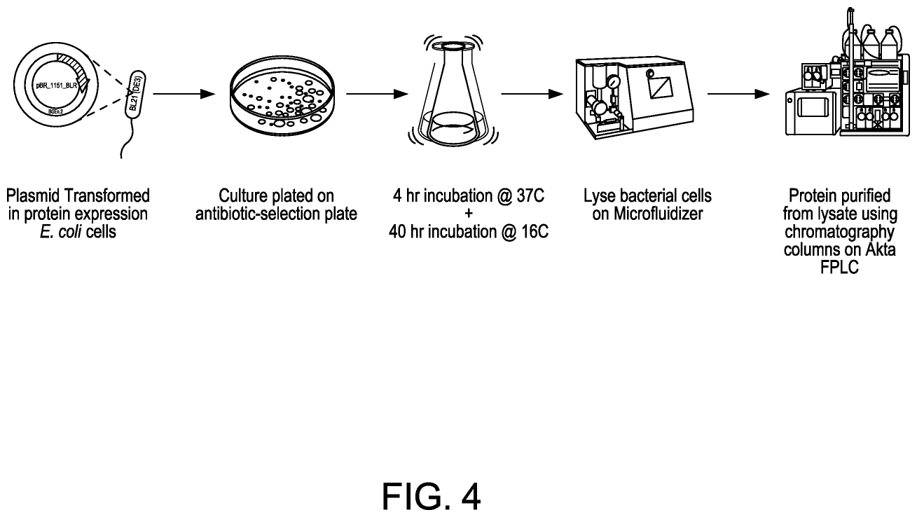

The expression and purification workflow of B-GEn.2 variants is depicted in FIG. 4. Briefly, the DNA sequences for all B-GEn.2 variants mentioned in Section 8.1 were synthesized and cloned into pET29-based customized expression vector (pBLR107) using Ndel and Xhol. All constructs were expressed in BL21(DE3) E. coli cells by chemically transforming the E. coli with the coding plasmids and plating the transformed cells on antibiotic-selection LB plates. Successful colonies were scraped and transferred to 250 mL Magic Media™E. coli expression medium (Thermo Scientific) for an initial absorbance at 600 nm of 0.01. Cultures were grown first at 37° C. for 4 hours, and then at 16° C. for 40 hours. Cells were harvested and pelleted by spinning down at 5000×g for 15 minutes at 4° C. and resuspended in lysis buffer (25 mM HEPES; 500 mM NaCl, 5% glycerol, 0.5 mM TCEP and complete protease inhibitor cocktail from Sigma). Next, resuspended cells were lysed via passage through microfluidizer LM-10 (Microfluidics) 3-4 times. Cell lysates were cleared by centrifugation at 50,000×g for 30 minutes at 4° C.

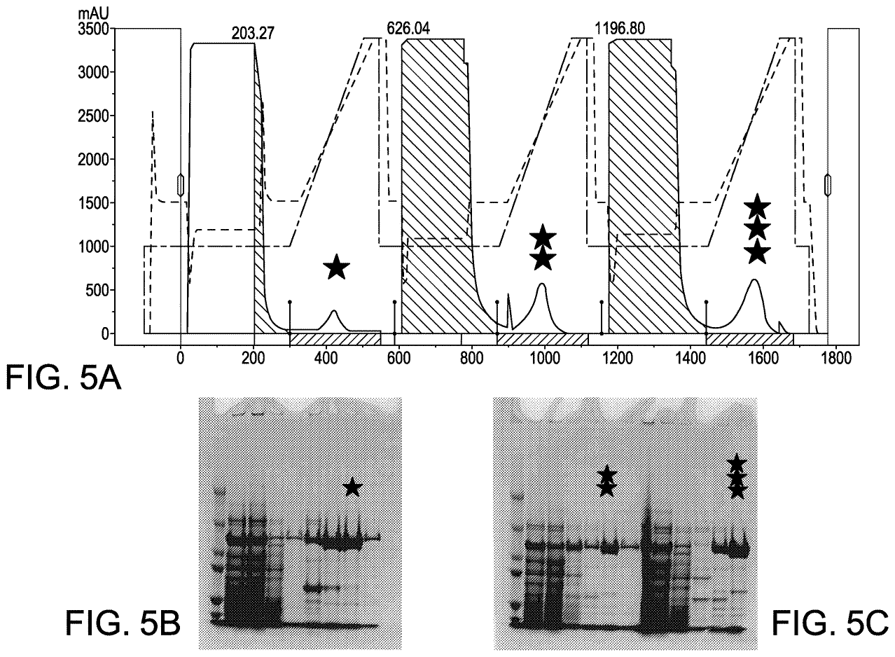

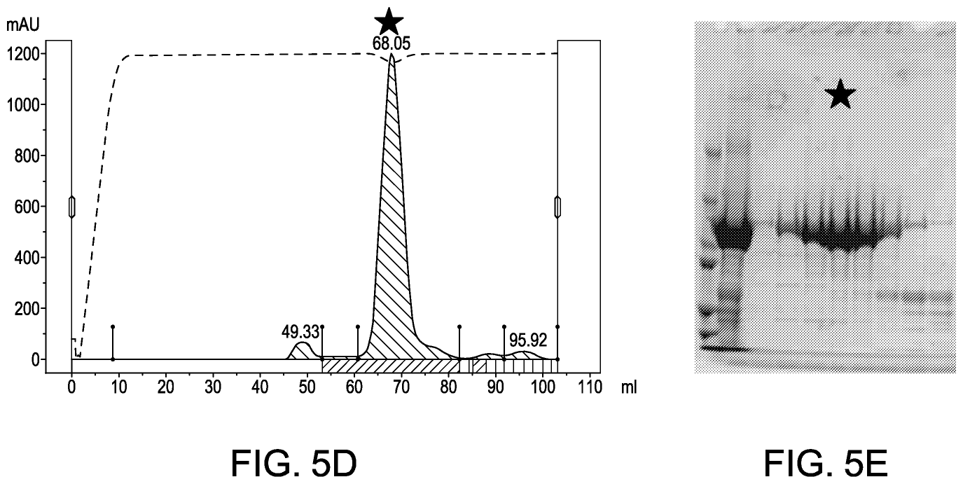

All proteins were purified using a two-step purification protocol of Heparin Fast Flow chromatography, followed by size exclusion chromatography (FIG. 5). In the first step, cleared lysates were loaded on Heparin FF columns (Cytiva) hooked onto an Akta Avant FPLC system (Cytiva) and proteins were eluted using a salt gradient. FIG. 5A shows the results of a Heparin FF chromatography, with each peak corresponding to an individual B-GEn.2 variant construct marked. The peak marked with a single star corresponds to a B-GEn.2 construct without an NLS (B-GEn.2 Full-del); the peak marked with a double star corresponds to B-GEn.2 Ndel; and the peak marked with a triple star corresponds to B-GEn.2-C-del (FIG. 5A). SDS-PAGE images confirmed that the protein fractions corresponding to the marked peaks indeed contained mostly the B-GEn.2 variants of interest with relatively low levels of contamination (FIGS. 5B and 5C).

For the second step of the purification protocol, the protein fractions marked with stars in FIG. 5A-C were pooled and injected onto a Superdex 200 size exclusion column (Cytiva) for size-based separation step. Again, the protein under the monomeric peak was pooled and concentrated to −20 mg/mL, and protein aliquots were kept frozen at −80° C. until further use. FIG. 5D shows a size exclusion chromatography purification result of B-GEn.2 Full-del, with the monomeric peak marked with a single star. SDS-PAGE of protein fractions obtained from the size exclusion purification step indicate further concentration and purification of the construct (FIG. 5E).

### 8.4. Example 4: In Cellula Editing of B2M Locus in iPSCs

### 8.4.1. Gene Editing Workflow

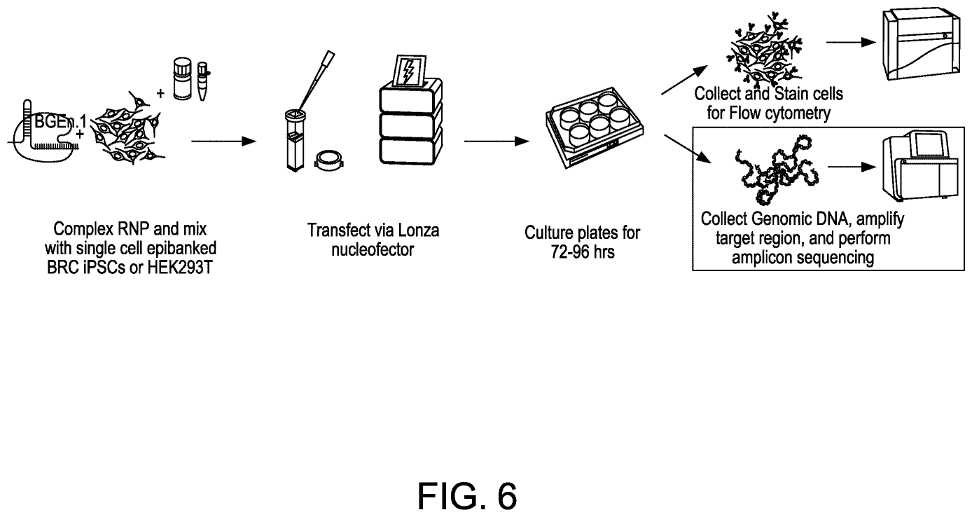

The general workflow for gene editing using B-GEn.2 variants is depicted in FIG. 6. Briefly, iPSCs were cultured in substrate-coated T75 flasks with Essential 8 (E8) growth medium and maintained at 37° C. at 5% CO₂ level between passages. Passages were done at 75 to 80% confluency. On the day of nucleofection iPSCs were detached from the flask using Accutase™ cell detachment solution (Stem Cell Technologies) by incubating for 10 minutes at 37° C. and then quenching with equal volume of E8 medium. Cell pellet was harvested by centrifugation at 115×g for 3 minutes, followed by resuspension in Lonza P3 primary cell nucleofection buffer. Ribonucleoproteins (RNPs) were assembled with sgRNAs for each nuclease construct using 1:2 ratio of protein: sgRNA (IDT). Nucleofection of complexed RNP into P3-resuspended iPSCs was accomplished using LONZA 4D nucleofector. The nucleofected cells were then plated at 150,000 cells per well in substrate-coated 24-well Falcon flat bottom plates (Corning) and grown in E8 growth medium (with Rock inhibitor Y-27632 (Tocris) for 72 to 96 hours. After harvesting, some of the cells were stained for flow cytometry using APC-conjugated antibody against B2M (for B2M-targeted experiments only) from BioLegend. The remaining cells were resuspended in 30 pL lysis buffer from BioRad's singleshot cell lysis kit and crude gDNA extraction done by incubation at room temperature for 10 minutes, then at 37° C. for 5 minutes, followed by proteinase K inactivation at 75° C. for 5 minutes. 1 μL of crude gDNA extract was directly used for every 25 μL of PCR reaction for amplicon sequencing first step, followed by end-prep and indexing and sequencing on an Illumina MiSeq sequencer.

### 8.4.2. Results

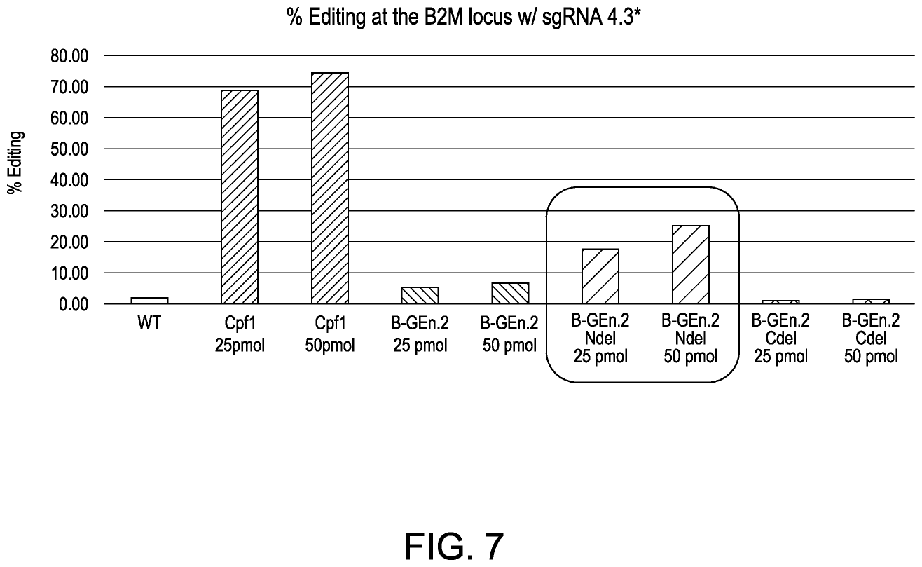

The RNPs comprising v4.3* sgRNA and B-GEn.2 construct flanked by NLS at both N- and C-termini (1151) displayed low levels of gene editing, with an editing percentage of 5.4 when 25 pmol of RNP was used and 6.83 when 50 pmol of RNP was used (FIG. 7). This low level of gene editing was likely not due to suboptimal conditions or experimental inefficiencies as indicated by relatively high levels of gene editing by RNP made of Cpf1/Cas12a and its B2M targeting sgRNA, which displayed editing percentage of 68.70 when 25 pmol of RNP was used and 74.48 when 50 pmol of RNP was used (FIG. 7). Deletion of the C-terminus NLS further reduced the editing efficiency of B-GEn.2 Cdel construct to a percent editing level (editing percentages of 0.93 and 1.41, with 25 and 50 pmol of RNP, respectively) indistinguishable from the control with no Cas nuclease (an editing percentage of 1.75). This reduction might be due to failure of nuclear localization of B-GEn.2 Cdel by its N-terminal Nucleoplasmin NLS. The editing percentage of RNPs with B-GEn.2 Ndel was 17.74 at 25 pmol and 25.09 at 50 pmol. Hence, the deletion of the N-terminal NLS improved gene editing approximately 3.5-fold, relative to the editing achieved by B-GEn.2 flanked by NLS on both sides (FIG. 7).

### 8.5. Example 5: In Cellula Editing of Albumin Locus in iPSCs

Using the same gene editing workflow described in Section 8.4.1, B-GEn.2 constructs were evaluated for editing of albumin locus in iPSCs. This time RNPs were assembled with either v4.3* or v4.4* sgRNAs for each nuclease construct, using 1:2 ratio of protein:sgRNA.

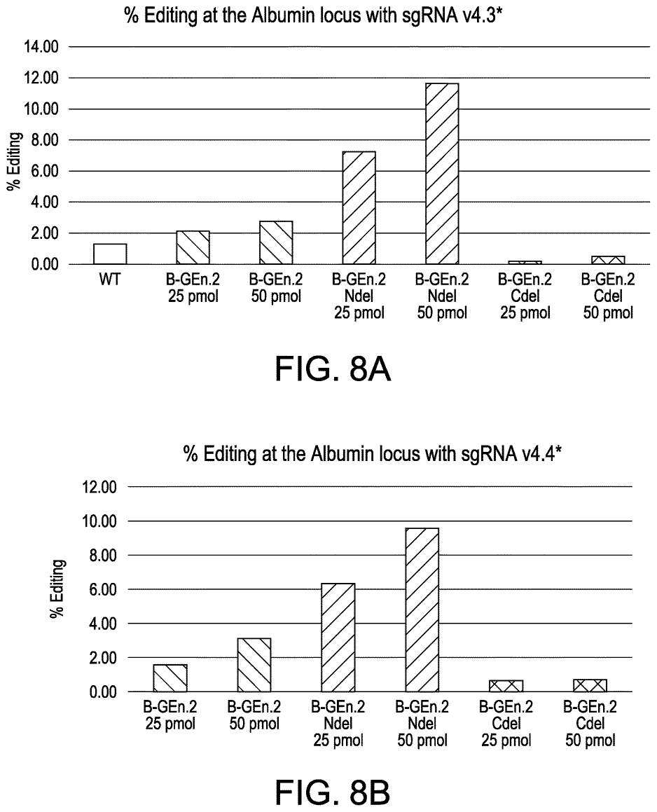

RNPs comprising v4.3* sgRNA and B-GEn.2 with NLS at both termini displayed low levels of gene editing, with an editing percentage of 2.16 for 25 pmol of RNP and 2.79 for 50 pmol of RNP (FIG. 8A). The deletion of the C-terminus NLS further reduced the editing efficiency of B-GEn.2 Cdel construct to a percent editing level (editing percentages of 0.17 and 0.51, with 25 and 50 pmol of RNP, respectively) below what was obtained with a negative control (an editing percentage of 1.29). However, the deletion of the N-terminal NLS enhanced gene editing. This time, the editing percentage of RNPs with B-GEn.2 Ndel was 7.30 at 25 pmol and 11.45 at 50 pmol (FIG. 8A). Consistent with the observations in Section 8.4.2, the deletion of the N-terminal NLS improved gene editing at the albumin locus between 3.3 and 4.1-fold, relative to editing by B-GEn.2 flanked by NLS on both sides (FIG. 8A). Similar results were achieved when v4.4* sgRNA was used instead of v4.3*. In this case, B-GEn.2 with NLS at both termini displayed an editing percentage of 1.58 for 25 pmol of RNP and 3.14 for 50 pmol of RNP, and the deletion of the C-terminus NLS further reduced the editing efficiency of B-GEn.2 Cdel construct to an editing percentage of 0.62 for 25 pmol and 0.68 for 50 pmol (FIG. 8B). The N-terminal deletion of NLS improved gene editing by a range of 3 and 4-fold, leading to an editing percentage of 6.34 at 25 pmol and 9.56 at 50 pmol (FIG. 8B). 8.6. Example 6: In Cellula Editing of Albumin Locus in HEK293-T Cells

A gene editing workflow similar to the protocol described in Section 8.4.1 was used with the following differences: HEK 293-T cells (ATCC CRL-3216™ 293T) were cultured in DMEM (ATCC) growth medium with 10% FBS and Pen/Strep supplementation and plates/flasks were not coated with substrate (unlike iPSCs). Nucleofection of complexed RNP in HEK293T was performed similarly as Section 8.4.1

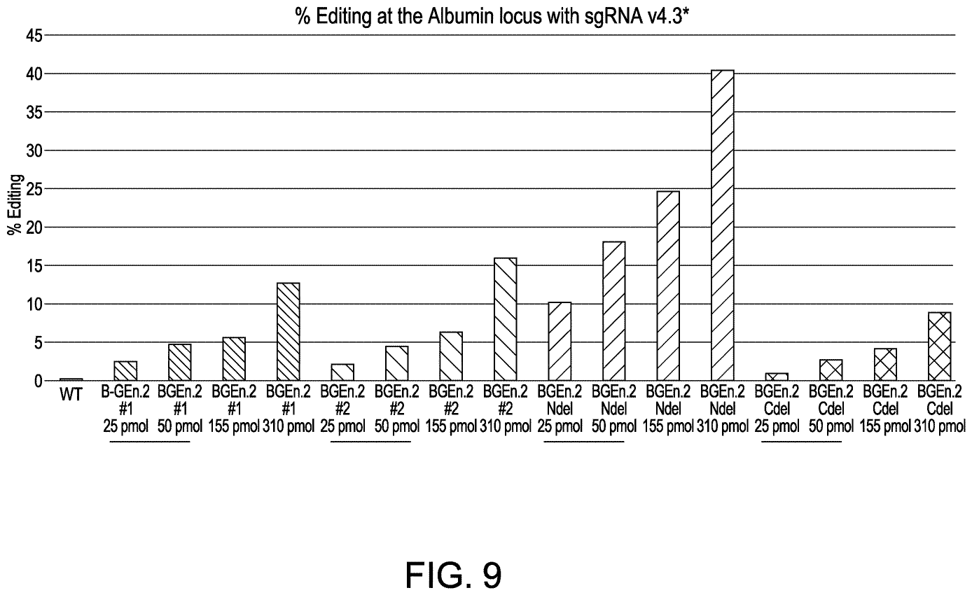

Using 25 to 310 pmol of RNPs comprising v4.3* sgRNA, B-GEn.2 variant constructs were evaluated for gene editing. The negative control was associated with a gene editing percentage of 0.18. At every RNP concentration assessed, B-GEn.2 (#1) flanked by NLS on both ends that was used in the previous examples performed similarly to the B-GEn.2 (#2) which had the same NLS configuration (B-GEn.2 (#1) gene editing percentages: 2.53 for 25 μmol, 4.8 for 50 μmol, 5.67 for 155 μmol, and 12.71 for 310 pmol; B-GEn.2 (#2) gene editing percentages: 2.1 for 25 μmol, 4.54 for 50 μmol, 6.26 for 155 μmol, and 15.99 for 310 μmol) (FIG. 9). The deletion of the C-terminus NLS further reduced the editing efficiency of B-GEn.2 Cdel construct (0.96 for 25 μmol, 2.76 for 50 μmol, 4.24 for 155 μmol, and 8.92 for 310 μmol) relative to the editing achieved by B-GEn.2 flanked by NLS on both ends. Nevertheless, the deletion of the N-terminus resulted in approximately 3.5 times higher editing percentage for all tested concentrations (10.23 for 25 μmol, 18.16 for 50 μmol, 24.72 for 155 μmol, and 40.52 for 310 μmol), when compared to the editing achieved by B-GEn.2 flanked by NLS on both ends (FIG. 9).

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

1. A fusion polypeptide comprising:
(a) a nuclease sequence, which is an amino acid sequence having at least 80% sequence identity to SEQ ID NO:6 (B-GEn.2), SEQ ID NO:5 (B-GEn.1.2), or SEQ ID NO:4 (B-GEn.1); and
(b) a first nuclear localization signal (“NLS”) sequence C-terminal to the nuclease sequence,
wherein the fusion polypeptide lacks a NLS sequence N-terminal to the nuclease sequence.

2. The fusion polypeptide of claim 1, which further comprises a first linker sequence between the nuclease sequence and the first NLS sequence.

3. The fusion polypeptide of claim 1, which further comprises a second NLS sequence C-terminal to the first NLS sequence, optionally comprising a linker sequence between the first NLS sequence and the second NLS sequence.

4. The fusion polypeptide of claim 3, which further comprises a third NLS sequence C-terminal to the second NLS sequence, optionally comprising a linker sequence between the second NLS sequence and the third NLS sequence.

5. The fusion polypeptide of claim 1, wherein the nuclease sequence comprises an amino acid sequence that differs from the amino acid sequence of SEQ ID NO:6 (B-GEn.2) by no more than 25 amino acids.

6. The fusion polypeptide of claim 1, wherein the nuclease sequence comprises an amino acid sequence that differs from the amino acid sequence of SEQ ID NO:5 (B-GEn.1.2) by no more than 25 amino acids.

7. The fusion polypeptide of claim 1, wherein the nuclease sequence comprises an amino acid sequence that differs from the amino acid sequence of SEQ ID NO:4 (B-GEn.1) by no more than 25 amino acids.

8. The fusion polypeptide of claim 1, which comprises or consists of the amino acid sequence of SEQ ID NO:2.

9. A nucleic acid comprising a nucleotide sequence encoding the fusion polypeptide of claim 1.

10. The nucleic acid of claim 9, wherein the nucleotide sequence encoding the fusion polypeptide of claim 1 is operably linked to a promoter.

11. The nucleic acid of claim 9, wherein the nucleic acid further encodes a guide RNA.

12. The nucleic acid of claim 10, which is in the form of a vector.

13. The nucleic acid of claim 12, wherein the vector is an expression vector.

14. The nucleic acid of claim 13, wherein the expression vector is a production vector.

15. The nucleic acid of claim 13, wherein the expression vector is a delivery vector.

16. The nucleic acid of claim 12, wherein the vector is an RNA vector.

17. The nucleic acid of claim 12, wherein the vector is a DNA vector.

18. A cell engineered to express a nucleotide sequence encoding the fusion polypeptide of or comprising the nucleic acid of claim 9.

19. The cell of claim 18, which is a stem cell.

20. The cell of claim 18, wherein the stem cell is a human stem cell, optionally wherein the human stem cell is an induced pluripotent stem cell (iPSC).

21. A method of producing a fusion polypeptide according to claim 1, comprising culturing the cell of claim 18 under conditions in which the fusion polypeptide is produced.

22. The method of claim 21, which further comprises isolating and/or purifying the fusion polypeptide.

23. A composition comprising:
(a) a fusion polypeptide according to claim 1; and
(b) a guide RNA.

24. The composition of claim 23, which is a ribonucleoprotein complex.

25. A method of editing the genome of a cell, comprising introducing into the cell:
(a) (i) the fusion polypeptide of claim 1; and
(ii) a guide RNA;
(b) one or more nucleic acids, optionally in the form of a vector according to claim 15, encoding:
(i) the fusion polypeptide of claim 1; and
(ii) a guide RNA,
(c) the composition of claim 23; or
(d) any combination of two or all three of (a)-(c).

26. The method of claim 25, wherein the cell is a stem cell.

27. The method of claim 25, wherein the cell is a human stem cell, optionally wherein the human stem cell is an induced pluripotent stem cell (iPSC).

28. A stem cell comprising:
(a) the composition of claim 23; or
(b) a nucleic acid according to claim 9.

29. The stem cell of claim 28, which is a human stem cell, optionally wherein the human stem cell is an induced pluripotent stem cell (iPSC).
